(19) United States
(12) Patent Application Publication
(10) Pub. No.: US 2022/0389090 A1
(43) Pub. Date: Dec. 8, 2022

(54) USE OF IL-1BETA BINDING ANTIBODIES

(71) Applicant: NOVARTIS AG, Basel (CH)

(72) Inventors: Margaret Han DUGAN, Woodside, NY (US); Yi Yang LAU, Chatham, NJ (US); Peter LIBBY, Boston, MA (US); Monica LIGUEROS-SAYLAN, Basking Ridge, NJ (US); Patrice Tinaye MATCHABA, Califon, NJ (US); Penelope OTTEWELL, Sheffield (GB); Paul RIDKER, Chestnut Hill, MA (US); Tom THUREN, Succasunna, NJ (US)

(21) Appl. No.: 17/572,228

(22) Filed: Jan. 10, 2022

Related U.S. Application Data
(63) Continuation of application No. 15/970,542, filed on May 3, 2018, now abandoned.
(60) Provisional application No. 62/649,631, filed on Mar. 29, 2018.
(60) Provisional application No. 62/596,054, filed on Dec. 7, 2017.
(60) Provisional application No. 62/550,325, filed on Aug. 25, 2017.
(60) Provisional application No. 62/550,307, filed on Aug. 25, 2017.
(60) Provisional application No. 62/529,515, filed on Jul. 7, 2017.
(60) Provisional application No. 62/523,458, filed on Jun. 22, 2017.

(51) Int. Cl. C07K 16/24; A61K 39/00; A61P 35/00; A61K 9/00; G01N 33/574

(52) U.S. Cl. CPC C07K 16/245; A61K 39/00114; A61P 35/00; A61K 9/0019; G01N 33/57423; A61K 2039/505; A61K 2039/836; A61K 2039/54; A61K 2039/828; A61K 2039/868; A61K 2039/545; A61K 2039/86; A61K 2039/82; A61K 31/282

(57) ABSTRACT

Use of an IL-1β binding antibody or a functional fragment thereof, especially canakinumab or a functional fragment thereof, or gevokizumab or a functional fragment thereof, and biomarkers for the treatment and/or prevention of cancer with at least partial inflammatory basis.

### CROSS REFERENCE TO RELATED APPLICATIONS

This application claims the benefit of U.S. Provisional Application No. 62/649,631, filed on Mar. 29, 2018; U.S. Provisional Application No. 62/596,054, filed on Dec. 7, 2017; U.S. Provisional Application No. 62/550,307, filed on Aug. 25, 2017; U.S. Provisional Application No. 62/550,325, filed on Aug. 25, 2017; U.S. Provisional Application No. 62/529,515, filed on Jul. 7, 2017; and U.S. Provisional Application No. 62/523,458, filed on Jun. 22, 2017, each of which is hereby incorporated by reference in its entirety.

### SEQUENCE LISTING

The instant application contains a Sequence Listing, which was submitted electronically in ASCII format via EFS-Web on Oct. 30, 2018, and is hereby incorporated by reference in its entirety.

### TECHNICAL FIELD

The present invention relates to the use of an IL-1β binding antibody or a functional fragment thereof for the treatment and/or prevention of cancer having at least a partial inflammatory basis, including lung cancer.

### BACKGROUND OF THE DISCLOSURE

Lung cancer is one of the most common cancers worldwide among both men and women. Lung cancer is classified into two types: small cell lung cancer (SCLC) and non-small cell lung cancer (NSCLC). The types are distinguished on the basis of histological and cytological observations, with NSCLC accounting for approximately 85% of lung cancer cases. Non-small cell lung cancer is further classified into subtypes, including but not limited to, squamous cell carcinoma, adenocarcinoma, bronchioalveolar carcinoma, and large cell (undifferentiated) carcinoma. Despite a variety of treatment option, the 5-year survival rates are only between 10% and 17%. Thus, there remains a continued need to develop new treatment options for lung cancer.

Similarly, although the current standard of care has provided significant outcome improvement for other cancers having at least a partial inflammatory basis, the vast majority of patients have incurable disease with limited survival for patients who progressed on chemotherapy.

### SUMMARY OF THE DISCLOSURE

The present disclosure relates to the use of an IL-1β binding antibody or a functional fragment thereof, for the treatment and/or prevention of cancers that have at least a partial inflammatory basis, especially lung cancer. Typically other cancers that have at least a partial inflammatory basis include colorectal cancer (CRC), melanoma, gastric cancer (including esophageal cancer), renal cell carcinoma (RCC), breast cancer, prostate cancer, head and neck cancer, bladder cancer, hepatocellular carcinoma (HCC), ovarian cancer, cervical cancer, endometrial cancer, pancreatic cancer, neuroendocrine cancer, multiple myeloma, acute myeloblastic leukemia (AML), and biliary tract cancer.

An object of the present invention is to provide a therapy to improve the treatment of cancer having at least a partial inflammatory basis, including lung cancer. The present invention therefore relates to a novel use of an IL-1β binding antibody or a functional fragments thereof, suitably canakinumab, suitably gevokizumab, for the treatment and/or prevention of cancer having at least a partial inflammatory basis, including lung cancer. In another aspect, the present invention relates to a particular clinical dosage regimen for the administration of an IL-1β binding antibody or a functional fragment thereof for the treatment and/or prevention of cancer having at least a partial inflammatory basis, including lung cancer. In another aspect the subject with cancer having at least a partial inflammatory basis, including lung cancer, is administered with one or more chemotherapeutic agent and/or have received/will receive debulking procedures in addition to the administration of an IL-1β binding antibody or a functional fragment thereof.

There are also provided methods of treating or preventing cancer having at least a partial inflammatory basis, including lung cancer, in a human subject in need thereof comprising administering to the subject a therapeutically effective amount of an IL-1β binding antibody or a functional fragment thereof.

Another aspect of the invention is the use of an IL-1β binding antibody or a functional fragment thereof for the preparation of a medicament for the treatment of cancer having at least a partial inflammatory basis, including lung cancer.

The present disclosure also provides a pharmaceutical composition comprising a therapeutically effective amount of an IL-1β binding antibody or a functional fragment thereof, suitably canakinumab, for use in the treatment and/or prevention of cancer having at least a partial inflammatory basis, including lung cancer, in a patient.

The present invention also relates to high sensitivity C-reactive protein (hsCRP) for use as a biomarker in the treatment and/or prevention of cancer having at least a partial inflammatory basis, including lung cancer, in a patient. In a further aspect the invention relates to high sensitivity C-reactive protein (hsCRP) for use as a biomarker in the treatment and/or prevention of cancer having at least a partial inflammatory basis, including lung cancer, in a patient, wherein said patient is treated with an IL-1β inhibitor, an IL-1β binding antibody or a functional fragment thereof.

In one aspect the present invention provides an IL-1β binding antibody or a functional fragment thereof for use in a male patient in need thereof in the treatment and/or prevention of a cancer having at least partial inflammatory basis, including lung cancer.

In one aspect the present invention provides an IL-1β binding antibody or a functional fragment thereof for use in a patient in need thereof in the treatment and/or prevention of a cancer having at least partial inflammatory basis, excluding lung cancer. Each and every embodiments disclosed in this application applies, separately or in combination, to this aspect.

In one aspect the present invention provides an IL-1β binding antibody or a functional fragment thereof for use in a patient in need thereof in the treatment and/or prevention of a cancer having at least partial inflammatory basis, excluding breast cancer. Each and every embodiments disclosed in this application applies, separately or in combination, to this aspect.

In one aspect the present invention provides an IL-1β binding antibody or a functional fragment thereof for use in a patient in need thereof in the treatment and/or prevention of a cancer having at least partial inflammatory basis, excluding lung cancer and corrolectal cancer. Each and every embodiments disclosed in this application applies, separately or in combination, to this aspect.

### FIGURE LEGENDS

FIG. 1. CANTOS trial profile.

FIGS. 2-4. Cumulative incidence of fatal cancer (FIG. 2), lung cancer (FIG. 3), and fatal lung cancer (FIG. 4) among CANTOS participants randomly allocated to placebo, canakinumab 50 mg, canakinumab 150 mg, or canakinumab 300 mg.

FIG. 5. Forest plot for hazard ratio (confirmed lung cancer patients)−300 mg vs placebo.

FIG. 6. Median change from baseline in hsCRP at month 3 by treatment arm (confirmed Lung cancer analysis set).

FIG. 7. In vivo model of spontaneous human breast cancer metastasis to human bone predicts a key role for IL-1β signaling in breast cancer bone metastasis. Two 0.5 cm³ pieces of human femoral bone were implanted subcutaneously into 8-week old female NOD SCID mice (n=10/group). 4 weeks later luciferase labelled MDA-MB-231-luc2-TdTomato or T47D cells were injected into the hind mammary fat pads. Each experiment was carried out 3-separate times using bone form a different patient for each repeat. Histograms showing fold change of IL-1B, IL-1R1, Caspase 1 and IL-1Ra copy number (dCT) compared with GAPDH in tumour cells grown in vivo compared with those grown in a tissue culture flask (a i); mammary tumours that metastasise compared with mammary tumours tumours that do not metastasise (a ii); circulating tumour cells compared with tumour cells that remain in the fat pad (a iii) and bone metastases compared with the matched primary tumour (a iv). Fold change in IL-1β protein expression is shown in (b) and fold change in copy number of genes associated with EMT (E-cadherin, N-cadherin and JUP) compared with GAPDH are shown in (c). *=P<0.01**=P<0.001, ***=P<0.0001, \hat{ }\hat{ }\hat{ }=P<0.001 compared with naïve bone.

FIG. 8. Stable transfection of breast cancer cells with IL-1B. MDA-MB-231, MCF7 and T47D breast cancer cells were stably transfected with IL-1B using a human cDNA ORF plasmid with a C-terminal GFP tag or control plasmid. a) shows pg/ng IL-1β protein from IL-1β-positive tumour cell lysates compared with scramble sequence control. b) shows pg/ml of secreted IL-1β from 10,000 IL-1β+ and control cells as measured by ELISA. Effects of IL-1B overexpression on proliferation of MDA-MB-231 and MCF7 cells are shown in (c and d) respectively. Data shown are mean+/−SEM, *=P<0.01, **=P<0.001, ***=P<0.0001 compared with scramble sequence control.

FIG. 9. Tumour derived IL-1β induces epithelial to mesenchymal transition in vitro. MDA-MB-231, MCF7 and T47D cells were stably transfected with to express high levels of IL-1B, or scramble sequence (control) to assess effects of endogenous IL-1B on parameters associated with metastasis. Increased endogenous IL-1B resulted tumour cells changing from an epithelial to mesenchymal phenotype (a). b) shows fold-change in copy number and protein expression of IL-1B, IL-1R1, E-cadherin, N-cadherin and JUP compared with GAPDH and β-catenin respectively. Ability of tumour cells to invade towards osteoblasts through Matrigel and/or 8 μM pores, are shown in (c) and capacity of cells to migrate over 24 and 48 h is shown using a wound closure assay (d). Data are shown as mean+/−SEM, *=P<0.01, **=P<0.001, ***=P<0.0001.

FIG. 10. Pharmacological blockade of IL-1B inhibits spontaneous metastasis to human bone in vivo. Female NOD-SCID mice bearing two 0.5 cm³ pieces of human femoral bone received intra-mammary injections of MDA-MB-231Luc2-TdTomato cells. One week after tumour cell injection mice were treated with 1 mg/kg/day IL-1Ra, 20 mg/kg/14-days canakinumab, or placebo (control) (n=10/group). All animals were culled 35 days following tumour cell injection. Effects on bone metastases (a) were assessed in vivo and immediately post-mortem by luciferase imaging and confirmed ex vivo on histological sections. Data are shown as numbers of photons per second emitted 2 minutes following sub-cutaneous injection of D-luciferin. Effects on numbers of tumour cells detected in the circulation are shown in (b). *=P<0.01, **=P<0.001, ***=P<0.0001.

FIG. 11. Tumour derived IL-1B promotes breast cancer bone homing in vivo. 8-week old female BALB/c nude mice were injected with control (scramble sequence) or IL-1B overexpressing MDA-MB-231-IL-1B+ cells via the lateral tail vein. Tumour growth in bone and lung were measured in vivo by GFP imaging and findings confirmed ex vivo on histological sections. a) shows tumour growth in bone; b) shows representative μCT images of tumour bearing tibiae and the graph shows bone volume (BV)/tissue volume (TV) ratio indicating effects on tumour induced bone destruction; c) shows numbers and size of tumours detected in lungs from each of the cell lines. *=P<0.01, **=P<0.001, ***=P<0.0001. (B=bone, T=tumour, L=lung)

FIG. 12. Tumour cell-bone cell interactions stimulate IL-1B production cell proliferation. MDA-MB-231 or T47D human breast cancer cell lines were cultured alone or in combination with live human bone, HS5 bone marrow cells or OB1 primary osteoblasts. a) shows the effects of culturing MDA-MB-231 or T47D cells in live human bone discs on IL-1β concentrations secreted into the media. The effect of co-culturing MDA-MB-231 or T47D cells with HS5 bone cells on IL-1β derived from the individual cell types following cell sorting and the proliferation of these cells are shown in b) and c). Effects of co-culturing MDA-MB-231 or T47D cells with OB1 (osteoblast) cells on proliferation are shown in d). Data are shown as mean+/−SEM, *=P<0.01, **=P<0.001, ***=P<0.0001.

FIG. 13. IL-1β in the bone microenvironment stimulates expansion of the bone metastatic niche. Effects of adding 40 pg/ml or 5 ng/ml recombinant IL-1β to MDA-MB-231 or T47D breast cancer cells is shown in (a) and effects on adding 20 pg/ml, 40 pg/ml or 5 ng/ml IL-1B on proliferation of HS5, bone marrow, or OB1, osteoblasts, are shown in b) and c) respectively. (d) IL-1 driven alterations to the bone vasculature was measured following CD34 staining in the trabecular region of the tibiae from 10-12-week old female IL-1R1 knockout mice. (e) BALB/c nude mice treated with 1 mg/ml/day IL-1Ra for 31 days and (f) C57BL/6 mice treated with 10 μM canakinumab for 4-96 h. Data are shown as mean+/−SEM, *=P<0.01, **=P<0.001, ***=P<0.0001.

FIG. 14. Suppression of IL-1 signalling affects bone integrity and vasculature. Tibiae and serum from mice that do not express IL-1R1 (IL-1R1 KO), BALB/c nude mice treated daily with 1 mg/kg per day of IL-1R antagonist for 21 and 31 days and C57BL/6 mice treated with 10 mg/kg of canakinumab (Ilaris) of 0-96 h were analysed for bone integrity by μCT and vasculature using ELISA for Endothelin 1 and pan VEGF. a) shows the effects of IL-1R1 KO; b) effects of Anakinra and c) effects of canakinumab on bone volume compared with tissue volume (i), concentration of Endothelin 1 (ii) and concentrations of VEGF secreted into the serum. Data shown are mean+/−SEM, *=P<0.01, **=P<0.001, ***=P<0.0001 compared with control.

FIG. 15. Tumour derived IL-1β predicts future recurrence and bone relapse in patients with stage II and III breast cancer. 1300 primary breast cancer samples from patients with stage II and III breast cancer with no evidence of metastasis were stained for 17 kD active IL-1β. Tumours were scored for IL-1β in the tumour cell population. Data shown are Kaplan Meyer curves representing the correlation between tumour derived IL-1β and subsequent recurrence a) at any site or b) in bone over a 10-year time period.

FIG. 16. Simulation of canakinumab PK profile and hsCRP profile. a) shows canakinumab concentration time profiles. Solid line and band: median of individual simulated concentrations with 2.5-97.5% prediction interval (300 mg Q12W (bottom line), 200 mg Q3W (middle line), and 300 mg Q4W (top line)). b) shows the proportion of month 3 hsCRP being below the cut point of 1.8 mg/L for three different populations: all CANTOS patients (scenario 1), confirmed lung cancer patients (scenario 2), and advanced lung cancer patients (scenario 3) and three different dose regimens. c) is similar to b) with the cut point being 2 mg/L. d) shows the median hsCRP concentration over time for three different doses. e) shows the percent reduction from baseline hsCRP after a single dose.

FIG. 17. Gene expression analysis by RNA sequencing in colorectal cancer patients receiving PDR001 in combination with canakinumab, PDR001 in combination with everolimus and PDR001 in combination with others. In the heatmap figure, each row represents the RNA levels for the labelled gene. Patient samples are delineated by the vertical lines, with the screening (pre-treatment) sample in the left column, and the cycle 3 (on-treatment) sample in the right column. The RNA levels are row-standardized for each gene, with black denoting samples with higher RNA levels and white denoting samples with lower RNA levels. Neutrophil-specific genes FCGR3B, CXCR2, FFAR2, OSM, and GOS2 are boxed.

FIG. 18. Clinical data after gevokizumab treatment (panel a) and its extrapolation to higher doses (panels b, c, and d). Adjusted percent change from baseline in hsCRP in patients in a). The hsCRP exposure-response relationship is shown in b) for six different hsCRP base line concentrations. The simulation of two different doses of gevokizumab is shown in b) and c).

FIG. 19. Effect of anit-IL-1beta treatment in two mouse models of cancer. a), b), and c) show data from the MC38 mouse model, and d) and e) show data from the LL2 mouse model.

FIG. 20. Graphic showing design of phase III study CACZ885T2301.

### DETAILED DESCRIPTION OF THE DISCLOSURE

Many malignancies arise in areas of chronic inflammation (1) and inadequate resolution of inflammation is hypothesized to play a major role in tumor invasion, progression, and metastases (2-4). Inflammation is of particular pathophysiologic relevance for lung cancer where chronic bronchitis, triggered by asbestos, silica, smoking, and other external inhaled toxins, results in a persistent pro-inflammatory response (5,6). Inflammatory activation in the lung is mediated in part through activation of the Nod-like receptor protein 3 (NLRP3) inflammasome with consequent local production of interleukin-1β (IL-1β), a process that can lead to both chronic fibrosis and cancer (7, 8). In murine models, inflammasome activation and IL-1β production can accelerate tumor invasiveness, growth, and metastatic spread (2). For example, in IL-1β−/− mice, neither local tumors nor lung metastases develop following localized or intravenous inoculation with melanoma cell lines, data suggesting that IL-1β may be essential for the invasiveness of already existing malignancies (9). It has thus been hypothesized that inhibition of IL-1β might have an adjunctive role in the treatment of cancers that have at least a partial inflammatory basis (10-13).

The present invention arose from the analysis of the data generated from the CANTOS trial, which is a randomized, double-blind, placebo-controlled, event-driven trial. CANTOS was designed to evaluate whether the administration of quarterly subcutaneous canakinumab can prevent recurrent cardiovascular events among stable post-myocardial infarction patients with elevated hsCRP. The enrolled 10,061 patients with myocardial infarction and inflammatory atherosclerosis were free of previously diagnosed cancer and had high sensitivity C-reactive protein (hsCRP)≥2 mg/L. Three escalating canakinumab doses (50 mg, 150 mg, and 300 mg given subcutaneously every 3 months) were compared to placebo. Participants were followed for incident cancer diagnoses over a median follow-up period of 3.7 years.

Patient Population Patients were eligible for enrollment in CANTOS if they had a prior history of myocardial infarction and had blood levels of hsCRP≥2 mg/L despite use of aggressive secondary prevention strategies. As canakinumab is a systemic immunomodulatory agent, the trial was designed to exclude from enrollment those with a history of chronic or recurrent infections, prior malignancy other than basal cell skin carcinoma, suspected or known immunocompromised states, a history of or at high risk for tuberculosis or HIV-related disease, or ongoing use of systemic anti-inflammatory treatments.

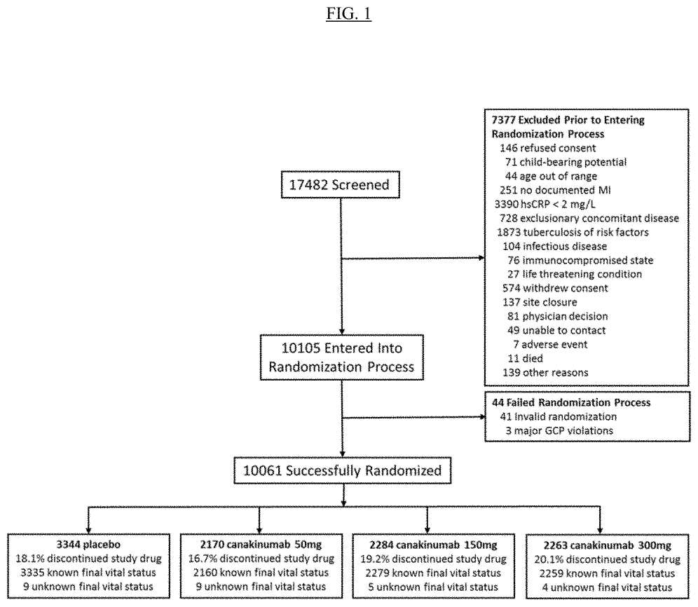

Randomization (FIG. 1) Based on experience from a phase IIb study (19), an “anchor dose” was initially selected for canakinumab of 150 mg SC every three months. In addition, a higher dose of 300 mg given twice over a two-week period and then every three months was also initially selected to address theoretical concerns regarding IL-1β auto-induction. As such, when the first patient was screened on Apr. 11, 2011, CANTOS was initiated as a three-arm trial comparing standard of care plus placebo to either standard of care plus canakinumab 150 mg or canakinumab 300 mg with participants allocated to each study arm in a 1:1:1 ratio. However, following health authority feedback requiring broader dose-response data, a lower dose canakinumab arm was introduced into the trial (50 mg SC every three months). The protocol was thus amended and a formal four arm structure was approved in July of 2011 but varied in the timing of its adoption by region and site.

To accommodate this structural change, the proportion of individuals who ultimately would be allocated to placebo was increased as was the proportion moving forward who would be randomly allocated to the 50 mg dose. Thus, the treatment allocation ratios were altered from 1:1:1 for placebo:150 mg canakinumab: 300 mg canakinumab for the first 741 participants recruited to 2:1.4:1.3:1.3 for placebo: 50 mg canakinumab: 150 mg canakinumab: 300 mg canakinumab, respectively, for the remaining 9,320 participants. Trial enrolment was completed in March 2014 and all participants followed until May 2017.

Per protocol, all CANTOS participants had complete blood counts, lipid panels, hsCRP, and measures of renal and hepatic function performed at baseline and at 3, 6, 9, 12, 24, 36, and 48 months after randomization.

Endpoint Clinical endpoints of interest for the analysis were any incident cancers diagnosed and reported during trial follow-up. For any such event, medical records were obtained and the cancer diagnosis reviewed by a panel of oncologists unaware of study drug allocation. Where possible, a primary source was noted, as were any evidence of site-specific metastases. Cancers were also classified as fatal or non-fatal by the trial endpoint committee.

Statistical Analysis Cox proportional hazard models were used to analyze the incidence of cancer overall in the canakinumab and placebo groups, as well as the incidence of fatal and non-fatal cancer, and cancer incidence on a site specific basis. For proof-of-concept purposes and consistent with analyses conducted throughout the trial for all Data and Safety Monitoring Board meetings, comparisons were made between incidence rates on placebo to incidence rates for each individual canakinumab dose, across ascending canakinumab doses (with scores 0, 1, 3, and 6 proportional to dose), and for the combined active canakinumab treatment groups.

### Results

CANTOS was shown to meet the primary endpoint, demonstrating that when used in combination with standard of care, ACZ885 reduces the risk of major adverse cardiovascular events (MACE) in patients with a prior heart attack and inflammatory atherosclerosis. MACE is a composite of cardiovascular death, non-fatal myocardial infarction and non-fatal stroke. ACZ885 has been shown to reduce cardiovascular risk in people with a prior heart attack by selectively targeting inflammation.

Patients Baseline clinical characteristics of the 10,061 CANTOS participants are provided in Table 1 for those who did or did not develop a diagnosis of cancer during trial follow-up.

Compared to those who were not diagnosed with cancer, those who developed incident lung cancers were older (P<0.001), more likely to be current smokers (P<0.001). Consistent with prior work indicating a strong inflammatory component to certain cancers, median hsCRP levels were elevated at baseline among those who were diagnosed with lung cancer during follow-up compared to those who remained free of any cancer diagnosis (6.0 versus 4.2 mg/L, P<0.001). Similar data were observed for interleukin-6 (3.2 versus 2.6 ng/L, P<0.0001).

During trial follow-up, as compared to placebo, canakinumab was associated with dose-dependent reductions in hsCRP of 27 to 40 percent (all P-values<0.0001) and with dose-dependent reductions in IL-6 of 25 to 43 percent (all P-values<0.0001). Canakinumab had no effect on LDL or HDL cholesterol.

Effects on Total Cancer Events and on Fatal Cancer Events Incidence rates for any cancer in the placebo, 50 mg, 150 mg, and 300 mg canakinumab groups were 1.84, 1.82, 1.68, and 1.72 per 100 person-years, respectively (P across canakinumab dose groups compared to placebo=0.34). By contrast, a statistically significant dose-dependent effect was observed for fatal cancers where incidence rates in the placebo, 50 mg, 150 mg, and 300 mg groups were 0.64, 0.55, 0.50, and 0.31 per 100 person-years, respectively (P across canakinumab dose groups compared to placebo=0.001) (Table 2).

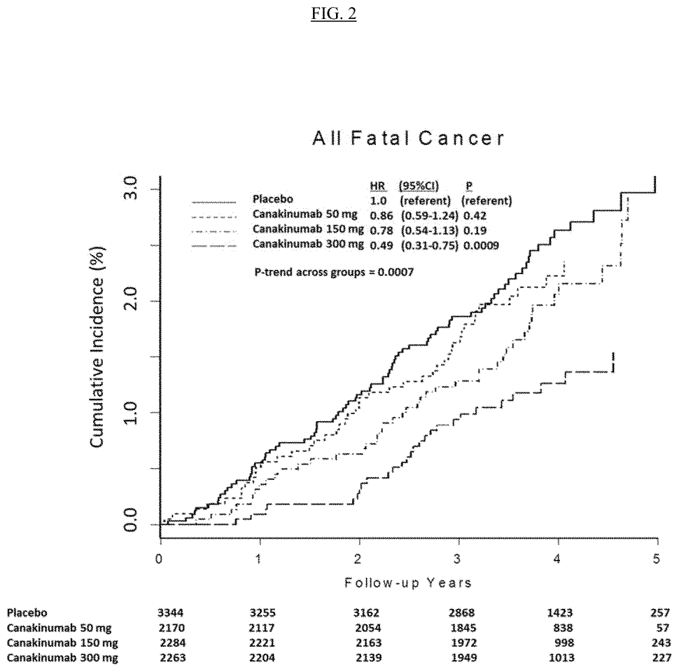

Effects on Lung Cancer Over the median 3.7 year follow-up period, random allocation to canakinumab was associated with statistically significant dose-dependent reductions in total cancer mortality. For this endpoint (N=196), referent to placebo, hazard ratios (95% confidence interval, P-value) were 0.86 (0.59-1.24, P=0.42), 0.78 (0.54-1.13, P=0.19), and 0.49 (0.31-0.75, P=0.0009) for the canakinumab 50 mg, 150 mg, and 300 mg groups, respectively. These data correspond to incidence rates in the placebo, 50 mg, 150 mg, and 300 mg groups of 0.64, 0.55, 0.50, and 0.31 per 100 person-years, respectively (P-trend across active dose groups compared to placebo=0.0007) (Table 2 and FIG. 2).

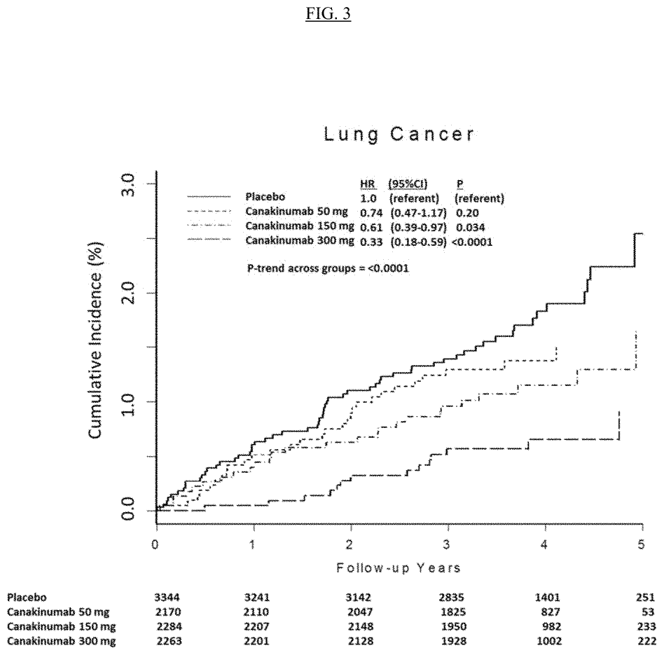

This effect was largely due to reductions in lung cancer; among those assigned to placebo, 26.0% of all cancers and 47% of all cancer deaths were lung cancers, whereas among those assigned to canakinumab, 16% of all cancers and 34% of cancer deaths were lung cancers. For incident lung cancer (N=129), referent to placebo, hazard ratios (95% confidence interval, P-value) were 0.74 (0.47-1.17, P=0.20), 0.61 (0.39-0.97, P=0.034, and 0.33 (0.18-0.59, P=0.0001) for the canakinumab 50 mg, 150 mg, and 300 mg groups, respectively. These data correspond to incidence rates in the placebo, 50 mg, 150 mg, and 300 mg groups of 0.49, 0.35, 0.30, and 0.16 per 100 person-years, respectively (P-trend across active dose groups compared to placebo<0.0001) (Table 2 and FIG. 3).

Stratification by smoking indicated slightly greater relative benefits of canakinumab on lung cancer among current as compared to past smokers (HR 0.50, P=0.005 for current smokers; HR 0.61, P=0.006 for past smokers). This effect was more prominent for the highest canakinumab dose (HR 0.25, P=0.002 for current smokers; HR 0.44, P=0.025 for past smokers, Table S2).

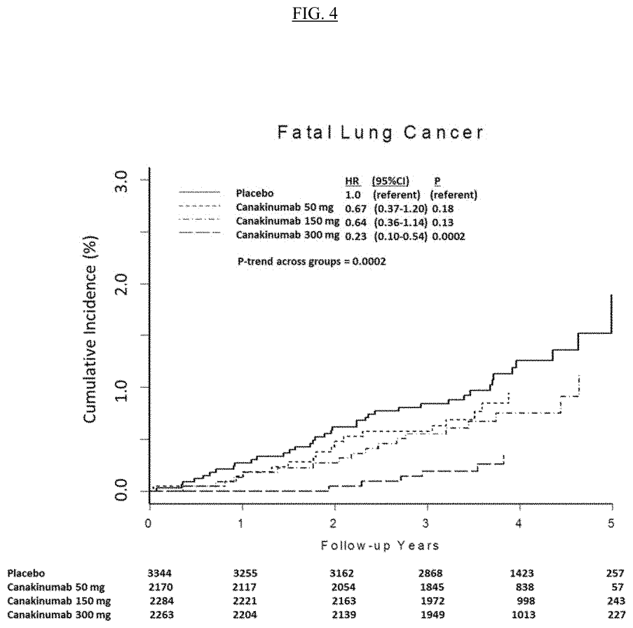

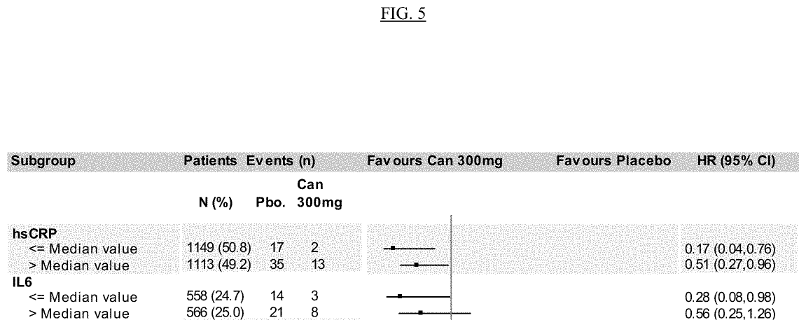

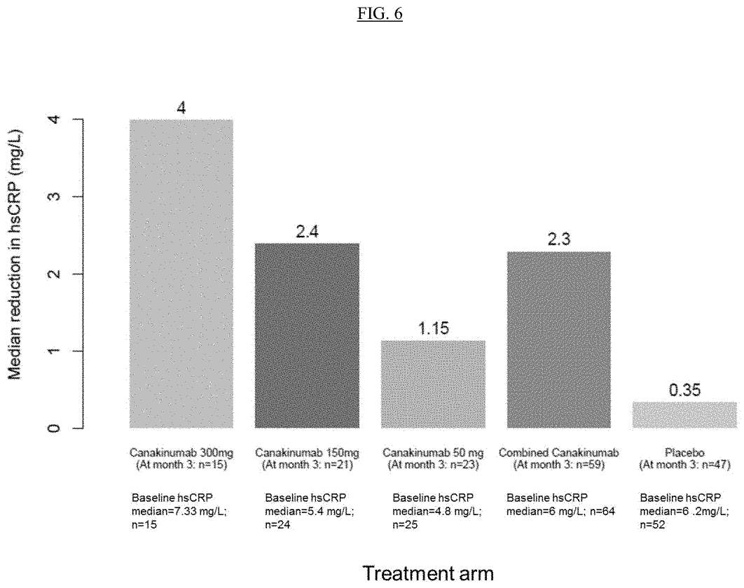

For lung cancer mortality (N=77), referent to placebo, hazard ratios (95% confidence interval, P=value) were 0.67 (0.37-1.20, P=0.18), 0.64 (0.36-1.14, P=0.13), and 0.23 (0.10-0.54, P=0.0002) for the canakinumab 50 mg, 150 mg, and 300 mg groups, respectively. These data correspond to incidence rates in the placebo, 50 mg, 150 mg, and 300 mg groups of 0.30, 0.20, 0.19, and 0.07 per 100 person-years, respectively (P-trend across active dose groups compared to placebo=0.0002) (Table 2 and FIG. 4).

Benefits of canakinumab were evident in patients for whom lung cancer type was unspecified or where histology indicated adenocarcinoma or poorly differentiated large cell cancers (incidence rates in the placebo, canakinumab 50 mg, 150 mg, and 300 mg dose groups were 0.41, 0.33, 0.27, and 0.12, respectively [P-trend across dose groups compared to placebo=0.0004]). Power was limited to definitively address effects of canakinumab in cases where histology indicated small cell lung cancers or squamous cell carcinomas (Table S3).

In analyses of combined canakinumab doses, risk reductions for total lung cancer were greater for those who had reductions in hsCRP greater than or equal to the median value at 3 months. Specifically, compared to placebo, the observed hazard ratio for lung cancer among those who achieved hsCRP reductions greater than the median value of 1.8 mg/L at 3 months was 0.29 (95% CI 0.17-0.51, P<0.0001), better than the effect observed for those who achieved hsCRP reductions less than the median value (HR 0.83, 95% CI 0.56-1.22, P=0.34). Similar effects were observed for median IL-6 levels achieved at 3 months.

While the CANTOS protocol was designed to exclude individuals with prior non-basal cell malignancies, 76 of 10,061 (0.8%) were found on detailed record review to have had prior cancers. Post-hoc exclusion of these individuals had no impact on the above results.

Adverse Events With regard to bone marrow function, thrombocytopenia and neutropenia were rare but more common among those allocated to canakinumab (Table 3). As reported elsewhere (20), while there was no increase in rates of total infections, there were increased rates of cellulitis and Clostridium difficile colitis and an increase in fatal events attributed to infection or sepsis when the three canakinumab groups were pooled and compared to placebo (incidence rates 0.31 versus 0.18 per 100 person years, P=0.023). Participants succumbing to infection tended to be older and more likely to have diabetes. Despite this adverse effect, both non-cardiovascular mortality (HR 0.97, 95% CI 0.79-1.19, P=0.80) and all-cause mortality (HR 0.94, 95% CI 0.83-1.06, P=0.31) were non-significantly reduced. Serious tuberculosis infections were rare and occurred at similar rates in the canakinumab and placebo treated groups (0.06%). Injection site reactions occurred with similar frequency in the canakinumab and placebo groups. Consistent with known effects of IL-1β inhibition, canakinumab resulted in significant reductions in adverse reports of arthritis, gout, and osteoarthritis (Table 4).

In these randomized, double-blind, placebo controlled trial data, inhibition of IL-1β with canakinumab over a median period of 3.7 years markedly reduced the rate of fatal and non-fatal lung cancer among atherosclerosis patients with elevated hsCRP who did not have a prior diagnosis of cancer. Effects were dose dependent with relative hazard reductions of 67% (P=0.0001) and 77% (P=0.0002) for total lung cancer and fatal lung cancer, respectively, among those randomly allocated to the highest canakinumab dose (300 mg SC every 3 months). Beneficial effects of canakinumab were observed on incident lung cancers within weeks of initiating therapy, again particularly at the highest canakinumab dose. Patients with elevated levels of the inflammatory biomarkers hsCRP and interleukin-6 were at highest risk for incident lung cancer and appeared to gain the most benefit, as did current smokers. By contrast, canakinumab had non-significant effects on site-specific cancers other than lung cancer. Yet for those randomly allocated to canakinumab 300 mg SC, total cancer mortality fell by half (P=0.0009).

CANTOS was an inflammation reduction trial conducted among post-myocardial infarction patients with elevated hsCRP and high rates of current or past smoking (17). These characteristics put the CANTOS population at higher than average risk for lung cancer and afforded the additional opportunity reported here to address the effect of interleukin-1β inhibition on cancer. However, by design, there are no data for individuals free of atherosclerotic disease or with low levels of hsCRP.

While possible, it is perhaps unlikely that canakinumab had any direct effects on oncogenesis and the development of new lung cancers. Patients who developed lung cancer during follow-up were 65 years of age on average on study entry and more than 90% were current or past smokers. Further, the average follow-up time is unlikely to be adequate to demonstrate a reduction in new cancers.

Rather, it seems far more likely that canakinumab—a powerful inhibitor of interleukin-1β-substantially reduced the rate of progression, invasiveness, and metastatic spread of lung cancers that were prevalent but undiagnosed at trial entry. In this regard, the clinical data are consistent with prior experimental work indicating that cytokines such as IL-1β can promote angiogenesis and tumor growth and that IL-1β is required for tumor invasiveness of already existing malignant cells (2-4,9). In murine models, high IL-1β concentrations within the tumor micro-environment are associated with more virulent phenotypes (13) and secreted IL-1β derived from this microenvironment (or directly from malignant cells) can promote tumor invasiveness and in some cased induce tumor-mediated suppression (2,9,21).

Breast cancer bone metastases is incurable and associates with poor prognosis in patients. Bone metastases occur when tumor cells are disseminated into the bone marrow and take up residence in the bone metastatic niche. This niche is thought to be made up of three interacting niches: the osteoblastic, vascular and hematopoietic stem cell niche (reviewed by (Massague and Obenauf, 2016; Weilbaecher et al., 2011)). Evidence from metastases in other organs predicts that proliferation of vascular endothelial cells and sprouting of new blood vessels may also promote proliferation of tumor cells in bone driving metastases formation (Carbonell et al., 2009; Kienast et al., 2010). It was previously shown that bone seeking breast cancer cell lines, MDA-IV produce high concentrations of IL-1β compared to parental MDA-MB-231 cells (Nutter et al., 2014). Similarly, in a PC3 model of prostate cancer genetic overexpression of IL-1β increased bone metastases from tumor cells injected into the heart whereas genetic knockdown of this molecule reduced bone metastasis (Liu et al., 2013).

Since the time of Virchow, inflammation has been linked to cancer; as Balkwill and Mantovani have written, ‘if genetic damage is “the match that lights the fire” of cancer, some types of inflammation may provide the “fuel that feeds the flames”’ (22). This hypothesis helps to explain, in part, why the chronic use of aspirin as well as other non-steroidal anti-inflammatory agents is associated with reduced fatality rates from colorectal cancer and lung adenocarcinomas (23,24). However, in contrast to these agents which require a decade or more of use to show efficacy, beneficial effects of canakinumab on lung cancer incidence and lung cancer mortality were observed in a trial with much shorter time frame. The apparent beneficial effects of canakinumab were observed within weeks of initiating therapy. The specificity of canakinumab in the data for lung cancer and its augmented effect among current smokers is of particular interest given the fact that inflammasome mediated production of IL-1β is triggered by multiple inhaled environmental toxins known to induce local pulmonary inflammation as well as cancer (7,8).

The trial was not designed as a cancer treatment study. Rather, by design, the trial enrolled atherosclerosis patients without a prior history of cancer. There is precedent for such an IL-1 targeted cytokine approach for other cancer types. For example, the IL-1 receptor antagonist anakinra has been reported in a case series of 47 patients to modestly reduce the progression of smoldering or indolent myeloma (25). In a second case series of 52 patients with diverse metastatic cancers, a human monoclonal antibody targeting IL-1α was well tolerated and showed modest improvement in lean body mass, appetite, and pain (26).

In conclusion, these randomized placebo-controlled trial data provide evidence that inhibiting innate immune function with canakinumab, a monoclonal antibody that targets IL-1β, substantially reduces incident lung cancer and lung cancer fatality.

Thus in one aspect, the present invention provides the use of an IL-1β binding antibody or a functional fragment thereof (DRUG of the invention), suitably canakinumab or a functional fragment thereof (included in DRUG of the invention), gevokizumab or a functional fragment thereof (included in DRUG of the invention), for the treatment and/or prevention of cancers that have at least a partial inflammatory basis, especially lung cancer.

In one embodiment the lung cancer has concomitant inflammation activated or mediated in part through activation of the Nod-like receptor protein 3 (NLRP3) inflammasome with consequent local production of interleukin-1β.

Advanced studies in delineating interaction between tumor and the tumor microenvironment have revealed that chronic inflammation can promote tumor development, and tumor fuels inflammation to facilitate tumor progression and metastasis. Inflammatory microenvironment with cellular and non-cellular secreted factors provides a sanctuary for tumor progression by inducing angiogenesis; recruiting tumor promoting, immune suppressive cells and inhibiting immune effector cell mediated anti-tumor immune response. One of the major inflammatory pathways supporting tumor development and progression is IL-1β, a pro-inflammatory cytokine produced by tumor and tumor associated immune suppressive cells including neutrophils and macrophages in tumor microenvironment.

The meaning of “cancers that have at least a partial inflammatory basis” or “cancer having at least a partial inflammatory basis” is well known in the art. In one embodiment, the term as used herein refers to any cancer in which the IL-1β mediated inflammatory responses contribute to the tumor development and/or propagation, including but not necessarily limited to metastasis. It is quite common that such cancer has concomitant inflammation activated or mediated in part through activation of the Nod-like receptor protein 3 (NLRP3) inflammasome with consequent local production of interleukin-1β. It is quite common that in a patient with such cancer, the expression, or even the overexpression of IL-1β can be detected, commonly at the site of the tumor, especially in the surrounding tissue of the tumor, in comparison to normal tissue. The expression of IL-1β can be detected by routine methods, such as immunostaining, ELISA based assays, ISH, RNA sequencing or RT-PCR in the tumor as well as in serum/plasma. The expression or higher expression of IL-1β can be concluded against negative control, usually normal tissue at the same site or higher than normal level of IL-1β. Simultaneously or alternatively, it is quite common that a patient with such cancer has chronic inflammation, which is manifested, typically, by higher than normal level of CRP or hsCRP, IL-6 and TNFα. Cancers that have at least a partial inflammatory basis include but not limited to lung cancer, especially NSCLC, colorectal cancer, melanoma, gastric cancer (including gastric and intestinal cancer, cancer of the esophagus, particularly the lower part of the esophagus, renal cell carcinoma (RCC), breast cancer, prostate cancer, head and neck cancer (including HPV, EBV and tobacco/alcohol included head and neck cancer), bladder cancer, hepatocellular carcinoma (HCC), pancreatic cancer, ovarian cancer, cervical cancer, endometrial cancer, neuroendocrine cancer and biliary tract cancer (including bile duct and gallbladder cancers) as well as hematologic cancers such as acute myeloblastic leukemia (AML), myelofibrosis and multiple myeloma (MM).

Available techniques allow detection and quantification of IL-1β in tissue as well as in serum/plasma, especially when the IL-1β is expressed to a higher than normal level. For example, Using the R&D Systems high sensitivity IL-1b ELISA kit, IL-1β cannot be detected in majority of healthy donor serum samples.

### Sample Values

serum/Plasma—Samples from apparantly healthy volunteers were evaluated for the presence of human IL-1β in this assay. No medical histories were available for the donors used in this study.

| | Mean of | % | Range |
|---|---|---|---|
| Sample Type | Detectable (pg/ml) | Detectable | (pg/ml) |
| Serum (n = 50) | 0.357 | 10 | ND-0.606 |
| EDTA plasma (n = 50) | 0.292 | 12 | ND-0.580 |
| Heparin plasma (n = 50) | 0.448 | 14 | ND-1.08 |

ND = Non-detectable

Thus in a healthy person the IL-1β level is bearly detectable or just above the detection limit with the high sensitivity R&D IL-1β ELISA kit. It is expected that in patients with cancer having at least partial inflammatory basis the IL-1β level will be higher than normal and can be detected by the same kit. Taking the IL-1β expression level in a healthy person as the normal level (reference level), the term “higher than normal level of IL-1β” is understood as an IL-1β level that is higher than the reference level. Normally at least 2 fold, at least 5 fold, at least 10 fold, at least 15 fold, at least 20 fold of the reference level is considered as higher than normal level. Blocking IL-1β pathway normally triggers the compensating mechanism leading to more production of IL-1β. Thus the term “higher than normal level of IL-1β” refers to the level of IL-1β either prior to or post to the administration of an IL-1β inhibitor, preferably IL-1β binding antibody or a fragment thereof. Preferably the term “higher than normal level of IL-1β” refers to the level of IL-1β prior to the administration of IL-1β inhibitor. It is also observed that treatment of cancer with agents other than IL-1β inhibitors could result in more production of IL-1β. Thus the term “higher than normal level of IL-1β” refers to the level of IL-1β either prior to or post to the administration of said agents.

When using staining, such as immunstaining, to detect IL-1β expression in a tissue preparation, the term “higher than normal level of IL-1β” refers to that the staining signal generated by specific IL-1β protein or IL-1β RNA detecting molecule is distinguishably stronger than staining signal of the surrounding tissue not expressing IL-1β.

Inflammation component is universally present, albeit to different degrees, in the cancer development. Further cancers include but not limited to haematological malignancies, brain tumors, bone cancer and nose and throat cancer. Haematological malignancies are the types of cancer affecting blood, bone marrow and lymph nodes. They are referred to as leukaemia, lymphoma and myeloma depending on the type of cell affected. Leukemia includes Acute Lymphoblastic Leukemia (adult or childhood), Acute Myeloid Leukemia, (Adult and childhood), Chronic Lymphocytic Leukemia, Chronic Myelogenous Leukemia and Hairy Cell Leukemia. Lymphoma includes AIDS-Related Lymphoma, Cutaneous T-Cell Lymphoma (Mycosis Fungoides and the Sézary Syndrome), Hodgkin Lymphoma (Adult or childhood), Mycosis Fungoides, Non-Hodgkin Lymphoma (Adult or childhood), Primary Central Nervous System Lymphoma, sézary Syndrome, T-Cell Lymphoma, Cutaneous (Mycosis Fungoides and the Sézary Syndrome) and Waldenström Macroglobulinemia (Non-Hodgkin Lymphoma). Other haematological malignancies include Chronic Myeloproliferative Neoplasms, Langerhans Cell Histiocytosis, Multiple Myeloma/Plasma Cell Neoplasm, Myelodysplastic Syndromes and Myelodysplastic/Myeloproliferative Neoplasms.

Primary brain tumors include Anaplastic astrocytomas and glioblastomas, Meningiomas and other mesenchymal tumors, ituitary tumors, Schwannomas, CNS lymphomas, Oligodendrogliomas, Ependymomas, Low-grade astrocytomas, Medulloblastomas. Primary spinal tumors include Schwannomas, meningiomas, and ependymomas, Sarcomas, Astrocytomas, Vascular tumors, Chordomas and Neuroblastoma.

Liver cancer include Hepatocellular carcinoma, Intrahepatic cholangiocarcinoma (bile duct cancer), Angiosarcoma and hemangiosarcoma and Hepatoblastoma.

Nose and throat cancer are known collectively as head and neck cancers usually begin in the squamous cells that line the moist, mucosal surfaces inside the head and neck (for example, inside the mouth, the nose, and the throat). These squamous cell cancers are often referred to as squamous cell carcinomas of the head and neck. Cancers of the head and neck are further categorized by the area of the head or neck in which they begin: Oral cavity, Pharynx, Larynx, Paranasal sinuses and nasal cavity, Salivary glands.

In one embodiment, the present invention provides an IL-1β binding antibody or a functional fragment thereof for use in the treatment and/or prevention of lung cancer, wherein the incidence rate for lung cancer is reduced by at least 30%, at least 40% or at least 50%, in comparison to patients not receiving such treatment.

Lung cancer includes small cell lung cancer and non-small cell lung cancer (NSCLC)/Non-small-cell lung carcinoma (NSCLC). NSCLC is any type of epithelial lung cancer other than small cell lung carcinoma (SCLC) and can be subclassified as squamous (˜30%) or non-squamous (˜70%; includes adenocarcinoma and large cell histologies) histological types. The term “NSCLC” includes but is not limited to adenocarcinoma of the lung (herein referred to as “adenocarcinoma”), poorly differentiated large cell carcinoma, squamous cell (epidermoid) lung carcinoma, adenosquamous carcinoma and sarcomatoid carcinoma and bronchioalveolar carcinoma. Lung cancer also includes metastases to lung and small cell lung cancer. In one embodiment of the invention, the lung cancer is small cell lung cancer. In another embodiment, the lung cancer is NSCLC. In one embodiment the lung cancer is adenocarcinoma of the lung. In another embodiment the lung cancer is poorly differentiated large cell carcinoma in lung. In another embodiment the lung cancer is non-squamous lung cancer. In another embodiment of the invention the lung cancer is squamous cell (epidermoid) lung carcinoma. In yet another embodiment, the lung cancer is selected from the group consisting of adenosquamous carcinoma or sarcomatoid carcinoma or metastases to lung.

As used herein, the terms “treat”, “treatment” and “treating” refer to the reduction or amelioration of the progression, severity and/or duration of a disorder, e.g., a proliferative disorder, or the amelioration of one or more symptoms, suitably of one or more discernible symptoms, of the disorder resulting from the administration of one or more therapies. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count. As far as cancers that have at least a partial inflammatory basis are concerned, taking lung cancer as an example, the term treatment refers to at least one of the following: alleviating one or more symptoms of lung cancer, delaying progression of lung cancer, shrinking tumor size in lung cancer patient, inhibiting lung cancer tumor growth, prolonging overall survival, prolonging progression free survival, preventing or delaying lung cancer tumor metastasis, reducing (such as eradiating) preexisting lung cancer tumor metastasis, reducing incidence or burden of preexisting lung cancer tumor metastasis, or preventing recurrence of lung cancer.

NSCLC is staged according to established guidelines, for example AJCC Cancer Staging Manual. 8th ed. New York: Springer; 2017, summarized by Goldstraw P. et al. The IASLC lung cancer staging project: proposals for revision of the TNM stage groupings in the forthcoming (eighth) edition of the TNM classification for lung cancer. Journal of Thoracic Oncology 2016; 11(1):39-51). Stage I is characterized by a localized tumor, which has not spread to any lymph nodes. Stage II is characterized by a localized tumor, which has spread to a lymph node contained within the surrounding part of the lung. In general, stage I or II are regarded as early stage as they display a size and location amenable for surgical removal.

Stage III is characterized by a localized tumor, which has spread to a regional lymph node not contained within the lung, for example, a mediastinal lymph node. Stage III is further divided into two substages: stage IIIA, in which the lymph node metastasis is on the same side of the lung as the primary tumor, and stage IIIB, in which the cancer has spread to the opposite lung, to a lymph node above the collarbone, to the fluid surrounding the lungs, or in which the cancer grows into a vital structure of the chest. Stage IV is characterized by spreading of the cancer to different sections (lobes) of the lung, or to distant sites within the body, for example, to the brain, the bones, the liver, and/or in the adrenal glands.

In a preferred embodiment, the patient has early stage of lung cancer, especially NSCLC. In a preferred embodiment, the patient has been diagnosed with lung cancer after imaging based lung cancer screening. In another embodiment, the lung cancer is an advanced, metastatic, relapsed, and/or refractory lung cancer. In one embodiment, the patient has stage IA NSCLC. In one embodiment, the patient has stage D3 NSCLC. In one embodiment, the patient has stage IIA NSCLC. In one embodiment, the patient has stage IIB NSCLC. In one embodiment, the patient has stage IIIA NSCLC. In one embodiment, the patient has stage IIIB NSCLC. In a further embodiment, the patient has stage IV NSCLC.

In one embodiment, the patient is a smoker, including current smoker and past smoker. The CANTOS trial data are consistent with the general conception that there is a higher lung cancer incidence among smokers than non-smokers. While both current smoker and past smoker have reduced hazard ratio in the treatment group compared to placebo, stratification by smoking indicated greater relative benefits of canakinumab on lung cancer among current as compared to past smokers (HR 0.50, P=0.005 for current smokers; HR 0.61, P=0.006 for past smokers). In the CANTOS trial specifically current smoker is defined as someone who smoked within the last 30 days at the time of screening. The definition of past smoker is someone who smoked in the past but not within the last 30 days at the time of screening.

Accordingly, in one embodiment, the subject is a smoker. In one further embodiment, the subject is a past smoker. In one embodiment, the present invention provides an IL-1β binding antibody or a functional fragment thereof for use in the treatment and/or prevention of lung cancer, wherein the incidence rate for lung cancer is reduced by at least 30%, at least 40% or at least 50% for smokers as compared to smokers not receiving such treatment.

In one embodiment, the subject is a male patient with lung cancer. In one embodiment said male patient is a current or past smoker.

In one embodiment, the present invention provides the use of an IL-1β binding antibody or a functional fragment thereof, suitably canakinumab or a functional fragment thereof, gevokizumab or a functional fragment thereof, in the treatment and/or prevention of cancer having at least a partial inflammatory basis, including lung cancer, in a patient who has a higher than normal level of C-reactive protein (hsCRP). In one further embodiment, this patient is a smoker. In one further embodiment, this patient is a current smoker. Typically cancers that have at least a partial inflammatory basis include but is not limited lung cancer, especially NSCLC, colorectal cancer (CRC), melanoma, gastric cancer (including esophageal cancer), renal cell carcinoma (RCC), breast cancer, prostate cancer, head and neck cancer, bladder cancer, hepatocellular carcinoma (HCC), ovarian cancer, cervical cancer, endometrial cancer, pancreatic cancer, neuroendocrine cancer, multiple myeloma, acute myeloblastic leukemia (AML), and biliary tract cancer.

A higher than normal level of C-reactive protein (hsCRP) has been particularly reported in, including but not being limited to, lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer.

As used herein, “C-reactive protein” and “CRP” refers to serum or plasma C-reactive protein, which is typically used as an indicator of the acute phase response to inflammation. Nonetheless, CRP level may become elevated in chronic illnesses such as cancer. The level of CRP in serum or plasma may be given in any concentration, e.g., mg/dl, mg/L, nmol/L. Levels of CRP may be measured by a variety of well known methods, e.g., radial immunodiffusion, electroimmunoassay, immunoturbidimetry (e.g., particle (e.g., latex)-enhanced turbidimetric immunoassay), ELISA, turbidimetric methods, fluorescence polarization immunoassay, and laser nephelometry. Testing for CRP may employ a standard CRP test or a high sensitivity CRP (hsCRP) test (i.e., a high sensitivity test that is capable of measuring lower levels of CRP in a sample, e.g., using immunoassay or laser nephelometry). Kits for detecting levels of CRP may be purchased from various companies, e.g., Calbiotech, Inc, Cayman Chemical, Roche Diagnostics Corporation, Abazyme, DADE Behring, Abnova Corporation, Aniara Corporation, Bio-Quant Inc., Siemens Healthcare Diagnostics, Abbott Laboratories etc.

As used herein, the term “hsCRP” refers to the level of CRP in the blood (serum or plasma) as measured by high sensitivity CRP testing. For example, Tina-quant C-reactive protein (latex) high sensitivity assay (Roche Diagnostics Corporation) may be used for quantification of the hsCRP level of a subject. Such latex-enhanced turbidimetric immunoassay may be analysed on the Cobas® platform (Roche Diagnostics Corporation) or Roche/Hitachi (e.g. Modular P) analyzer. In the CANTOS trial the hsCRP level was measured by Tina-quant C-reactive protein (latex) high sensitivity assay (Roche Diagnostics Corporation) on the Roche/Hitachi Modular P analyzer, which can be used typically and preferably as the method in measuring hsCRP level. Alternatively the hsCRP level can be measured by another method, for example by another approved companion diagnostic kit, the value of which can be calibrated against the value measured by the Tina-quant method.

Each local laboratory employ a cutoff value for abnormal (high) CRP or hsCRP based on that laboratory's rule for calculating normal maximum CRP, i.e. based on that laboratory's reference standard. A physician generally orders a CRP test from a local laboratory, and the local laboratory determines CRP or hsCRP value and reports normal or abnormal (low or high) CRP using the rule that particular laboratory employs to calculate normal CRP, namely based on its reference standard. Thus whether a patient has a higher than normal level of C-reactive protein (hsCRP) can be determined by the local laboratory where the test is conducted.

The present invention has shown for the first time in a clinical setting with the tested dosing range, that canakinumab is effective in hazard reduction of total lung cancer and fatal lung cancer. The effect is most pronouncesd in the cohort allocated to the highest canakinumab dose (300 mg twice over a two-week period and then every 3 months).

Furthermore, the present invention has shown for the first time in a clinical setting that an IL-1β antibody, canakinumab, is effective in reducing hsCRP level and the reduction of hsCRP is linked to effects in treating and/or preventing lung cancer. Hence it is plausible that an IL-1β antibody or a fragment thereof, such as canakinumab or gevokizumab, is effective in treating and/or preventing other cancer having at least partially inflammatory basis in a patient, especially when said patient has higher than normal level of hsCRP.

Furthermore, the present invention provides effective dosing ranges, within which the HsCRP level can be reduced to certain threshold, below which more patients with cancer having at least partially inflammatory basis can become responder or below which the same patient can benefit more from the great therapeutic effect of the Drug of the invention with negligible or tolerable side effects.

In one embodiment, the present invention provides the use of an IL-1β binding antibody or a functional fragment thereof, suitably canakinumab or gevokizumab, for the treatment and/or prevention of cancer that has at least a partial inflammatory basis, including lung cancer, in a patient who has high sensitivity C-reactive protein (hsCRP) level equal to or higher than 2 mg/L, equal to or higher than 3 mg/L, equal to or higher than 4 mg/L, equal to or higher than 5 mg/L, equal to or higher than 6 mg/L, equal to or higher than 7 mg/L, equal to or higher than 8 mg/L, equal to or higher than 9 mg/L, equal to or higher than 10 mg/L, equal to or higher than 12 mg/L, equal to or higher than 15 mg/L, equal to or higher than 20 mg/L or equal to or higher than 25 mg/L, preferably before first administration of said IL-1β binding antibody or functional fragment thereof. Preferably said patient has a hsCRP level equal to or higher than 4 mg/L. Preferably said patient has a hsCRP level equal to or higher than 6 mg/L. Preferably said patient has a hsCRP level equal to or higher than 10 mg/L. Preferably said patient has a hsCRP level equal to or higher than 20 mg/L. In one further embodiment, this patient is a smoker. In one further embodiment, this patient is a current smoker.

In one embodiment, the present invention provides the use of an IL-1β binding antibody or a functional fragment thereof, suitably canakinumab or gevokizumab, for the treatment of cancer that has at least a partial inflammatory basis in a patient who has a high sensitivity C-reactive protein (hsCRP) level equal to or higher than 2 mg/L, higher than 6 mg/L, equal to or higher than 10 mg/L or equal to or higher than 20 mg/L, preferably before first administration of DRUG of the invention. In a preferred embodiment cancer that has at least a partial inflammatory basis is selected from a list consisting of lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer.

In one embodiment, the present invention provides the use of an IL-1β binding antibody or a functional fragment thereof, suitably canakinumab or gevokizumab, for the treatment of CRC in a patient who has a high sensitivity C-reactive protein (hsCRP) level equal to or higher than 2 mg/L, higher than 6 mg/L, equal to or higher than 10 mg/L or equal to or higher than 20 mg/L, preferably before first administration of DRUG of the invention.

In one embodiment, the present invention provides the use of an IL-1β binding antibody or a functional fragment thereof, suitably canakinumab or gevokizumab, for the treatment of RCC in a patient who has a high sensitivity C-reactive protein (hsCRP) level equal to or higher than 2 mg/L, higher than 6 mg/L, equal to or higher than 10 mg/L or equal to or higher than 20 mg/L, preferably before first administration of DRUG of the invention.

In one embodiment, the present invention provides the use of an IL-1β binding antibody or a functional fragment thereof, suitably canakinumab or gevokizumab, for the treatment of pancreatic cancer in a patient who has a high sensitivity C-reactive protein (hsCRP) level equal to or higher than 2 mg/L, higher than 6 mg/L, equal to or higher than 10 mg/L or equal to or higher than 20 mg/L, preferably before first administration of DRUG of the invention.

In one embodiment, the present invention provides the use of an IL-1β binding antibody or a functional fragment thereof, suitably canakinumab or gevokizumab, for the treatment of melanoma in a patient who has a high sensitivity C-reactive protein (hsCRP) level equal to or higher than 2 mg/L, higher than 6 mg/L, equal to or higher than 10 mg/L or equal to or higher than 20 mg/L, preferably before first administration of DRUG of the invention.

In one embodiment, the present invention provides the use of an IL-1β binding antibody or a functional fragment thereof, suitably canakinumab or gevokizumab, for the treatment of HCC in a patient who has a high sensitivity C-reactive protein (hsCRP) level equal to or higher than 2 mg/L, higher than 6 mg/L, equal to or higher than 10 mg/L or equal to or higher than 20 mg/L, preferably before first administration of DRUG of the invention.

In one embodiment, the present invention provides the use of an IL-1β binding antibody or a functional fragment thereof, suitably canakinumab or gevokizumab, for the treatment of gastric cancer (including esophageal cancer), in a patient who has a high sensitivity C-reactive protein (hsCRP) level equal to or higher than 2 mg/L, higher than 6 mg/L, equal to or higher than 10 mg/L or equal to or higher than 20 mg/L, preferably before first administration of DRUG of the invention.

In one embodiment, the present invention provide the use of an IL-1β binding antibody or a functional fragment thereof, suitably canakinumab, in the treatment and/or prevention of lung cancer in a patient, wherein said patient has atherosclerosis.

In one embodiment, the present invention provide the use of canakinumab in the treatment and/or prevention of lung cancer in a patient, wherein said patient has suffered from a qualifying CV event.

As used herein, the term “qualifying CV event” is selected from the group comprising myocardial infarction (MI), stroke, unstable angina, revascularization, stent thrombosis, acute coronary syndrome or any other CV event (excluding cardiovascular death) which precedes the start of IL-1β binding antibody or functional fragment thereof therapy.

In one embodiment, the present invention provide the use of canakinumab in the treatment and/or prevention of lung cancer in a patient, wherein said patient has suffered from a previous myocardial infarction. In a further embodiment, said patient is a stable post-myocardial infarction patient.

IL-1β inhibitors include but not be limited to canakinumab or a functional fragment thereof, gevokizumab or a functional fragment thereof, Anakinra, diacerein, Rilonacept, IL-1 Affibody (SOBI 006, Z-FC (Swedish Orphan Biovitrum/Affibody)) and Lutikizumab (ABT-981) (Abbott), CDP-484 (Celltech), LY-2189102 (Lilly).

In one embodiment of any use or method of the invention, said IL-1β binding antibody is canakinumab. Canakinumab (ACZ885) is a high-affinity, fully human monoclonal antibody of the IgG1/k to interleukin-1β, developed for the treatment of IL-1β driven inflammatory diseases. It is designed to bind to human IL-1β and thus blocks the interaction of this cytokine with its receptors. Canakinumab is disclosed in WO02/16436 which is hereby incorporated by reference in its entirety.

In other embodiments of any use or method of the invention, said IL-1β binding antibody is gevokizumab. Gevokizumab (XOMA-052) is a high-affinity, humanized monoclonal antibody of the IgG2 isotype to interleukin-1β, developed for the treatment of IL-10 driven inflammatory diseases. Gevokizumab modulates IL-1β binding to its signaling receptor. Gevokizumab is disclosed in WO2007/002261 which is hereby incorporated by reference in its entirety.

In one embodiment said IL-1β binding antibody is LY-2189102, which is a humanised interleukin-1 beta (IL-1β) monoclonal antibody.

In one embodiment said IL-1β binding antibody or a functional fragment thereof is CDP-484 (Celltech), which is an antibody fragment blocking IL-1β.

In one embodiment said IL-1β binding antibody or a functional fragment thereof is IL-1 Affibody (SOBI 006, Z-FC (Swedish Orphan Biovitrum/Affibody)).

In one embodiment said IL-1β binding antibody or a functional fragment thereof is Lutikizumab (ABT-981) (Abbott), which is a dual-variable domain antibody targeting interleukin 1 alpha (IL-1α) and interleukin 1 beta (IL-1β).

The present invention has shown for the first time in a clinical setting that an IL-1β antibody, canakinumab, is effective in reducing hsCRP level and the reduction of hsCRP is linked to effects in treating and/or preventing lung cancer. If an IL-1β inhibitor, such as an an IL-1β antibody or a functional fragment thereof, is administered in a dose range that can effectively reduce hsCRP level in a patient with cancer having at least partial inflamatory basis, treatment effect of said cancer can possibly be achieved. Dose range, of a particular IL-1β inhibitor, preferably IL-1β antibody or a functional fragment thereof, that can effectively reduce hsCRP level is known or can be tested in a clinical setting.

Thus in one embodiment, the present invention comprises administering the IL-1β binding antibody or a functional fragment thereof to a patient with a cancer that has at least a partial inflammatory basis, including lung cancer, in the range of about 30 mg to about 750 mg per treatment, preferably in the range of about 60 mg to about 400 mg per treatment, alternatively 100 mg-600 mg, 100 mg to 450 mg, 100 mg to 300 mg, alternatively 150 mg-600 mg, 150 mg to 450 mg, 150 mg to 300 mg, preferably 150 mg to 300 mg per treatment; alternatively about 90 mg to about 300 mg, or about 90 mg to about 200 mg per treatment, alternatively at least 150 mg, at least 180 mg, at least 300 mg, at least 250 mg, at least 300 mg per treatment. In one embodiment the patient with a cancer that has at least a partial inflammatory basis, including lung cancer, receives each treatment every 2 weeks, every three weeks, every four weeks (monthly), every 6 weeks, bimonthly (every 2 months) or quarterly (every 3 months). The term “per treatment”, as used in this application and particularly in this context, should be understood as the total amount of drug received per hospital visit or per self administration or per administration helped by a health care giver. Normally and preferably the total amount of drug received per treatment is administered to a patient within one day, preferably within half a day, preferably within 4 hours, preferably within 2 hours. Typically cancers that have at least a partial inflammatory basis include but not limited to lung cancer, especially NSCLC, colorectal cancer, melanoma, gastric cancer (including esophageal cancer), renal cell carcinoma (RCC), breast, hepatocellular carcinoma (HCC), prostate cancer, bladder cancer, AML, multiple myeloma and pancreatic cancer.

In one preferred embodiment patient with cancer that has at least a partial inflammatory basis, including lung cancer, receives a dose of about 90 mg to about 450 mg of the IL-1β binding antibody or a functional fragment thereof per treatment. In one embodiment the patient with cancer that has at least a partial inflammatory basis receives DRUG of the invention monthly. In one embodiment the patient with cancer that has at least a partial inflammatory basis receives DRUG of the invention every three week. In one embodiment the patient with lung cancer receives DRUG of the invention monthly. In one embodiment the patient with lung cancer receives DRUG of the invention every three week. In one embodiment the range of DRUG of the invention is at least 150 mg or at least 200 mg. In one embodiment the range of DRUG of the invention is 180 mg to 450 mg.

In one embodiment said cancer having at least a partial inflammatory basis is breast cancer. In one embodiment said cancer is colorectal cancer. In one embodiment said cancer is gastric cancer. In one embodiment said cancer is RCC. In one embodiment said cancer is melanoma. In one embodiment said cancer is pancreatic cancer.

In practice some times the time interval can not be strictly kept due to the limitation of the availability of doctor, patient or the drug/facility. Thus the time interval can slightly vary, normally between ±5 days, ±4 days, ±3 days, ±2 days or preferably ±1 day.

In one embodiment, the present invention comprises administering the IL-1β binding antibody or a functional fragment thereof to a patient with a cancer having at least a partial inflammatory basis, including lung cancer, in a total dose of from 100 mg to about 750 mg, alternatively 100 mg-600 mg, 100 mg to 450 mg, 100 mg to 300 mg, alternatively in a total dose of from 150 mg-600 mg, 150 mg to 450 mg, 150 mg to 300 mg, alternatively in a total dose of at least 150 mg, at least 180 mg, at least 250 mg, at least 300 mg, over a period of 2 weeks, 3 weeks, 4 weeks, 6 weeks, 8 weeks or 12 weeks, preferably 4 weeks. In one embodiment total dose of DRUG of the invention is 180 mg to 450 mg.

In one embodiment, the total dose of the DRUG of the invention is administered multiple times, preferably 2, 3 or 4 times over the above defined period. In one embodiment, DRUG of the invention is administered once over the above defined period.

Some times it is desirable to quickly reduce inflammation of patients diagnosed with cancer having at least partially inflammatory basis, including lung cancer. IL-1β auto-induction has been shown in human mononuclear blood, human vascular endothelial, and vascular smooth muscle cells in vitro and in rabbits in vivo where IL-1 has been shown to induce its own gene expression and circulating IL-1β level (Dinarello et al. 1987, Warner et al. 1987a, and Warner et al. 1987b).

This induction period over 2 weeks by administration of a first dose followed by a second dose two weeks after administration of the first dose is to assure that auto-induction of IL-1β pathway is adequately inhibited at initiation of treatment. The complete suppression of IL-1β related gene expression achieved with this early high dose administration, coupled with the continuous canakinumab treatment effect which has been proven to last the entire quarterly dosing period used in CANTOS, is to minimize the potential for IL-1β rebound. In addition, data in the setting of acute inflammation suggests that higher initial doses of canakinumab that can be achieved through induction are safe and provide an opportunity to ameliorate concern regarding potential auto-induction of IL-1β and to achieve greater early suppression of IL-1β related gene expression.

Thus in one embodiment, the present invention, while keeping the above described dosing schedules, especially envisages the second administration of DRUG of the invention is at most two weeks, preferably two weeks apart from the first administration. Then the third and the further administration will following the schedule of every 2 weeks, every 3 weeks, every 4 weeks (monthly), every 6 weeks, bimonthly (every 2 months) or quarterly (every 3 months).

In one embodiment, the IL-1β binding antibody is canakinumab, wherein canakinumab is administered to a patient with cancer having at least a partial inflammatory basis, including lung cancer, in the range of about 100 mg to about 750 mg per treatment, alternatively 100 mg-600 mg, 100 mg to 450 mg, 100 mg to 300 mg, alternatively 150 mg-600 mg, 150 mg to 450 mg, 150 mg to 300 mg per treatment, alternatively about 200 mg to 400 mg, 200 mg to 300 mg, alternatively at least 150 mg, at least 200 mg, at least 250 mg, at least 300 mg per treatment. In one embodiment the patient with cancer having at least a partial inflammatory basis, including lung cancer, receives each treatment every 2 weeks, every 3 weeks, every 4 weeks (monthly), every 6 weeks, bimonthly (every 2 months) or quarterly (every 3 months). Typically cancer having at least a partial inflammatory basis includes but not be limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer. In one embodiment the patient with lung cancer receives canakinumab monthly or every three weeks. In one embodiment the preferred dose range of canakinumab is 200 mg to 450 mg, further preferred 300 mg to 450 mg, further preferred 350 mg to 450 mg per treatment. In one embodiment the preferred dose range of canakinumab for patient with lung cancer is 200 mg to 450 mg every 3 weeks or monthly. In one embodiment the preferred dose of canakinumab for patient with lung cancer is 200 mg every 3 weeks. In one embodiment the preferred dose of canakinumab for patient with lung cancer is 200 mg monthly. In one embodiment the patient with cancer that has at least a partial inflammatory basis receives canakinumab monthly or every three week. In one embodiment the patient with cancer that has at least a partial inflammatory basis receives canakinumab in the dose range of 200 mg to 450 mg monthly or every three week. In one embodiment the patient with cancer that has at least a partial inflammatory basis receives canakinumab at a dose of 200 mg monthly or every three weeks.

Suitable the above dose and dosing apply to the use of a functional fragment of canakinumab according to the present invention.

In one embodiment, the present invention comprises administering canakinumab to a patient with cancer that has at least a partial inflammatory basis, including lung cancer, in a total dose of from 100 mg to about 750 mg, alternatively 100 mg-600 mg, 100 mg to 450 mg, 100 mg to 300 mg, alternatively 150 mg-600 mg, 150 mg to 450 mg, 150 mg to 300 mg, preferably 150 mg to 300 mg, preferably 300 mg to 450 mg; alternatively at least 150 mg, at least 200 mg, at least 250 mg, at least 300 mg, perably at least 300 mg, over a period of 2 weeks, 3 weeks, 4 weeks, 6 weeks, 8 weeks or 12 weeks, preferably 4 weeks. In one embodiment, canakinumab is administered multiple times, preferably 2, 3 or 4 times over the above defined period. In one embodiment, canakinumab is administered once over the above defined period. In one embodiment the preferred total dose of canakinumab is 200 mg to 450 mg, further preferred 300 mg to 450 mg, further preferred 350 mg to 450 mg.

In one embodiment, the present invention, while keeping the above described dosing schedules, especially envisages the second administration of canakinumab is at most two weeks, preferably two weeks apart from the first administration.

In one embodiment, the present invention comprises administering canakinumab at a dose of 150 mg every 2 weeks, every 3 weeks or monthly.

In one embodiment, the present invention comprises administering canakinumab at a dose of 300 mg every 2 weeks, every 3 weeks, monthly, every 6 weeks, bimonthly (every 2 months) or quarterly (every 3 months).

In one embodiment, the present invention comprises administering canakinumab at a dose of 300 mg once per month (monthly). In one further embodiment, the present invention, while keeping the above described dosing schedules, especially envisages the second administration of canakinumab at 300 mg is at most two weeks, preferably two weeks apart from the first administration.

In one embodiment of the invention, canakinumab is administered to a patient in need at 300 mg twice over a two week period and then every 3 month.

In one embodiment said cancer having at least a partial inflammatory basis is breast cancer. In one embodiment said cancer is correlectal cancer. In one embodiment said cancer is gastric cancer. In one embodiment said cancer is renal carcinoma. In one embodiment said cancer is melanoma.

In one embodiment, the present invention comprises administering gevokizumab to a patient with cancer that has at least a partial inflammatory basis, including lung cancer, in the range of about 30 mg to about 450 mg per treatment, alternatively 90 mg-450 mg, 90 mg to 360 mg, 90 mg to 270 mg, 90 mg to 180 mg per treatment; alternatively 120 mg-450 mg, 120 mg to 360 mg, 120 mg to 270 mg, 120 mg to 180 mg per treatment, alternatively 150 mg-450 mg, 150 mg to 360 mg, 150 mg to 270 mg, 150 mg to 180 mg per treatment, alternatively 180 mg-450 mg, 180 mg to 360 mg, 180 mg to 270 mg per treatment; alternatively about 60 mg to about 360 mg, about 60 mg to 180 mg per treatment; alternatively at least 150 mg, at least 180 mg, at least 240 mg, at least 270 mg per treatment. In one embodiment the patient with cancer that has at least a partial inflammatory basis, including lung cancer, receives treatment every 2 weeks, every 3 weeks, monthly (every 4 weeks), every 6 weeks, bimonthly (every 2 months) or quarterly (every 3 months). In one embodiment the patient with cancer that has at least a partial inflammatory basis, including lung cancer, receives at least one, preferably one treatment per month. Typically cancers that have at least a partial inflammatory basis include but not limited to lung cancer, especially NSCLC, colorectal cancer, melanoma, gastric cancer (including esophageal cancer), renal cell carcinoma (RCC), breast, hepatocellular carcinoma (HCC), prostate cancer, bladder cancer, AML, multiple myeloma and pancreatic cancer. In one embodiment the preferred range of gevokizumab is 150 mg to 270 mg. In one embodiment the preferred range of gevokizumab is 60 mg to 180 mg, further preferred 60 mg to 90 mg. In one embodiment the preferred range of gevokizumab is 90 mg to 270 mg, further preferred 90 mg to 180 mg. In one embodiment the preferred schedule is every 3 weeks or monthly. In one embodiment the patient receives gevokizumab 60 mg to 90 mg every 3 weeks. In one embodiment the patient receives gevokizumab 60 mg to 90 mg monthly. In one embodiment the patient with cancer that has at least a partial inflammatory basis receives gevokizumab about 90 mg to about 360 mg, 90 mg to about 270 mg, 120 mg to 270 mg, 90 mg to 180 mg, 120 mg to 180 mg, 120 mg or 90 mg every 3 weeks. In one embodiment the patient with cancer that has at least a partial inflammatory basis receives gevokizumab about 90 mg to about 360 mg, 90 mg to about 270 mg, 120 mg to 270 mg, 90 mg to 180 mg, 120 mg to 180 mg, 120 mg or 90 mg monthly.

In one embodiment the patient with cancer that has at least a partial inflammatory basis receives gevokizumab about 120 mg every 3 weeks. In one embodiment the patient receives gevokizumab about 120 mg monthly. In one embodiment the patient with cancer that has at least a partial inflammatory basis receives gevokizumab about 90 mg every 3 weeks. In one embodiment the patient receives gevokizumab about 90 mg monthly. In one embodiment the patient with cancer that has at least a partial inflammatory basis receives gevokizumab about 180 mg every 3 weeks. In one embodiment the patient receives gevokizumab about 180 mg monthly. In one embodiment the patient with cancer that has at least a partial inflammatory basis receives gevokizumab about 200 mg every 3 weeks. In one embodiment the patient receives gevokizumab about 200 mg monthly.

Suitable the above dose and dosing apply to the use of a functional fragment of gevokizumab according to the present invention.

In one embodiment, the present invention comprises administering gevokizumab to a patient with lung cancer in a total dose of 90 mg-450 mg, 90 mg to 360 mg, 90 mg to 270 mg, 90 mg to 180 mg, alternatively 120 mg-450 mg, 120 mg to 360 mg, 120 mg to 270 mg, 120 mg to 180 mg, alternatively 150 mg-450 mg, 150 mg to 360 mg, 150 mg to 270 mg, 150 mg to 180 mg, alternatively 180 mg-450 mg, 180 mg to 360 mg, 180 mg to 270 mg, alternatively at least 90 mg, at least 120 mg, at least 150 mg, at least 180 mg over a period of 2 weeks, 3 weeks, 4 weeks, 6 weeks, 8 weeks or 12 weeks, preferably 4 weeks. In one embodiment, gevokizumab is administered multiple times, preferably 2, 3 or 4 times over the above defined period. In one embodiment, gevokizumab is administered once over the above defined period. In one embodiment the preferred total dose of gevokizumab is 180 mg to 360 mg. In one embodiment, the patient with lung cancer receives gevokizumab at least one, preferably one treatment per month.

In one embodiment, the present invention, while keeping the above described dosing schedules, especially envisages the second administration of gevokizumab is at most two weeks, preferably two weeks apart from the first administration.

In one embodiment, the present invention comprises administering gevokizumab at a dose of 60 mg every 2 weeks, every 3 weeks or monthly.

In one embodiment, the present invention comprises administering gevokizumab at a dose of 90 mg every 2 weeks, every 3 weeks or monthly.

In one embodiment, the present invention comprises administering gevokizumab at a dose of 180 mg every 2 weeks, every 3 weeks (±3 days), monthly, every 6 weeks, bimonthly (every 2 months) or quarterly (every 3 months).

In one embodiment, the present invention comprises administering gevokizumab at a dose of 180 mg once per month (monthly). In one further embodiment, the present invention, while keeping the above described dosing schedules, envisages the second administration of gevokizumab at 180 mg is at most two weeks, preferably two weeks apart from the first administration.

In one embodiment said cancer having at least a partial inflammatory basis is breast cancer. In one embodiment said cancer is colorectal cancer. In one embodiment said cancer is gastric cancer. In one embodiment said cancer is renal carcinoma. In one embodiment said cancer is melanoma.

In one embodiment, the present invention provides an IL-1β binding antibody or a functional fragment thereof, suitably canakinumab, for use in the treatment and/or prevention of cancer that has at least a partial inflammatory basis, including lung cancer, wherein the risk for cancer that has at least a partial inflammatory basis, including lung cancer, is reduced by at least 30%, at least 40%, at least 50% at 3 months from the first administration compared to patient not receiving the treatment. In one preferred embodiment, the dose of the first administration is at 300 mg. In one further preferred embodiment, the dose of the first administration is at 300 mg followed by a second dose of 300 mg within a two-week period. Preferably the result is achieved with a dose of 200 mg canakinumab administered every 3 weeks. Preferably the result is achieved with a dose of 200 mg canakinumab administered every month.

In one embodiment, the present invention provides an IL-1β binding antibody or functional fragment thereof, suitably canakinumab, for use in the treatment and/or prevention of cancer that has at least a partial inflammatory basis, including lung cancer, wherein the risk for lung cancer mortality is reduced by at least 30%, at least 40% or at least 50% compared to a patient not receiving the treatment. Preferably the results is achieved at a dose of 200 mg canakinumab administered every 3 weeks or 300 mg canakinumab administered monthly, preferably for at least for one year, preferably up to 3 years.

In one embodiment, the present invention provides an IL-1β binding antibody or functional fragment thereof, suitably canakinumab, for use in the treatment and/or prevention of lung cancer, wherein the incident rate for adenocarcinoma or poorly differentiatied large cell carcinoma is reduced by at least 30%, at least 40% or at least 50% compared to patient not receiving such treatment. Preferably the results is achieved at a dose of 300 mg of canakinumab monthly administration or preferably at a dose of 200 mg canakinumab administered every 3 weeks or monthly, preferably for at least for one year, preferably up to 3 years.

In one embodiment, the present invention provides an IL-1β binding antibody or functional fragment thereof, suitably canakinumab, for use in the treatment and/or prevention of cancer, wherein the risk for total cancer mortality is reduced by at least 30%, at least 40%, or at least 50% compared to a patient not receiving such treatment. Preferably the results is achieved at a dose of 300 mg or 200 mg canakinumab administered monthly or preferably at a dose of 200 mg canakinumab administered every 3 weeks, preferably subcutaneously, preferably for at least for one year, preferably up to 3 years.

In one embodiment, the present invention provides an IL-1β binding antibody or functional fragment thereof, suitably canakinumab or a functional fragment thereof, suitably gevokizumab or a functional fragment thereof for use, in the treatment of cancer that has at least a partial inflammatory basis, wherein the risk for said cancer mortality is reduced by at least 30%, at least 40% or at least 50% compared to a patient not receiving the treatment. Preferably the results is achieved at a dose of 200 mg canakinumab administered every 3 weeks or monthly, preferably for at least for one year, preferably up to 3 years. Preferably the results is achieved at a dose of 120 mg gevokizumab administered every 3 weeks or monthly, preferably for at least for one year, preferably up to 3 years. Preferably the results is achieved at a dose of 90 mg gevokizumab administered every 3 weeks or monthly, preferably for at least for one year, preferably up to 3 years.

In one embodiment, the present invention provides canakinumab for use in the treatment and/or prevention of lung cancer, wherein the effects were dose dependent with relative hazard reductions of 67% and 77% for total lung cancer and fatal lung cancer, respectively, among those randomly allocated to the highest canakinumab dose (300 mg twice over a two-week period and then every 3 months).

In one embodiment, the present invention provides canakinumab for use in the treatment and/or prevention of lung cancer, wherein beneficial effects of canakinumab are observed on incident lung cancers within weeks from the first administration. In one preferred embodiment, the dose of the first administration is at 300 mg. In one further preferred embodiment, the dose of the first administration is at 300 mg followed by a second dose of 300 mg within a two-week period. In one further preferred embodiment, a dose of 200 mg canakinumab is administered every three weeks or monthly.

In one aspect the present invention provides an IL-1β binding antibody or a functional fragment thereof for use in the treatment of cancer having at least a partial inflammatory basis, including lung cancer, especially NSCLC, in a patient, wherein the efficacy of the treatment correlates with the reduction of hsCRP in said patient, comparing to prior treatment. In one embodiment the present invention provides an IL-1β binding antibody or a functional fragment thereof for use in the treatment of cancer having at least a partial inflammatory basis, including lung cancer, especially NSCLC, in a patient, wherein the CRP level, more precisely the hsCRP level, of said patient has reduced to to below 15 mg/L, below 10 mg/L, preferably to below 6 mg/L, preferably to below 4 mg/L, preferably to below 3 mg/L, preferably to below 2.3 mg/L, preferably to below 2 mg/L, to below 1.8 mg/L, about 6 months, or preferably about 3 months from the first administration of said IL-1β binding antibody or a functional fragment thereof at a proper dose, preferably according to the dosing regimen of the present invention. Typically cancers that have at least a partial inflammatory basis include but not limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer.

In one embodiment, said IL-1β binding antibody is canakinumab or a functional fragment thereof. In one preferred embodiment, the proper dose of the first administration of canakinumab is 300 mg. In one preferred embodiment, canakinumab is administered at a dose of 300 mg monthly. In one preferred embodiment, canakinumab is administered at a dose of 300 mg monthly with an additional dose at 2 weeks interval from the first administration. In one preferred embodiment, canakinumab is administered at a dose of 200 mg. In one preferred embodiment, canakinumab is administered at a dose of 200 mg every 3 weeks or monthly. In one preferred embodiment, canakinumab is administered at a dose of 200 mg every 3 weeks or monthly subcutaneouly.

In one embodiment, said IL-1β binding antibody is gevokizumab or a functional fragment thereof. In one preferred embodiment, the proper dose of the first administration of gevokizumab is 180 mg. In one preferred embodiment, gevokizumab is administered at a dose of 60 mg to 90 mg. In one preferred embodiment, gevokizumab is administered at a dose of 60 mg to 90 mg every 3 weeks or monthly. In one preferred embodiment, gevokizumab is administered at a dose of 120 mg every 3 weeks or every 4 weeks (monthly). In one preferred embodiment, gevokizumab is administered intravenously. In one preferred embodiment, gevokizumab is administered at a dose of 90 mg every 3 weeks or every 4 weeks (monthly) intravenously. In one embodiment the patient with cancer that has at least a partial inflammatory basis receives gevokizumab about 120 mg every 3 weeks. In one embodiment the patient with cancer that has at least a partial inflammatory basis receives gevokizumab about 180 mg every 3 weeks. In one embodiment the patient receives gevokizumab about 180 mg monthly. In one embodiment the patient with cancer that has at least a partial inflammatory basis receives gevokizumab about 200 mg every 3 weeks. In one embodiment the patient receives gevokizumab about 200 mg monthly. Gevokizumab is administered subcutaneously or preferably introvenously.

Further preferably the hsCRP level, of said patient has reduced to below 10 mg/L, preferably to below 6 mg/L, preferably to below 4 mg/L, preferably to below 3 mg/L, preferably to below 2.3 mg/L, preferably to below 2 mg/L, to below 1.8 mg/L, after the first administration of the DRUG of the invention according to the dose regimen of the present invention. In one preferred embodiment, the proper dose of the first administration of canakinumab is at least 150 mg, preferably at least 200 mg. In one preferred embodiment, the proper dose of the first administration of gevokizumab is 90 mg. In one preferred embodiment, the proper dose of the first administration of gevokizumab is 120 mg. In one preferred embodiment, the proper dose of the first administration of gevokizumab is 180 mg. In one preferred embodiment, the proper dose of the first administration of gevokizumab is 200 mg.

In one embodiment said cancer having at least a partial inflammatory basis is breast cancer. In one embodiment said cancer is colorectal cancer. In one embodiment said cancer is gastric cancer. In one embodiment said cancer is renal carcinoma. In one embodiment said cancer is melanoma.

In one aspect the present invention provides an IL-1β binding antibody or a functional fragment thereof for use in the treatment of cancers that have at least a partial inflammatory basis, including lung cancer, especially NSCLC, in a patient, wherein the hsCRP level of said patient has reduced by at least 15%, at least 20%, at least 30% or at least 40% 6 months, or preferably 3 month from the first administration of said IL-1β binding antibody or a functional fragment thereof at a proper dose, preferably according to the dosing regimen of the present invention, as compared to the hsCRP level just prior to the first administration of the IL-1β binding antibody or a functional fragment thereof. Further preferably the hsCRP level of said patient has reduced by at least 15%, at least 20%, at least 30% after the first administration of the DRUG of the invention according to the dose regimen of the present invention.

In one aspect the present invention provides an IL-1β binding antibody or a functional fragment thereof for use in the treatment of cancers that have at least a partial inflammatory basis, including lung cancer, especially NSCLC, in a patient, wherein the IL-6 level of said patient has reduced by at least 15%, at least 20%, at least 30% or at least 40% about 6 months, or preferably about 3 months from the first administration of said IL-1β binding antibody or a functional fragment thereof at a proper dose, preferably according to the dosing regimen of the present invention, as compared to the IL-6 level just prior to the first administration. The term “about” used herein includes a variation of ±10 days from the 3 months or a variation of ±15 days from the 6 months. Typically cancers that have at least a partial inflammatory basis include but not be limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer. In one embodiment, said IL-1β binding antibody is canakinumab or a functional fragment thereof. In one preferred embodiment, the proper dose of the first administration of canakinumab is 300 mg. In one preferred embodiment, canakinumab is administered at a dose of 300 mg monthly. In one preferred embodiment, canakinumab is administered at a dose of 300 mg monthly with an additional dose at 2 weeks from the first administration. In one preferred embodiment, canakinumab is administered at a dose of 200 mg. In one preferred embodiment, canakinumab is administered at a dose of 200 mg every 3 weeks or monthly. In one preferred embodiment, canakinumab is administered at a dose of 200 mg every 3 weeks or monthly subcutaneouly. In another embodiment, said IL-1β binding antibody is gevokizumab or a functional fragment thereof. In one preferred embodiment, the proper dose of the first administration of gevokizumab is 180 mg. In one preferred embodiment, gevokizumab is administered at a dose of 60 mg to 90 mg. In one preferred embodiment, gevokizumab is administered at a dose of 60 mg to 90 mg every 3 weeks or monthly. In one preferred embodiment, gevokizumab is administered at a dose of 120 mg every 3 weeks or every 4 weeks (monthly). In one preferred embodiment, gevokizumab is administered intravenously. In one preferred embodiment, gevokizumab is administered at a dose of 120 mg every 3 weeks or every 4 weeks (monthly) intravenously. In one preferred embodiment, gevokizumab is administered at a dose of 90 mg every 3 weeks or every 4 weeks (monthly) intravenously.

The reduction of the level of hsCRP and the reduction of the level of IL-6 can be used separately or in combination to indicate the efficacy of the treatment or as prognostic markers.

In one embodiment said cancer having at least a partial inflammatory basis is breast cancer. In one embodiment said cancer is correlectal cancer. In one embodiment said cancer is gastric cancer. In one embodiment said cancer is renal carcinoma. In one embodiment said cancer is melanoma.

In one aspect, the present invention provides an IL-1β binding antibody or a functional fragment thereof for use in the treatment and/or prevention of cancers that have at least a partial inflammatory basis, including lung cancer, especially NSCLC, in a patient with a high sensitive C-reactive protein (hsCRP) of ≥2 mg/L, wherein the antibody is canakinumab and the patient experiences a reduced chance of death from cancer over at least a five year period. In one further embodiment the patient has at least a 51% reduced chance of death from cancer over at least a five year period.

In one aspect the present invention provides the use of an IL-1β binding antibody or a functional fragment thereof in the prevention of lung cancer in a patient. The term “prevent”, “preventing” or “prevention” as used herein means the prevention or delay the occurrence of lung cancer in a subject who is otherwise at high risk of developing lung cancer. In one preferred embodiment, canakinumab is administered at a dose of 200 mg. In one preferred embodiment, canakinumab is administered at a dose of 100 mg to 200 mg, preferably 200 mg, every three weeks, monthly, every 6 weeks, every other month or quarterly, preferably subcutaneously. In another embodiment, said IL-1β binding antibody is gevokizumab or a functional fragment thereof. In one preferred embodiment, gevokizumab is administered at a dose of 30 mg to 90 mg. In one preferred embodiment, gevokizumab is administered at a dose of 30 mg to 90 mg every three weeks, monthly, every 6 weeks, every other month or quarterly. In one preferred embodiment, gevokizumab is administered at a dose of 60 mg to 120 mg every three weeks, monthly, every 6 weeks, every other month or quaterly, prefearbly intravenously. In one preferred embodiment, gevokizumab is administered at a dose of 90 mg every three weeks, monthly, every 6 weeks, every other month or quaterly, prefearbly intravenously. In one preferred embodiment, gevokizumab is administered at a dose of 120 mg every three weeks, monthly, every 6 weeks, every other month or quaterly, prefearbly subcutaneously.

Risk factors include but are not limited to age, genetic mutation, smoking, long term exposure to inhalable hazards, for example due to profession, etc.

In one embodient said patient is over 60 years old, over 62 years old or over 65 years or over 70 years old. In one embodiment, said patient is a male. In another embodiment, said patient is female. In one embodiment said patient is a smoker, especially a current smoker. Smoker can be understood, more broadly than the definition of the CANTOS trial, as someone who smokes more than 5 cigarettes a day (current smoker) or someone who has a smoking history (past smoker). Normally the smoking history is in total more than 5 years or more than 10 years. Normally during the smoking period more than 10 cigarettes or more than 20 cigarettes were smoked per day.

In one embodiment said patient has chronic bronchitis. In one embodiment said patient was exposed or has been exposed or is being exposed for long period (more than 5 years or even more than 10 years), for example due to profession, to external inhaled toxins, such as asbestos, silica, smoking, and other external inhaled toxins. If a patient has the above mentioned one, or the combination of any of the two, any of the three, any of the four, any of the five or any of the six conditions, such patient is likely to have higher likelihood of developing lung cancer. The present invention envisages the use of an IL-1β binding antibody or functional fragment thereof, suitably canakinumab or a functional fragment thereof, or gevokizumab or a functional fragment thereof, in the prevention of lung cancer in such a patient. In one preferred embodiment, such a male patient is over 65, or over 70 years old who is a smoker. In one embodiment, such a male patient is over 65 years of age, or over 70 years of age who is a current or past smoker. In one embodiment, such a female patient is over 65 years of age, or over 70 years of age who is a smoker. In one further embodiment, said patient smokes or had smoked in the past more than 10, more than 20 cigarettes or more than 30 cigarettes or more than 40 cigarettes per day.

In one embodiment, the present invention provides an IL-1β binding antibody or a functional fragment thereof, suitably canakinumab, or a functional fragment thereof, or gevokizumab, or a functional fragment thereof, for use in the prevention of lung cancer in a subject with a high sensitive C-reactive protein (hsCRP) equal to or higher than 2 mg/L, or equal to or higher than 3 mg/L, or equal to or higher than 4 mg/L, or equal to or higher than 5 mg/L, equal to or higher than 6 mg/L, equal to or higher than 8 mg/L, equal to or higher than 9 mg/L, or equal to or higher than 10 mg/L as assessed prior to the administration of the IL-1β binding antibody or functional fragment thereof. In one preferred embodiment, said subject has hsCRP level equal to or higher than 6 mg/L as assessed prior to the administration of the IL-1β binding antibody or functional fragment thereof. In one preferred embodiment, said subject had hsCRP level equal to or higher than 10 mg/L as assessed prior to the administration of the IL-1β binding antibody or functional fragment thereof. In one embodiment, said an IL-1β binding antibody is canakinumab or a functional fragment thereof, or gevokizumab or a functional fragment thereof. In one further embodiment, said subject is a smoker. In one further embodiment said subject is over 65 years old. In one further embodiment said subject has inhaled toxins, such as asbestos, silica or smoking for more than 10 years.

In one embodiment, the present invention provides an IL-1β binding antibody or a functional fragment thereof, suitably canakinumab or a functional fragment thereof, or gevokizumab or a functional fragment thereof, for use in the prevention of recurrence or relapse of cancer having at least a partial inflammatory basis, including lung cancer, in a subject, wherein said subject had cancer or lung cancer, which has been surgically removed (resected). Typically cancers that have at least a partial inflammatory basis include but not be limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, multiple myeloma and pancreatic cancer. In a preferred embodiment said patient has completed post-surgery standard chemotherapy (other than the treatment of DRUG of the invention) treatment and/or completed standard radiotherapy treatment. The term post-surgery standard chemotherapy including standard small molecule chemotherapeutic agents and/or antibodies, particularly check point inhibitors. In one further preferred embodiment, canakinumab or gevokizumab is administered as monotherapy in the prevention of recurrence or relapse of cancer having at least a partial inflammatory basis, including lung cancer. In one embodiment, canakinumab or gevokizumab is administered to said patient post-surgery in combination with radiotherapy or in combination with chemotherapy, particularly standard chemotherapy. In one embodiment, canakinumab is administered every month at a dose of 200 mg, particularly when administered as monotherapy, preferably subcutaneously. In one embodiment, canakinumab is administered every 3 weeks or monthly at a dose of 200 mg, particularly when administered in combination with chemotherapy, particularly standard of care chemotherapy, particular in combination with a checkpoint inhibitor, such as a PD-1 or PD-L1 inhibitor, preferably subcutaneously. In one embodiment, gevokizumab is administered every month at a dose of 60 mg to 180 mg, every month at a dose of 90 mg to 120 mg, or 60 mg to 90 mg, preferably 120 mg, particularly when administered as monotherapy in the prevention of recurrence or relapse of cancer having at least a partial inflammatory basis, including lung cancer or colorectal cancer, RCC or gastric cancer, preferably intravenously. In one embodiment, gevokizumab is administered every 3 weeks at a dose of 60 mg to 180 mg, 90 mg to 120 mg or 60 mg to 90 mg, preferably 120 mg, particularly when administered in combination with chemotherapy, particularly standard chemotherapy, particular in combination with a checkpoint inhibitor, such as a PD-1 or PD-L1 inhibitor, preferably intravenously.

In one embodiment said cancer having at least a partial inflammatory basis is breast cancer. In one embodiment said cancer is colorectal cancer. In one embodiment said cancer is gastric cancer. In one embodiment said cancer is renal carcinoma. In one embodiment said cancer is melanoma.

In one embodiment, canakinumab is administered every 3 months at a dose of 50 mg-300 mg, 50-150 mg, 75 mg-150 mg, 100 mg-150 mg, 50 mg, 150 mg or 300 mg. In the aspect of prevention, canakinumab is administered to a patient in need thereof at a dose of 50 mg, 150 mg or 300 mg, preferably 150 mg, monthly, bimonthly or every 3 months. In one embodiment, canakinumab is administered to a patient in need thereof for the prevention of lung cancer at a dose of 150 mg every 3 months.

In one embodiment said gevokizumab is administered every 3 months at a dose of 30 mg-180 mg, 30 mg-120 mg, 30 mg-90 mg, 60 mg-120 mg, 60 mg-90 mg, 30 mg, 60 mg, 90 mg or 180 mg.

In one embodiment, the IL-1β binding antibody or a functional fragment thereof, suitably canakinumab or gevokizumab, is administered to said patient with cancer having at least partial inflammatory basis prior to surgery (neoadjuvant chemotherapy) or post surgery (adjuvant chemotherapy). In one embodiment, IL-1β binding antibody or functional fragment thereof is administered to said patient prior to, concomitantly with or post radiotherapy.

In one aspect, the present invention provides an IL-1β binding antibody or a functional fragment thereof, suitably canakinumab or gevokizumab, for use in a patient in need thereof in the treatment of a cancer having at least partial inflammatory basis, wherein said IL-1β binding antibody or a functional fragment thereof is administered in combination with one or more chemotherapeutic agents. Typically cancer having at least partial inflammatory basis include but not limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer.

In one embodiment the IL-1β binding antibody or a functional fragment thereof, suitably canakinumab or gevokizumab, is administered in combination with one or more chemotherapeutic agents.

Without wishing being bound by the theory, it is believed that typical cancer development requires two steps. Firstly gene alteration results in cell growth and proliferation no longer subject to regulation. Secondly the abnormal tumor cells evade surveillence of the immunue system. Inflammation plays important role in the second step. Therefore, control of inflamation, as supported for the first time by the clinical data from the CANTOS trial, can stop cancer development at the early or earlier stage. Thus it is expected that blocking IL-1β pathway to reduce inflammation would have a general benefit, particularly improvement of the treatment efficacy on top of the standare of care, which is mainly to directly inhibit the growth and prolifration of the maligant cells. In one embodiment the one or more chemotherapeutic agents is the standard of care agents of said cancer having at least partial inflammatory basis.

Check point inhibitors de-supress the immune system through a mechanism different from IL-1β inhibitors. Thus the addition of IL-1β inhibitors, particularly IL-1β binding antibodies or a functional fragment thereof to the standard Check point inhibitors therapy will further active the immune response, particularly at the tumor microenvironment.

In one embodiment, the one or more chemotherapeutic agents is nivolumab and ipilimumab.

In one embodiment, the one or more chemotherapeutic agents is cabozantinib, or a pharmaceutically acceptable salt thereof.

In one embodiment the or more chemotherapeutic agent is Atezolizumab plus bevacizumab.

In one embodiment the one or more chemotherapeutic agent is FOLFIRI plus bevacizumab or FOLFOX plus bevacizumab.

Chemotherapeutic agents are cytotoxic and/or cytostatic drugs (drugs that kill malignant cells, or inhibit their proliferation, respectively) as well as check point inhibitors. Commonly known chemotherapeutic agent includes but is not limited to platinum agents (e.g., cisplatin, carboplatin, oxaliplatin, nedaplatin, triplatin, lipoplatin, satraplatin, picoplatin), antimetabolites (e.g., methotrexate, 5-Fluorouracil, gemcitabine, pemetrexed, edatrexate), mitotic inhibitors (e.g., paclitaxel, albumin-bound paclitaxel, docetaxel, taxotere, docecad), alkylating agents (e.g., cyclophosphamide, mechlorethamine hydrochloride, ifosfamide, melphalan, thiotepa), vinca alkaloids (e.g., vinblastine, vincristine, vindesine, vinorelbine), topoisomerase inhibitors (e.g., etoposide, teniposide, topotecan, irinotecan, camptothecin, doxorubicin), antitumor antibiotics (e.g., mitomycin C) and/or hormone-modulating agents (e.g., anastrozole, tamoxifen). Examples of anti-cancer agents used for chemotherapy include Cyclophosphamide (Cytoxan®), Methotrexate, 5-Fluorouracil (5-FU), Doxorubicin (Adriamycin®), Prednisone, Tamoxifen (Nolvadex®), Paclitaxel (Taxol®), Albumin-bound paclitaxel (nab-paclitaxel, Abraxane®), Leucovorin, Thiotepa (Thioplex®), Anastrozole (Arimidex®), Docetaxel (Taxotere®), Vinorelbine (Navelbine®), Gemcitabine (Gemzar®), Ifosfamide (Ifex®), Pemetrexed (Alimta®), Topotecan, Melphalan (L-Pam®), Cisplatin (Cisplatinum®, Platinol®), Carboplatin (Paraplatin®), Oxaliplatin (Eloxatin®), Nedaplatin (Aqupla®), Triplatin, Lipoplatin (Nanoplatin®), Satraplatin, Picoplatin, Carmustine (BCNU; BiCNU®), Methotrexate (Folex®, Mexate®), Edatrexate, Mitomycin C (Mutamycin®), Mitoxantrone (Novantrone®), Vincristine (Oncovin®), Vinblastine (Velban®), Vinorelbine (Navelbine®), Vindesine (Eldisine®), Fenretinide, Topotecan, Irinotecan (Camptosar®), 9-amino-camptothecin [9-AC], Biantrazole, Losoxantrone, Etoposide, and Teniposide.

In one embodiment, the preferred combination partner for the IL-1β binding antibody or a functional fragment thereof is a mitotic inhibitor, preferably docetaxel. In one embodiment, the preferred combination partner for canakinumab is a mitotic inhibitor, preferably docetaxel. In one embodiment, the preferred combination partner for gevokizumab is a mitotic inhibitor, preferably docetaxel. In one embodiment said combination is used for the treatment of lung cancer, especially NSCLC.

In one embodiment, the preferred combination partner for the IL-1β binding antibody or a functional fragment thereof is a platinum agent, preferably cisplatin. In one embodiment, the preferred combination partner for canakinumab is a platinum agent, preferably cisplatin. In one embodiment, the preferred combination partner for gevokizumab is a platinum agent, preferably cisplatin. In one embodiment, the one or more chemotherapeutic agent is a platinum-based doublet chemotherapy (PT-DC).

Chemotherapy may comprise the administration of a single anti-cancer agent (drug) or the administration of a combination of anti-cancer agents (drugs), for example, one of the following, commonly administered combinations of: carboplatin and taxol; gemcitabine and cisplatin; gemcitabine and vinorelbine; gemcitabine and paclitaxel; cisplatin and vinorelbine; cisplatin and gemcitabine; cisplatin and paclitaxel (Taxol); cisplatin and docetaxel (Taxotere); cisplatin and etoposide; cisplatin and pemetrexed; carboplatin and vinorelbine; carboplatin and gemcitabine; carboplatin and paclitaxel (Taxol); carboplatin and docetaxel (Taxotere); carboplatin and etoposide; carboplatin and pemetrexed. In one embodiment, the one or more chemotherapeutic agent is a platinum-based doublet chemotherapy (PT-DC).

Another class of chemotherapeutic agents are the inhibitors, especially tyrosine kinase inhibitors, that specifically target growth promoting receptors, especially VEGF-R, EGFR, PFGF-R and ALK, or their downstream members of the signalling transduction pathway, the mutation or overproduction of which results in or contributes to the oncogenesis of the tumor at the site (targeted therapies). Exemplary of targeted therapies drugs approved by the Food and Drug administration (FDA) for the targeted treatment of lung cancer include but not limited bevacizumab (Avastin®), crizotinib (Xalkori®), erlotinib (Tarceva®), gefitinib (Iressa®), afatinib dimaleate (Gilotrif®), ceritinib (LDK378/Zykadia™), everolimus (Afinitor®), ramucirumab (Cyramza®), osimertinib (Tagrisso™), necitumumab (Portrazza™), alectinib (Alecensa®), atezolizumab (Tecentriq™), brigatinib (Alunbrig™), trametinib (Mekinist®), dabrafenib (Tafinlar®), sunitinib (Sutent®) and cetuximab (Erbitux®).

In one embodiment the one or more chemotherapeutic agent to be combined with the IL-1β binding antibody or fragment thereof, suitably canakinumab or gevokizumab, is the agent that is the standard of care agent for lung cancer, including NSCLC and SCLC. Standard of care, can be found, for example from American Society of Clinical Oncology (ASCO) guideline on the systemic treatment of patients with stage IV non-small-cell lung cancer (NSCLC) or American Society of Clinical Oncology (ASCO) guideline on Adjuvant Chemotherapy and Adjuvant Radiation Therapy for Stages I-IIIA Resectable Non-Small Cell Lung Cancer.

In one embodiment the one or more chemotherapeutic agent to be combined with the IL-1β binding antibody or fragment thereof, suitably canakinumab or gevokizumab, is a platinum containing agent or a platinum-based doublet chemotherapy (PT-DC). In one embodiment said combination is used for the treatment of lung cancer, especially NSCLC. In one embodiment one or more chemotherapeutic agent is a tyrosine kinase inhibitor. In one preferred embodiment said tyrosine kinase inhibitor is a VEGF pathway inhibitor or an EGF pathway inhibitor. In one embodiment said combination is used for the treatment of lung cancer, especially NSCLC.

In one embodiment the one or more chemotherapeutic agent to be combined with the IL-1β binding antibody or fragment thereof, suitably canakinumab or gevokizumab, is a check-point inhibitor. In one further embodiment, said check-point inhibitor is nivolumab or pembrolizumab. In one further embodiment, said check-point inhibitor is atezolizumab. In one further embodiment, said check-point inhibitor is PDR-001 (spartalizumab). In one embodiment, said check-point inhibitor is durvalumab. In one embodiment, said check-point inhibitor is avelumab. Immunotherapies that target immune checkpoints, also known as checkpoint inhibitors, are currently emerging as key agents in cancer therapy. The immune checkpoint inhibitor can be an inhibitor of the receptor or an inhibitor of the ligand. Examples of the inhibiting targets include but not limited to a co-inhibitory molecule (e.g., a PD-1 inhibitor (e.g., an anti-PD-1 antibody molecule), a PD-L1 inhibitor (e.g., an anti-PD-L1 antibody molecule), a PD-L2 inhibitor (e.g., an anti-PD-L2 antibody molecule), a LAG-3 inhibitor (e.g., an anti-LAG-3 antibody molecule), a TIM-3 inhibitor (e.g., an anti-TIM-3 antibody molecule)), an activator of a co-stimulatory molecule (e.g., a GITR agonist (e.g., an anti-GITR antibody molecule)), a cytokine (e.g., IL-15 complexed with a soluble form of IL-15 receptor alpha (IL-15Ra)), an inhibitor of cytotoxic T-lymphocyte-associated protein 4 (e.g., an anti-CTLA-4 antibody molecule) or any combination thereof.

### PD-1 Inhibitors

In one aspect of the invention, the IL-1β inhibitor or a functional fragment thereof is administered together with a PD-1 inhibitor. In one some embodiment the PD-1 inhibitor is chosen from PDR001(spartalizumab) (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

In one embodiment, the PD-1 inhibitor is an anti-PD-1 antibody. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

TABLE A
Amino acid and nucleotide sequences of exemplary anti-PD-1 antibody molecules

| | | |
|---|---|---|
| BAP049-Clone-B HC | VH | EVQLVQSGAEVKKPGESLRISCKGSGYTFTTYWMHWVRQAT |
| SEQ ID NO: 506 | | GQGLEWMGNIYPGTGGSNFDEKFKNRVTITADKSTSTAYMEL |
| | | SSLRSEDTAVYYCTRWTTGTGAYWGQGTTVTVSS |
| BAP049-Clone-B LC | VL | EIVLTQSPATLSLSPGERATLSCKSSQSLLDSGNQKNFLTWYQQ |
| SEQ ID NO: 516 | | KPGKAPKLLIYWASTRESGVPSRFSGSGSGTDFTFTISSLQPEDI |
| | | ATYYCQNDYSYPYTFGQGTKVEIK |
| BAP049-Clone-E HC | VH | EVQLVQSGAEVKKPGESLRISCKGSGYTFTTYWMHWVRQAT |
| SEQ ID NO: 506 | | GQGLEWMGNIYPGTGGSNFDEKFKNRVTITADKSTSTAYMEL |
| | | SSLRSEDTAVYYCTRWTTGTGAYWGQGTTVTVSS |
| BAP049-Clone-E LC | VL | EIVLTQSPATLSLSPGERATLSCKSSQSLLDSGNQKNFLTWYQQ |
| SEQ ID NO: 520 | | KPGQAPRLLIYWASTRESGVPSRFSGSGSGTDFTFTISSLEAEDA |
| | | ATYYCQNDYSYPYTFGQGTKVEIK |

In one embodiment, the anti-PD-1 antibody is spartalizumab.

In one embodiment, the anti-PD-1 antibody is Nivolumab.

In one embodiment, the anti-PD-1 antibody molecule is Pembrolizumab.

In one embodiment, the anti-PD-1 antibody molecule is Pidilizumab.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. Other exemplary anti-PD-1 molecules include REGN2810 (Regeneron), PF-06801591 (Pfizer), BGB-A317/BGB-108 (Beigene), INCSHR1210 (Incyte) and TSR-042 (Tesaro).

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

### PD-L1 Inhibitors

In one aspect of the invention, the IL-1β inhibitor or a functional fragment thereof is administered together with a PD-L1 inhibitor. In some embodiments, the PD-L1 inhibitor is chosen from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

TABLE B
Amino acid and nucleotide sequences of exemplary anti-PD-Ll antibody molecules

| | | |
|---|---|---|
| BAP058-Clone O HC | VH | EVQLVQSGAEVKKPGATVKISCKVSGYTFTSYWMYWVRQA |
| SEQ ID NO: 606 | | RGQRLEWIGRIDPNSGSTKYNEKFKNRFTISRDNSKNTLYLQ |
| | | MNSLRAEDTAVYYCARDYRKGLYAMDYWGQGTTVTVSS |
| BAP058-Clone O LC | VL | AIQLTQSPSSLSASVGDRVTITCKASQDVGTAVAWYLQKPGQ |
| SEQ ID NO: 616 | | SPQLLIYWASTRHTGVPSRFSGSGSGTDFTFTISSLEAEDAATY |
| | | YCQQYNSYPLTFGQGTKVEIK |
| BAP058-Clone N HC | VH | EVQLVQSGAEVKKPGATVKISCKVSGYTFTSYWMYWVRQA |
| SEQ ID NO: 620 | | TGQGLEWMGRIDPNSGSTKYNEKFKNRVTITADKSTSTAYME |
| | | LSSLRSEDTAVYYCARDYRKGLYAMDYWGQGTTVTVSS |
| BAP058-Clone N LC | VL | DVVMTQSPLSLPVTLGQPASISCKASQDVGTAVAWYQQKPG |
| SEQ ID NO: 624 | | QAPRLLIYWASTRHTGVPSRFSGSGSGTEFTLTISSLQPDDFAT |
| | | YYCQQYNSYPLTFGQGTKVEIK |

In one embodiment, the anti-PD-L1 antibody molecule is Atezolizumab (Genentech/Roche), also known as MPDL3280A, RG7446, R05541267, YW243.55.570, or TECENTRIQ™. Atezolizumab and other anti-PD-L1 antibodies are disclosed in U.S. Pat. No. 8,217,149, incorporated by reference in its entirety.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

### LAG-3 Inhibitors

In one aspect of the invention, the IL-1β inhibitor or a functional fragment thereof is administered together with a LAG-3 inhibitor. In some embodiments, the LAG-3 inhibitor is chosen from LAG525 (Novartis), BMS-986016 (Bristol-Myers Squibb), TSR-033 (Tesaro), IMP731 or GSK2831781 and IMP761 (Prima BioMed).

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

TABLE C
Amino acid and nucleotide sequences of exemplary anti-LAG-3 antibody molecules

| | | |
|---|---|---|
| BAP050-Clone I HC | VH | QVQLVQSGAEVKKPGASVKVSCKASGFTLTNYGMNWVRQAR |
| SEQ ID NO: 706 | | GQRLEWIGWINTDTGEPTYADDFKGRFVFSLDTSVSTAYLQISS |
| | | LKAEDTAVYYCARNPPYYYGTNNAEAMDYWGQGTTVTVSS |
| BAP050-Clone I LC | VL | DIQMTQSPSSLSASVGDRVTITCSSSQDISNYLNWYLQKPGQSP |
| SEQ ID NO: 718 | | QLLIYYTSTLHLGVPSRFSGSGSGTEFTLTISSLQPDDFATYYCQ |
| | | QYYNLPWTFGQGTKVEIK |
| BAP050-Clone J HC | VH | QVQLVQSGAEVKKPGASVKVSCKASGFTLTNYGMNWVRQAP |
| SEQ ID NO: 724 | | GQGLEWMGWINTDTGEPTYADDFKGRFVFSLDTSVSTAYLQI |
| | | SSLKAEDTAVYYCARNPPYYYGTNNAEAMDYWGQGTTVTVS |
| | | S |
| BAP050-Clone J LC | VL | DIQMTQSPSSLSASVGDRVTITCSSSQDISNYLNWYQQKPGKAP |
| SEQ ID NO: 730 | | KLLIYYTSTLHLGIPPRFSGSGYGTDFTLTINNIESEDAAYYFCQ |
| | | QYYNLPWTFGQGTKVEIK |

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table D.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table D.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE D
Amino acid sequences of exemplary anti-LAG-3 antibody molecules
BMS-986016

| | | |
|---|---|---|
| SEQ ID NO: 762 | Heavy chain | QVQLQQWGAGLLKPSETLSLTCAVYGGSFSDYYWNWIRQPPGKG |
| | | LEWIGEINHRGSTNSNPSLKSRVTLSLDTSKNQFSLKLRSVTAADTA |
| | | VYYCAFGYSDYEYNWFDPWGQGTLVTVSSASTKGPSVFPLAPCSR |
| | | STSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLGK |
| SEQ ID NO: 763 | Light chain | EIVLTQSPATLSLSPGERATLSCRASQSISSYLAWYQQKPGQAPRLL |
| | | IYDASNRATGIPARFSGSGSGTDFTLTISSLEPEDFAVYYCQQRSNW |
| | | PLTFGQGTNLEIKRTVAAPSVFIFPPSDEQLKSGTASVVCLLNNFYP |
| | | REAKVQWKVDNALQSGNSQESVTEQDSKDSTYSLSSTLTLSKADY |
| | | EKHKVYACEVTHQGLSSPVTKSFNRGEC |

IMP731

| | | |
|---|---|---|
| SEQ ID NO: 764 | Heavy chain | QVQLKESGPGLVAPSQSLSITCTVSGFSLTAYGVNWVRQPPGKGLE |
| | | WLGMIWDDGSTDYNSALKSRLSISKDNSKSQVFLKMNSLQTDDTA |
| | | RYYCAREGDVAFDYWGQGTTLTVSSASTKGPSVFPLAPSSKSTSG |
| | | GTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKE |
| | | YKCKVSNKALPAPIEKTISKAKGQPREPQVYTLPPSRDELTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPGK |
| SEQ ID NO: 765 | Light chain | DIVMTQSPSSLAVSVGQKVTMSCKSSQSLLNGSNQKNYLAWYQQ |
| | | KPGQSPKLLVYFASTRDSGVPDRFIGSGSGTDFTLTISSVQAEDLAD |
| | | YFCLQHFGTPPTFGGGTKLEIKRTVAAPSVFIFPPSDEQLKSGTASV |
| | | VCLLNNFYPREAKVQWKVDNALQSGNSQESVTEQDSKDSTYSLSS |
| | | TLTLSKADYEKHKVYACEVTHQGLSSPVTKSFNRGEC |

### TIM-3 Inhibitors

In one aspect of the invention, the IL-1β inhibitor or a functional fragment thereof is administered together with a TIM-3 inhibitor. In some embodiments, the TIM-3 inhibitor is MGB453 (Novartis) or TSR-022 (Tesaro).

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

The antibody molecules described herein can be made by vectors, host cells, and methods described in US 2015/0218274, incorporated by reference in its entirety.

TABLE E
Amino acid and nucleotide sequences of exemplary anti-TIM-3 antibody molecules
ABTIM3-hum11

| | | |
|---|---|---|
| SEQ ID NO: 806 | VH | QVQLVQSGAEVKKPGSSVKVSCKASGYTFTSYNMHWVRQA |
| | | PGQGLEWMGDIYPGNGDTSYNQKFKGRVTITADKSTSTVY |
| | | MELSSLRSEDTAVYYCARVGGAFPMDYWGQGTTVTVSS |
| SEQ ID NO: 816 | VL | AIQLTQSPSSLSASVGDRVTITCRASESVEYYGTSLMQWYQQ |
| | | KPGKAPKLLIYAASNVESGVPSRFSGSGSGTDFTLTISSLQPE |
| | | DFATYFCQQSRKDPSTFGGGTKVEIK |

ABTIM3-hum03

| | | |
|---|---|---|
| SEQ ID NO: 822 | VH | QVQLVQSGAEVKKPGASVKVSCKASGYTFTSYNMHWVRQ |
| | | APGQGLEWIGDIYPGQGDTSYNQKFKGRATMTADKSTSTVY |
| | | MELSSLRSEDTAVYYCARVGGAFPMDYWGQGTLVTVSS |
| SEQ ID NO: 826 | VL | DIVLTQSPDSLAVSLGERATINCRASESVEYYGTSLMQWYQ |
| | | QKPGQPPKLLIYAASNVESGVPDRFSGSGSGTDFTLTISSLQA |
| | | EDVAVYYCQQSRKDPSTFGGGTKVEIK |

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table F. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE F
Amino acid sequences of exemplary anti-TIM-3 antibody molecules
APE5137

| | | |
|---|---|---|
| SEQ ID NO: 830 | VH | EVQLLESGGGLVQPGGSLRLSCAAASGFTFSSYDMSWVRQAPGKGLDW |
| | | VSTISGGGTYTYYQDSVKGRFTISRDNSKNTLYLQMNSLRAEDTAVYYC |
| | | ASMDYWGQGTTVTVSSA |
| SEQ ID NO: 831 | VL | DIQMTQSPSSLSASVGDRVTITCRASQSIRRYLNWYHQKPGKAPKLLIYG |
| | | ASTLQSGVPSRFSGSGSGTDFTLTISSLQPEDFAVYYCQQSHSAPLTFGG |
| | | GTKVEIKR |

APE5121

| | | |
|---|---|---|
| SEQ ID NO: 832 | VH | EVQVLESGGGLVQPGGSLRLYCVASGFTFSGSYAMSWVRQAPGKGLE |
| | | WVSAISGSGGSTYYADSVKGRFTISRDNSKNTLYLQMNSLRAEDTAVY |
| | | YCAKKYYVGPADYWGQGTLVTVSSG |
| SEQ ID NO: 833 | VL | DIVMTQSPDSLAVSLGERATINCKSSQSVLYSSNNKNYLAWYQHKPGQP |
| | | PKLLIYWASTRESGVPDRFSGSGSGTDFTLTISSLQAEDVAVYYCQQYYS |
| | | SPLTFGGGTKIEVK |

### GITR Agonists

In one aspect of the invention, the IL-1β inhibitor or a functional fragment thereof is administered together with a GITR agonist. In some embodiments, the GITR agonist is GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises a VH comprising the amino acid sequence of SEQ ID NO: 901 and a VL comprising the amino acid sequence of SEQ ID NO: 902.

TABLE G
Amino acid and nucleotide sequences of exemplary anti-GITR antibody molecule

| | | |
|---|---|---|
| MAB7 | VH | EVQLVESGGGLVQSGGSLRLSCAASGFSLSSYGVDWVRQA |
| SEQ ID NO: 901 | | PGKGLEWVGVIWGGGGTYYASSLMGRFTISRDNSKNTLYL |
| | | QMNSLRAEDTAVYYCARHAYGHDGGFAMDYWGQGTLVT |
| | | VSS |
| SEQ ID NO: 902 | VL | EIVMTQSPATLSVSPGERATLSCRASESVSSNVAWYQQRPG |
| | | QAPRLLIYGASNRATGIPARFSGSGSGTDFTLTISRLEPEDFA |
| | | VYYCGQSYSYPFTFGQGTKLEIK |

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table H.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE H
Amino acid sequence of exemplary anti-GITR antibody molecules

| | | |
|---|---|---|
| BMS-986156 | VH | QVQLVESGGGVVQPGRSLRLSCAASGFTFSSYGMHWVRQAPGK |
| SEQ ID NO: 920 | | GLEWVAVIWYEGSNKYYADSVKGRFTISRDNSKNTLYLQMNSL |
| | | RAEDTAVYYCARGGSMVRGDYYYGMDVWGQGTTVTVSS |
| SEQ ID NO: 921 | VL | AIQLTQSPSSLSASVGDRVTITCRASQGISSALAWYQQKPGKAPK |
| | | LLIYDASSLESGVPSRFSGSGSGTDFTLTISSLQPEDFATYYCQQF |
| | | NSYPYTFGQGTKLEIK |

### IL15/IL-15Ra Complexes

In one aspect of the invention, the IL-1β inhibitor or a functional fragment thereof is administered together with an IL-15/IL-15Ra complex. In some embodiments, the IL-15/IL-15Ra complex is chosen from NIZ985 (Novartis), ATL-803 (Altor) or CYP0150 (Cytune).

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 1001 in Table I and the soluble form of human IL-15Ra comprises an amino acid sequence of SEQ ID NO:1002 in Table I, as described in WO 2014/066527, incorporated by reference in its entirety. The molecules described herein can be made by vectors, host cells, and methods described in WO 2007/084342, incorporated by reference in its entirety.

TABLE 1
Amino acid and nucleotide sequences of exemplary IL-15/IL-15Ra complexes

| | | |
|---|---|---|
| NIZ985 | Human IL-15 | NWVNVISDLKKIEDLIQSMHIDATLYTESDVHPSCKVTAMKCFLLE |
| SEQ ID NO: | | LQVISLESGDASIHDTVENLIILANNSLSSNGNVTESGCKECEELEEK |
| 1001 | | NIKEFLQSFVHIVQMFINTS |
| SEQ ID NO: | Human Soluble | ITCPPPMSVEHADIWVKSYSLYSRERYICNSGFKRKAGTSSLTECVL |
| 1002 | IL-15Ra | NKATNVAHWTTPSLKCIRDPALVHQRPAPPSTVTTAGVTPQPESLS |
| | | PSGKEPAASSPSSNNTAATTAAIVPGSQLMPSKSPSTGTTEISSHESS |
| | | HGTPSQTTAKNWELTASASHQPPGVYPQG |

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety. In one embodiment, the IL-15/IL-15Ra Fc fusion protein comprises the sequences as disclosed in Table J.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety. In one embodiment, the IL-15/IL-15Ra sushi domain fusion comprises the sequences as disclosed in Table J.

TABLE J
Amino acid sequences of other exemplary IL-15/IL-15Ra complexes
ALT-803 (Altor)

| | | |
|---|---|---|
| SEQ ID NO: | IL-15N72D | NWVNVISDLKKIEDLIQSMHIDATLYTESDVHPSCKVTA |
| 1003 | | MKCFLLELQVISLESGDASIHDTVENLIILANDSLSSNGN |
| | | VTESGCKECEELEEKNIKEFLQSFVHIVQMFINTS |
| SEQ ID NO: | IL-15RaSu/ Fc | ITCPPPMSVEHADIWVKSYSLYSRERYICNSGFKRKAGTSSLTECVL |
| 1004§ | | NKATNVAHWTTPSLKCIREPKSCDKTHTCPPCPAPELLGGPSVFLFP |
| | | PKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSV |
| | | MHEALHNHYTQKSLSLSPGK |

IL-15 / IL-15Ra sushi domain fusion (Cytune)

| | | |
|---|---|---|
| SEQ ID | Human IL-15 | NWVNVISDLKKIEDLIQSMHIDATLYTESDVHPSCKVTAMKCFLLE |
| NO: 1005 | | LQVISLESGDASIHDTVENLIILANNSLSSNGNVTESGCKECEELEXK |
| | | NIKEFLQSFVHIVQMFINTS |
| | | Where X is E or K |
| SEQ ID | Human IL- | ITCPPPMSVEHADIWVKSYSLYSRERYICNSGFKRKAGTSSLTECVL |
| NO: 1006 | 15Ra sushi and | NKATNVAHWTTPSLKCIRDPALVHQRPAPP |
| | hinge domains | |

### CTLA-4 Inhibitors

In one aspect of the invention, the IL-1β inhibitor or a functional fragment thereof is administered together with an inhibitor of CTLA-4. In some embodiments, the CTLA-4 inhibitor is an anti-CTLA-4 antibody or fragment thereof. Exemplary anti-CTLA-4 antibodies include Tremelimumab (formerly ticilimumab, CP-675,206); and Ipilimumab (MDX-010, Yervoy®).

In one embodiment, the present invention provides an IL-1β antibody or a functional fragment thereof for use in the treatment of lung cancer, especially NSCLC, wherein said IL-1β antibody or a functional fragment thereof is administered in combination with one or more chemotherapeutic agent, wherein said one or more chemotherapeutic agent is a check point inhibitor, preferably selected from the group consisting of nivolumab, pembrolizumab, atezolizumab, avelumab, durvalumab, PDR-001(spartalizumab) and Ipilimumab. In one embodiment the one or more chemotherapeutic agent is a PD-1 or PD-L-1 inhibitor, preferably selected from the group consisting of nivolumab, pembrolizumab, atezolizumab, avelumab, durvalumab, PDR-001(spartalizumab). Typically cancer having at least partial inflammatory basis includes but not limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer. In one further embodiment, the IL-1β antibody is canakinumab or a functional fragment thereof. In one further embodiment, the IL-1β antibody is canakinumab or a functional fragment thereof. In one embodiment canakinumab is administered at a dose of 300 mg monthly. In one embodiment canakinumab is administered at a dose of 200 mg every 3 weeks or monthly. In one embodiment canakinumab is administered subcutaneously. In one further embodiment, the IL-1β antibody is canakinumab or a functional fragment thereof is administered in combination with a PD-1 or PD-L1 inhibitor, prefereably selected from nivolumab, pembrolizumab, atezolizumab, avelumab, durvalumab and PDR-001(spartalizumab), particularly with atezolizumab, wherein canakinumab is administered at the same time of the PD-1 or PD-L1 inhibitor. In one further embodiment, the IL-1β antibody is gevokizumab or a functional fragment thereof. In one embodiment gevokizumab is administered at a dose of 90 mg to about 360 mg, 90 mg to about 270 mg, 120 mg to 270 mg, 90 mg to 180 mg, 120 mg to 180 mg, 120 mg or 90 mg or 60 mg to 90 mg every 3 weeks. In one embodiment gevokizumab or a functional fragment thereof is administered at a dose of 120 mg every 3 weeks. In one embodiment, gevokizumab is administered every month at a dose of 90 mg to about 360 mg, 90 mg to about 270 mg, 120 mg to 270 mg, 90 mg to 180 mg, 120 mg to 180 mg, 120 mg or 90 mg or 60 mg to 90 mg. In one embodiment gevokizumab or a functional fragment thereof is administered at a dose of 120 mg every 4 weeks (monthly). In one embodiment gevokizumab is administered subcutaneously or preferably intravenously.

In one further embodiment, the IL-1β antibody is gevokizumab or a functional fragment thereof is administered in combination with a PD-1 or PD-L1 inhibitor, prefereably selected from nivolumab, pembrolizumab, atezolizumab, avelumab, durvalumab and PDR-001/spartalizumab, particularly with atezolizumab, wherein gevokizumab is administered at the same time as the PD-1 or PD-L1 inhibitor.

In one embodiment said patient has a tumor that has high PD-L1 expression [Tumor Proportion Score (TPS)≥50%)] as determined by an FDA-approved test, with or without EGFR or ALK genomic tumor aberrations. In one embodiment said patient has tumor that has PD-L1 expression (TPS≥1%) as determined by an FDA-approved test.

The term “in combination with” is understood as the two or more drugs are administered subsequently or simultaneously. Alternatively, the term “in combination with” is understood that two or more drugs are administered in the manner that the effective therapeutical concentration of the drugs are expected to be overlapping for a majority of the period of time within the patient's body. The DRUG of the invention and one or more combination partner (e.g. another drug, also referred to as “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The drug administered to a patient as separate entities either simultaneously, concurrently or sequentially with no specific time limits, wherein such administration provides therapeutically effective levels of the two compounds in the body of the patient and the treatment regimen will provide beneficial effects of the drug combination in treating the conditions or disorders described herein. The latter also applies to cocktail therapy, e.g. the administration of three or more active ingredients.

In one embodiment, the present invention provides an IL-1β antibody or a functional fragment thereof, suitably canakinumab or a functional fragment thereof or gevokizumab or a functional fragment thereof, for use in the treatment of lung cancer, wherein the lung cancer is an advanced, metastatic, relapsed, and/or refractory lung cancer. In one embodiment, the lung cancer is metastatic NSCLC.

In one embodiment, the present invention provides an IL-1β antibody or a functional fragment thereof, suitably canakinumab or a functional fragment thereof or gevokizumab or a functional fragment thereof, for use as the first line treatment of cancer having at least a partial inflammatory basis. Typically cancer having at least partial inflammatory basis includes but is not limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer. In one embodiment, the present invention provides an IL-1β antibody or a functional fragment thereof, suitably canakinumab or a functional fragment thereof or gevokizumab or a functional fragment thereof, for use as the first line treatment of cancer having at least a partial inflammatory basis, including lung cancer, especially NSCLC, especially for patients with expression or overexpression of IL-1β or IL-1 receptor. The term “first line treatment” means said patient is given the IL-1β antibody or a functional fragment thereof before the patient develops resistance to one or more other chemotherapeutic agent. Preferably one or more other chemotherapeutic agent is a platinum-based mono or combination therapy, a targeted therapy, such a tyrosine inhibitor therapy, a checkpoint inhibitor therapy or any combination thereof. As first line treatment, the IL-1β antibody or a functional fragment thereof, such as canakinumab or gevokizumab, can be administered to patient as monotherapy or preferably in combination with an check point inhibitor, particularly a PD-1 or PD-L1 inhibitor, particularly atezolizumab, with or without one or more small molecule chemotherapeutic agent.

In one preferred embodiment, canakinumab or a fragment thereof is used as the first line treatment of lung cancer, especially NSCLC, in combination with one check point inhibitor. As first line treatment, the IL-1β antibody or a functional fragment thereof can be administered to patient as monotherapy or preferably in combination with standard of care, such as one or more chemotherapeutic agent, especially with FDA-approved therapy for lung cancer, especially for NSCLC. In one preferred embodiment, canakinumab or a fragment thereof is used as the first line treatment of lung cancer, especially NSCLC, in combination with one check point inhibitor, preferably with a checkpoint inhibitor selected from nivolumab, pembrolizumab and PDR-001/spartalizumab avelumab, durvalumab and atezolizumab, preferably atezolizumab. In one preferred embodiment, said checkpoint inhibitor is pembrolizumab. In one preferred embodiment, said checkpoint inhibitor is spartalizumab. In one further preferred embodiment, at least one more chemotherapeutic agent is added on top of the combination above, preferably a platinum agent, such as cisplatin or a mitotic inhibitor, such as docetaxel. In one embodiment, canakinumab is administered at a dose of 200 mg every 3 weeks, preferably subcutaneously, subsequently or preferably simultaneously with the checkpoint inhibitor.

In one preferred embodiment, gevokizumab or a fragment thereof is used as the first line treatment of lung cancer, especially NSCLC, in combination with one check-point inhibitor, preferably with a PD-1/PD-L1 inhibitor selected from nivolumab, pembrolizumab and PDR-001/spartalizumab, avelumab, durvalumab and atezolizumab, preferably atezolizumab. In one preferred embodiment, said checkpoint inhibitor is pembrolizumab. In one preferred embodiment, said checkpoint inhibitor is spartalizumab. In one further preferred embodiment, at least one more chemotherapeutic agent is added on top of the combination above, preferably a platinum agent, such as cisplatin or a mitotic inhibitor, such as docetaxel. In one embodiment, gevokizumab is administered at a dose of 60 mg to 90 mg every 3 weeks or at a dose of 120 mg every 3 or 4 weeks or at a dose of 90 mg every 3 or 4 weeks, preferably intravenously, subsequently or preferably simultaneously with the checkpoint inhibitor.

In one embodiment, the present invention provides an IL-1β antibody or a functional fragment thereof, suitably canakinumab or a functional fragment thereof or gevokizumab or a functional fragment thereof, for use as the second or third line treatment of cancer having at least a partial inflammatory basis, including lung cancer, especially NSCLC. The term “the second or third line treatment” means IL-1β antibody or a functional fragment thereof is administered to a patient with cancer progression on or after one or more other chemotherapeutic agent treatment, especially disease progression on or after FDA-approved therapy for lung cancer, especially for NSCLC. Preferably one or more other chemotherapeutic agent is a platinum-based mono or combination therapy, a targeted therapy, such a tyrosine inhibitor therapy, a checkpoint inhibitor therapy or any combination thereof. As the second or third line treatment, the IL-1β antibody or a functional fragment thereof can be administered to the patient as monotherapy or preferably in combination with one or more chemotherapeutic agent, including the continuation of the early treatment with the same one or more chemotherapeutic agent.

For use as the second or third line treatment, the IL-1β antibody or a functional fragment thereof, such as canakinumab or gevokizumab, can be administered to patient as monotherapy or preferably in combination with a check-point inhibitor, particularly a PD-1 or PD-L1 inhbitor, particularly atezolizumab, with or without one or more small molecule chemotherapeutic agent.

In one preferred embodiment, canakinumab or a fragment thereof is used as second or third line treatment of lung cancer, especially NSCLC, in combination with one check point inhibitor, preferably with a checkpoint inhibitor selected from nivolumab, pembrolizumab and PDR-001/spartalizumab (Novartis), ipilimumaband atezolizumab, preferably atezolizumab. In one preferred embodiment, said checkpoint inhibitor is pembrolizumab. In one preferred embodiment, said checkpoint inhibitor is spartalizumab. In one further preferred embodiment, at least one more chemotherapeutic agent is added on top of the combination above, preferably a platinum agent, such as cisplatin or a mitotic inhibitor, such as docetaxel. In one embodiment, canakinumab is administered at a dose of 200 mg every 3 weeks, preferably subcutaneously, subsequently or preferably simultaneously with the checkpoint inhibitor.

In one preferred embodiment, gevokizumab or a fragment thereof is used as second or third line treatment of lung cancer, especially NSCLC or colorectal cancer, in combination with one check-point inhibitor, preferably with a PD-1/PD-L1 inhibitor selected from nivolumab, pembrolizumab and PDR-001/spartalizumab (Novartis) and atezolizumab, preferably atezolizumab. In one further preferred embodiment, at least one more chemotherapeutic agent is added on top of the combination above, preferably a platinum agent, such as cisplatin or a mitotic inhibitor, such as docetaxel. In one embodiment, gevokizumab is administered at a dose of 60 mg to 90 mg every 3 weeks or at a dose of 120 mg every 3 or 4 weeks, preferably intravenously, subsequently or preferably simultaneously with the checkpoint inhibitor.

In one embodiment, the present invention provides an IL-1β antibody or a functional fragment thereof for use in the treatment of lung cancer in a subject as adjuvant therapy following standard of care for each stage, wherein patient has high risk NSCLC (Stage IB, 2 or 3A), wherein the lung cancer has been surgically removed (surgical resection). In one embodiment, said adjuvant treatment will last for at least 6 months, preferably at least one year, preferably one year. In one embodiment, said IL-1β antibody or a functional fragment thereof is gevokizumab. In one embodiment, said IL-1β antibody or a functional fragment thereof is canakinumab. In one embodiment, canakinumab is administered at a dose of 300 mg monthly, preferably for at least one year. In one embodiment, canakinumab is administered at a dose of 200 mg every 3 weeks or monthly, preferably subcutaneously, preferably for at least one year.

In one embodiment, the present invention provides canakinumab or a functional fragment thereof for use in the treatment of lung cancer in a subject as adjuvant therapy following surgical removal of the lung cancer. Preferably, said patient has completed standard chemotherapy treatment, for example 4 cycles of cisplatin based chemotherapy. In one embodiment, canakinumab is administered monthly at a dose of 200 mg, preferably for at least one year. In one embodiment, canakinumab is administered at a dose of 200 mg every 3 weeks or monthly, preferably subcutaneously, preferably for at least one year. In one embodiment the present invention provides an IL-1β antibody or a functional fragment thereof for use as the first line treatment of NSCLC in a patient, wherein said patient has Stage 3B (not amenable to chemo/radiation) or stage 4 disease, alone or preferably in combination with standard of care. In one embodiment, said IL-1β antibody or a functional fragment thereof is gevokizumab. In one embodiment, said IL-1β antibody or a functional fragment thereof is canakinumab. In one embodiment, canakinumab is administered monthly at a dose of at least 300 mg, preferably monthly at a dose of 300 mg. In one embodiment, canakinumab is administered at a dose of 200 mg every 3 weeks or monthly, preferably subcutaneously. In one embodiment the present invention provides an IL-1β antibody or a functional fragment thereof for use in the treatment of NSCLC in patients, wherein said patient has disease progression on or after the treatment with one or more checkpoint inhibitors, preferably a PD-1/PD-L1 inhibitor, preferably atezolizumab. In one embodiment, said patient has disease progression after treatment with one or more chemotherapeutic agent other than one or more checkpoint inhibitors, preferably a PD-1 inhibitor, preferably atezolizumab. In one embodiment said PD-1 inhibitor is selected from nivolumab, pembrolizumab, atezolizumab, avelumab, durvalumaband PDR-001(spartalizumab). In one embodiment, said IL-1β antibody or a functional fragment thereof is gevokizumab. In one embodiment, said IL-1β antibody or a functional fragment thereof is canakinumab. In one embodiment, canakinumab is administered monthly at a dose of at least 300 mg, preferably monthly at a dose of 300 mg. In one embodiment, canakinumab is administered at a dose of from 200 mg to 300 mg per treatment, wherein canakinumab is administered preferably every 3 weeks or preferably monthly. In one embodiment, canakinumab is administered at a dose of 200 mg every 3 weeks. The IL-1β antibody or a functional fragment thereof, particularly canakinumab or gevokizumab, is administered as monotherapy or preferably in combination with one or more chemotherapeutic agent, including the continuation of the earlier treatment with the same one or more chemotherapeutic agent.

In one embodiment the present invention provides an IL-1β antibody or a functional fragment thereof for use in the treatment of colorectal cancer (CRC) or gastric-intestinal cancer in a patient as monotherapy or preferably in combination with standard of care. In one embodiment, said IL-1β antibody or a functional fragment thereof is gevokizumab. In one embodiment gevokizumab is administered at a dose of from 60 mg to 90 mg per treatment, wherein gevokizumab is administered preferably every 3 weeks or preferably monthly. In one embodiment gevokizumab is administered at a dose of 120 mg per treatment, wherein gevokizumab is administered preferably every 3 weeks or preferably monthly. In one embodiment, said IL-1β antibody or a functional fragment thereof is canakinumab. In one embodiment, canakinumab is administered monthly at a dose of at least 300 mg, preferably monthly at a dose of 300 mg. In one embodiment, canakinumab is administered at a dose of from 200 mg to 300 mg per treatment, wherein canakinumab is administered preferably every 3 weeks or preferably monthly. In one embodiment, canakinumab is administered 200 mg every 3 weeks.

In a preferred embodiment the anti-PD-1 antibody molecule is PDR001/spartalizumab.

In a preferred embodiment the anti-PD-1 antibody molecule is pembrolizumab.

In a preferred embodiment the anti-PD-1 antibody molecule is atezolizumab.

In a preferred embodiment the anti-PD-1 antibody molecule is nivolumab.

In certain embodiments, the present invention provides an IL-1β antibody or a functional fragment thereof, suitably gevokizumab or a functional fragment thereof, suitably canakinumab or a functional fragment thereof, for use in the treatment of renal cell carcinoma (RCC). The term “renal cell carcinoma (RCC)” as used herein refers to a cancer of the kidney arising from the epithelium of the renal tubules within the renal cortex and includes primary renal cell carcinoma, locally advanced renal cell carcinoma, unresectable renal cell carcinoma, metastatic renal cell carcinoma, refractory renal cell carcinoma, and/or cancer drug resistant renal cell carcinoma.

All the disclosed uses disclosed throughout this application, including but not limited to, doses and dosing regimens, combinations, route of administration and biomarkers can be applied to the treatment of renal cell carcinoma. In one embodiment, canakinumab is administered at a dose of from 200 mg to 400 mg per treatment, wherein canakinumab is administered preferably every 3 weeks or preferably monthly. In one embodiment, canakinumab is administered at a dose of 200 mg every 3 weeks, preferably subcutaneously. In one embodiment, gevokizumab is administered at a dose of from 90 mg to 200 mg per treatment, wherein gevokizumab is administered preferably every 3 weeks or preferably monthly. In one embodiment, gevokizumab is administered at a dose of 120 mg every 3 weeks or monthly, preferably intravenously.

In one embodiment, the present invention provides gevokizumab or a functional fragment thereof, for use in the treatment of renal cell carcinoma (RCC), wherein gevokizumab, or a functional fragment thereof, is administered in combination with one or more chemotherapeutic agent. In one embodiment the chemotherapeutic agent is the standard of care agent for renal cell carcinoma (RCC). In one embodiment the one or more chemotherapeutic agent is selected from everolimus (Afinitor®), aldesleukin (Proleukin®), bevacizumab (Avastin®), axitinib (Inlyta®), cabozantinib (Cabometyx®), lenvatinib mesylate (Lenvima®), sorafenib tosylate (Nexavar®), nivolumab (Opdivo®), pazopanib hydrochloride (Votrient®), sunitinib malate (Sutent®), temsirolimus (Torisel®), ipilimumab and tivozanib (FOTIVDA®). Depending on the patient condition, at least one, at least two or at least three chemotherapeutic agents can be selected from the list above, to be combined with gevokizumab.

In one embodiment the one or more chemotherapeutic agent is a CTLA-4 checkpoint inhibitor, wherein preferably said CTLA-4 checkpoint inhibitor is ipilimumab. In one embodiment the one or more chemotherapeutic agent is everolimus.

In one embodiment the one or more chemotherapeutic agent is a checkpoint inhibitor, wherein preferably is a PD-1 or PD-L1 inhibitor, wherein preferably selected from the group consisting of nivolumab, pembrolizumab, atezolizumab, avelumab, durvalumab and spartalizumab (PDR-001).

In one embodiment the one or more chemotherapeutic agent is nivolumab. In one embodiment the one or more chemotherapeutic agent are nivolumab plus ipilimumab.

In one embodiment the or more chemotherapeutic agent is cabozantinib.

In one embodiment the or more chemotherapeutic agent is Atezolizumab plus bevacizumab.

In one embodiment, gevokizumab or a functional fragment thereof is used, alone or preferably in combination, in the prevention of recurrence or relapse of renal cell carcinoma (RCC) in a patient after said cancer has been surgically removed. In one embodiment, gevokizumab or a functional fragment thereof is used, alone or preferably in combination, in first line treatment of renal cell carcinoma (RCC). In one embodiment gevokizumab or a functional fragment thereof is used, alone or preferably in combination, in second or third line of renal cell carcinoma (RCC).

The above disclosed embodiments for gevokizumab or a functional fragment thereof are suitably applicable for canakinumab or a functional fragment thereof.

In certain embodiments, the present invention provides an IL-1β antibody or a functional fragment thereof, suitably gevokizumab or a functional fragment thereof, suitably canakinumab or a functional fragment thereof, for use in the treatment of colorectal cancer (CRC). The term “Colorectal cancer (CRC)”, also known as bowel cancer and colon cancer, as used herein means a neoplasm arising from the colon and/or rectum, particularly from the epithelium of the colon and/or rectum and includes colon adenocarcinoma, rectal adenocarcinoma, metastatic colorectal cancer (mCRC), advanced colorectal cancer, refractory colorectal cancer, refractory metastatic microsatellite stable (MSS) colorectal cancer unresectable colorectal cancer, and/or cancer drug resistant colorectal cancer. Up to 25% of patients are diagnosed with metastatic disease at presentation and 50% of patients may go on to develop metastases at some point in life.

All the disclosed uses disclosed throughout this application, including but not limited to, doses and dosing regimens, combinations, route of administration and biomarkers can be applied to the treatment of CRC. In one embodiment, canakinumab is administered at a dose of from 200 mg to 400 mg per treatment, wherein canakinumab is administered preferably every 3 weeks or preferably monthly. In one embodiment, canakinumab is administered at a dose of 200 mg every 3 weeks, preferably subcutaneously. In one embodiment, gevokizumab is administered at a dose of from 90 mg to 200 mg per treatment, wherein gevokizumab is administered preferably every 3 weeks or preferably monthly. In one embodiment, gevokizumab is administered at a dose of 120 mg every 3 weeks or monthly, preferably intravenously.

In one embodiment, the present invention provides gevokizumab or a functional fragment thereof, for use in the treatment of colorectal cancer (CRC), wherein gevokizumab, or a functional fragment thereof, is administered in combination with one or more chemotherapeutic agent. In one embodiment the chemotherapeutic agent is the standard of care agent for CRC. In one embodiment the one or more chemotherapeutic agent is selected from irinotecan hydrochloride (Camptosar®), capecitabine (Xeloda®), oxaliplatin (Eloxatin®), 5-FU (fluorouracil), leucovorin calcium (folinic acid), FU-LV/FL (5-FU plus leucovorin), trifluridine/tipiracil hydrochloride (Lonsurf®), nivolumab (Opdivo®), regorafenib (Stivarga®), FOLFOXIRI (leucovorin, 5-fluorouracil [5-FU], oxaliplatin, irinotecan), FOLFOX (leucovorin, 5-FU, oxaliplatin), FOLFIRI (leucovorin, 5-FU, irinotecan), CapeOx (capecitabine plus oxaliplatin), XELIRI (capecitabine (Xeloda®) plus irinotecan hydrochloride), XELOX (capecitabine (Xeloda®) plus oxaliplatin), FOLFOX plus bevacizumab (Avastin®), cetuximab (Erbitux®), panitumumab (Vectibix®), FOLFIRI plus Ramucirumab (Cyramza®), FOLFIRI plus cetuximab (Erbitux®), and FOLFIRI plus Ziv-aflibercept (Zaltrap). Depending on the patient condition, at least one, at least two or at least three chemotherapeutic agents can be selected from the list above, to be combined with gevokizumab.

In one embodiment the one or more chemotherapeutic agent is a general cytotoxic agent, wherein preferably said general cytotoxic agent is selected from the list consisting of FOLFOX, FOLFIRI, capecitabine, 5-fluorouracil, irinotecan and oxaliplatin.

Usually, the initial therapy of CRC involves a cytotoxic backbone of a doublet chemotherapy regimen, combining fluorouracil and oxaliplatin (FOLFOX), fluorouracil and irinotecan (FOLFIRI), or capecitabine and oxaliplatin (XELOX). Bevacizumab is typically recommended upfront combined with chemotherapy. For patients with wild-type RAS tumors anti-EGFR agents (cetuximab and/orpanitumumab) represent alternative options for initial biologic therapy in combination with backbone chemotherapy.

The term “FOLFOX” as used herein refers to a combination therapy (e.g., chemotherapy) comprising at least one oxaliplatin compound chosen from oxaliplatin, pharmaceutically acceptable salts thereof, and solvates of any of the foregoing; at least one 5-fluorouracil (also known as 5-FU) compound chosen from 5-fluorouracil, pharmaceutically acceptable salts thereof, and solvates of any of the foregoing; and at least one folinic acid compound chosen from folinic acid (also known as leucovorin), levofolinate (the levo isoform of folinic acid), pharmaceutically acceptable salts of any of the foregoing, and solvates of any of the foregoing. The term “FOLFOX” as used herein is not intended to be limited to any particular amounts of or dosing regimens for those components.

The term “FOLFIRI” as used herein refers to a combination therapy (e.g., chemotherapy) comprising at least one irinotecan compound chosen from irinotecan, pharmaceutically acceptable salts thereof, and solvates of any of the foregoing; at least one 5-fluorouracil (also known as 5-FU) compound chosen from 5-fluorouracil, pharmaceutically acceptable salts thereof, and solvates of any of the foregoing; and at least one compound chosen from folinic acid (also known as leucovorin), levofolinate (the levo isoform of folinic acid), pharmaceutically acceptable salts of any of the foregoing, and solvates of any of the foregoing. The term “FOLFIRI” as used herein is not intended to be limited to any particular amounts of or dosing regimens for these components. Rather, as used herein, “FOLFIRI” includes all combinations of these components in any amounts and dosing regimens.

In one embodiment the one or more chemotherapeutic agent is a VEGF inhibitor (e.g., an inhibitor of one or more of VEGFR (e.g., VEGFR-1, VEGFR-2, or VEGFR-3) or VEGF).

Exemplary VEGFR pathway inhibitors that can be used in combination with an IL-1β binding antibody or a functional fragment thereof, suitably gevokizumab, for use in the treatment of cancer with partial inflammatory basis, include, e.g., bevacizumab (also known as rhuMAb VEGF or AVASTIN®), ramucirumab (Cyramza®), ziv-aflibercept (Zaltrap®), cediranib (RECENTIN™, AZD2171), lenvatinib (Lenvima®), vatalanib succinate, axitinib (INLYTA®); brivanib alaninate (BMS-582664, (S)—((R)-1-(4-(4-Fluoro-2-methyl-1H-indol-5-yloxy)-5-methylpyrrolo[2, 1-f][1,2,4]triazin-6-yloxy)propan-2-yl)2-aminopropanoate); sorafenib (NEXAVAR®); pazopanib (VOTRIENT®); sunitinib malate (SUTENT®); cediranib (AZD2171, CAS 288383-20-1); vargatef (BIBF1120, CAS 928326-83-4); Foretinib (GSK1363089); telatinib (BAY57-9352, CAS 332012-40-5); apatinib (YN968D1, CAS 811803-05-1); imatinib (GLEEVEC®); ponatinib (AP24534, CAS 943319-70-8); tivozanib (AV951, CAS 475108-18-0); regorafenib (BAY73-4506, CAS 755037-03-7); brivanib (BMS-540215, CAS 649735-46-6); vandetanib (CAPRELSA® or AZD6474); motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); semaxanib (SU5416), linfanib (ABT869, CAS 796967-16-3); cabozantinib (XL184, CAS 849217-68-1); lestaurtinib (CAS 111358-88-4); N-[5-[[[5-(1,1-dimethylethyl)-2-oxazolyl]methyl]thio]-2-thiazolyl]-4-piperidinecarboxamide (BMS38703, CAS 345627-80-7); (3R,4R)-4-amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and endostatin (ENDOSTAR®).

In one embodiment the one or more chemotherapeutic agent is anti-VEGF antibody. In one embodiment the one or more chemotherapeutic agent is anti-VEGF inhibitor of small molecule weight.

In one embodiment the one or more chemotherapeutic agent is a VEGF inhibitor is selected from the list consisting of bevacizumab, ramucirumab and ziv-aflibercept. In one preferred embodiment the VEGF inhibitor is bevacizumab.

In one embodiment the one or more chemotherapeutic agent is FOLFIRI plus bevacizumab or FOLFOX plus bevacizumab.

In one embodiment the one or more chemotherapeutic agent is a checkpoint inhibitor, preferably a PD-1 or PD-L1 inhibitor, preferably selected from the group consisting of nivolumab, pembrolizumab, atezolizumab, avelumab, durvalumab and spartalizumab (PDR-001). In one preferred embodiment embodiment the one or more chemotherapeutic agent is pembrolizumab. In one preferred embodiment embodiment the one or more chemotherapeutic agent is nivolumab.

In one preferred embodiment embodiment the one or more chemotherapeutic agent is atezolizumab. In one further preferred embodiument the one or more chemotherapeutic agent is atezolizumab and cobimetinib.

In one preferred embodiment embodiment the one or more chemotherapeutic agent is ramucirumab. In one preferred embodiment said patient has metastatic CRC.

In one preferred embodiment embodiment the one or more chemotherapeutic agent is ziv-aflibercept. In one preferred embodiment said patient has metastatic CRC.

In one preferred embodiment embodiment the one or more chemotherapeutic agent is a a tyrosine kinase inhibitor. In one embodiment said tyrosine kinase inhibitor is an EGF pathway inhibitor, prefearbyl an inhibitor of Epidermal Growth Factor Receptor (EGFR). Preferably the EGFR inhibitor is chosen from one of more of erlotinib (Tarceva®), gefitinib (Iressa®), cetuximab (Erbitux®), panitumumab (Vectibix®), necitumumab (Portrazza®), dacomitinib, nimotuzumab, imgatuzumab, osimertinib (Tagrisso®), lapatinib (TYKERB®, TYVERB®). In one embodiment said EGFR inhibitor is cetuximab. n one embodiment said EGFR inhibitor is panitumumab.

In one embodiment, the EGFR inhibitor is (R,E)-N-(7-chloro-1-(1-(4-(dimethylamino)but-2-enoyl)azepan-3-yl)-1H-benzo[d]imidazol-2-yl)-2-methylisonicotinamide (Compound A40) or a compound disclosed in PCT Publication No. WO 2013/184757.

In one embodiment, gevokizumab or a functional fragment thereof is used, alone or preferably in combination, in the prevention of recurrence or relapse of CRC in a patient after said cancer has been surgically removed. In one embodiment, gevokizumab or a functional fragment thereof is used, alone or preferably in combination, in first line treatment of CRC. In one embodiment gevokizumab or a functional fragment thereof is used, alone or preferably in combination, in second or third line of CRC.

The above disclosed embodiments for gevokizumab or a functional fragment thereof are suitably applicable for canakinumab or a functional fragment thereof.

In certain embodiments, the present invention provides an IL-1β antibody or a functional fragment thereof, suitably gevokizumab or a functional fragment thereof, suitably canakinumab or a functional fragment thereof, for use in the treatment of gastric cancer.

As used herein, the term “gastric cancer” encompasses gastric and intestinal cancer and cancer of the esophagus (gastroesophageal cancer), particularly the lower part of the esophagus and refers to primary gastric cancer, metastatic gastric cancer, refractory gastric cancer, unresectable gastric cancer, and/or cancer drug resistant gastric cancer. The term “gastric cancer” includes adenocarcinoma of the distal esophagus, gastroesophageal junction and/or stomach, gastrointestinal carcinoid tumor, and gastrointestinal stromal tumor. In a preferred embodiment, the gastric cancer is gastroesophageal cancer.

All the disclosed uses throughout this application, including but not limited to, doses and dosing regimens, combinations, route of administration and biomarkers can be applied to the treatment of gastric cancer. In one embodiment, canakinumab is administered at a dose of from 200 mg to 400 mg per treatment, wherein canakinumab is administered preferably every 3 weeks or preferably monthly. In one embodiment, canakinumab is administered at a dose of 200 mg every 3 weeks, preferably subcutaneously. In one embodiment, gevokizumab is administered at a dose of from 90 mg to 200 mg per treatment, wherein gevokizumab is administered preferably every 3 weeks or preferably monthly. In one embodiment, gevokizumab is administered at a dose of 120 mg every 3 weeks or monthly, preferably intravenously.

In one embodiment, the present invention provides gevokizumab or a functional fragment thereof, for use in the treatment of gastric cancer, wherein gevokizumab, or a functional fragment thereof, is administered in combination with one or more chemotherapeutic agent. In one embodiment the chemotherapeutic agent is the standard of care agent for gastric cancer. In one embodiment the one or more chemotherapeutic agent is selected from carboplatin plus paclitaxel (Taxol®), cisplatin plus 5-fluorouracil (5-FU), ECF (epirubicin (Ellence®), cisplatin, and 5-FU), DCF (docetaxel (Taxotere®), cisplatin, and 5-FU), cisplatin plus capecitabine (Xeloda®), oxaliplatin plus 5-FU, oxaliplatin plus capecitabine, irinotecan (Camptosar®) ramucirumab (Cyramza®), docetaxel (Taxotere®), trastuzumab (Herceptin®), FU-LV/FL (5-fluorouracil plus leucovorin), and XELIRI (capecitabine (Xeloda®) plus irinotecan hydrochloride). Depending on the patient condition, at least one, at least two or at least three chemotherapeutic agents can be selected from the list above, to be combined with gevokizumab.

In one embodiment the one or ore chemotherapeutic agent is paclitaxel and ramucirumab. In one further embodiment said combination is used for second line treatment of metastatic gastroesophageal cancer.

In one embodiment the one or more chemotherapeutic agent is a checkpoint inhibitor, wherein preferably is a PD-1 or PD-L1 inhibitor, wherein preferably selected from the group consisting of nivolumab, pembrolizumab, atezolizumab, avelumab, durvalumab and spartalizumab (PDR-001).

In one embodiment the one or more chemotherapeutic agent is nivolumab. In one embodiment the one or more chemotherapeutic agent is nivolumab plus and ipilimumab. In one further embodiment said combination is used for first or second line treatment of metastatic gastroesophageal cancer.

In one embodiment, gevokizumab or a functional fragment thereof is used, alone or preferably in combination, in the prevention of recurrence or relapse of gastric cancer in a patient after said cancer has been surgically removed. In one embodiment, gevokizumab or a functional fragment thereof is used, alone or preferably in combination, in first line treatment of gastric cancer. In one embodiment gevokizumab or a functional fragment thereof is used, alone or preferably in combination, in second or third line of gastric cancer.

The above disclosed embodiments for gevokizumab or a functional fragment thereof are suitably applicable for canakinumab or a functional fragment thereof.

In certain embodiments, the present invention provides an IL-1β antibody or a functional fragment thereof, suitably gevokizumab or a functional fragment thereof, suitably canakinumab or a functional fragment thereof, for use in the treatment of melanoma. The term “melanoma” includes “malignant melanoma” and “cutaneous melanoma” and as used herein refers to a malignant tumor arising from melanocyte which are derived from the neural crest. Although most melanomas arise in the skin, they may also arise from mucosal surfaces or at other sites to which neural crest cells migrate. As used herein, the term “melanoma” includes primary melanoma, locally advanced melanoma, unresectable melanoma, BRAF V600 mutated melanoma, NRAS-mutant melanoma, metastatic melanoma (including unresectable or metastatic BRAF V600 mutated melanoma), refractory melanoma (including relapsed or refractory BRAF V600-mutant melanoma (e.g. said melanoma being relapsed after failure of BRAFi/MEKi combination therapy or refractory to BRAFi/MEKi combination therapy), cancer drug resistant melanoma (including BRAF-mutant melanoma resistant to BRAFi/MEKi combination treatment) and/or immuno-oncolocy (TO) refractory melanoma.

All the disclosed uses throughout this application, including but not limited to, doses and dosing regimens, combinations, route of administration and biomarkers can be applied to the treatment of melanoma. In one embodiment, canakinumab is administered at a dose of from 200 mg to 400 mg per treatment, wherein canakinumab is administered preferably every 3 weeks or preferably monthly, preferably subcutaneously. In one embodiment, canakinumab is administered at a dose of 200 mg every 3 weeks. In one embodiment, gevokizumab is administered at a dose of from 90 mg to 200 mg per treatment, wherein gevokizumab is administered preferably every 3 weeks or preferably monthly, preferably intravenously. In one embodiment, gevokizumab is administered at a dose of 90 mg every 3 weeks or monthly. In one embodiment, gevokizumab is administered at a dose of 120 mg every 3 weeks or monthly.

In one embodiment, the present invention provides gevokizumab or a functional fragment thereof, for use in the treatment of melanoma, wherein gevokizumab, or a functional fragment thereof, is administered in combination with one or more chemotherapeutic agent. In one embodiment the chemotherapeutic agent is the standard of care agent for melanoma. In one embodiment the one or more chemotherapeutic agent is selected from temozolomide, nab-paclitaxel, paclitaxel, cisplatin, carboplatin, vinblastine, aldesleukin (Proleukin®), cobimetinib (Cotellic®), Dacarbazine, Talimogene Laherparepvec (Imlygic®), (peg)interferon alfa-2b (Intron A®/Sylatron™), Trametinib (Mekinist®), Dabrafenib (Tafinlar®), Trametinib (Mekinist®) plus Dabrafenib (Tafinlar®), pembrolizumab (Keytruda®), Nivolumab (Opdivo®), Ipilimumab (Yervoy®), Nivolumab (Opdivo®) plus Ipilimumab (Yervoy®), and Vemurafenib (Zelboraf®). Other medicaments currently being development for the treatment of melanoma include atezolizumab (Tecentriq®) and atezolizumab (Tecentriq®) plus bevacizumab (Avastin®). Depending on the patient condition, at least one, at least two or at least three chemotherapeutic agents can be selected from the list above, to be combined with gevokizumab.

Immunotherapies currently in development have started to offer significant benefit to melanoma cancer patients, including those for whom conventional treatments are ineffective. Recently, pembrolizumab (Keytruda®) and nivolumab (Opdivo®), two inhibitors of the PD-1/PD-L1 interaction have been approved for use in melanoma. However, results indicate that many patients treated with single agent PD-1 inhibitors do not benefit adequately from treatment.

In one embodiment the one or more chemotherapeutic agent is nivolumab.

In one embodiment the one or more chemotherapeutic agent ipilimumab.

In one embodiment the one or more chemotherapeutic agent is nivolumab and ipilimumab.

In one embodiment the one or more chemotherapeutic agent is trametinib.

In one embodiment the one or more chemotherapeutic agent is Dabrafenib.

In one embodiment the one or more chemotherapeutic agent is trametinib and dabrafenib.

In one embodiment the one or more chemotherapeutic agent is Pembrolizumab.

In one embodiment the one or more chemotherapeutic agent is Atezolizumab.

In one embodiment the one or more chemotherapeutic agent is atezolizumab (Tecentriq®) plus bevacizumab.

In one embodiment, gevokizumab or a functional fragment thereof, alone or preferably in combination, is used in the prevention of recurrence or relapse of melanoma in a patient after said cancer has been surgically removed. In one embodiment, gevokizumab or a functional fragment thereof is used, alone or in preferably combination, in first line treatment of melanoma. In one embodiment gevokizumab or a functional fragment thereof is used, alone or in prefearbly combination, in second or third line of melanoma.

The above disclosed embodiments for gevokizumab or a functional fragment thereof are suitably applicable for canakinumab or a functional fragment thereof.

Like what has been observed concerning IL-1β in the development of lung cancer, it is plausible that IL-1β plays a similar role in the development of melanoma.

Tumor cells expressing the IL-1β precursor must first activate caspase-1 in order to process the inactive precursor into active cytokine. Activation of caspase-1 requires autocatalysis of procaspase-1 by the nucleotide-binding domain and leucine-rich repeat containing protein 3 (NLRP3) inflammasome (Dinarello, C. A. (2009). Ann Rev Immunol, 27, 519-550). In late-stage human melanoma cells, spontaneous secretion active IL-1β is observed via constitutive activation of the NLRP3 inflammasome (Okamoto, M. et al The Journal of Biological Chemistry, 285, 6477-6488). Unlike human blood monocytes, these melanoma cells require no exogenous stimulation. In contrast, NLRP3 functionality in intermediate stage melanoma cells requires activation of the IL-1 receptor by IL-1α in order to secrete active IL-1β. The spontaneous secretion of IL-1β from melanoma cells was reduced by inhibition of caspase-1 or the use of small interfering RNA directed against the inflammasome component ASC. Supernatants from melanoma cell cultures enhanced macrophage chemotaxis and promoted in vitro angiogenesis, both prevented by pretreating melanoma cells with inhibitors of caspases-1 or IL-1 receptor blockade (Okamoto, M. et al The Journal of Biological Chemistry, 285, 6477-6488). Furthermore, in a screen of human melanoma tumor samples, copy number greater than 1,000 for IL-1β was present in 14 of 16 biopsies, whereas none expressed IL-1a (Elaraj, D. M. et al, Clinical Cancer Research, 12, 1088-1096. Taken together these findings implicate IL-1-mediated autoinflammation, especially IL-1β, as contributing to the development and progression of human melanoma.

Thus in one aspect, the present invention provides an IL-1β binding antibody or a functional fragment thereof for use in the treatment and/or prevention of melanoma in a patient. In one embodiment, the patient has high sensitivity C-reactive protein (hsCRP) equal to or greater than 2 mg/L or equal to or greater than 4 mg/L.

In one embodiment, about 90 mg to about 450 mg of an IL-1β binding antibody or a functional fragment thereof in administered to melanoma patient per treatment, preferably every two, three or four weeks (monthly).

In one embodiment, the IL-1β binding antibody is canakinumab. Preferably 300 mg of canakinumab is administered monthly. Furthermore the second administration of canakinumab is at most two weeks, preferably two weeks apart from the first administration. furthermore canakinumab is administered subcutaneously. Furthermore canakinumab is administered in a liquid form contained in a prefilled syringe or as a lyophilized form for reconstitution.

In one embodiment the IL-1β binding antibody is gevokizumab (XOMA-052). Furthermore gevokizumab is administered subcutaneously or intravenously.

It is the data arisen from CANTOS that provided clinical evidence for the first time of the effectiveness of an IL-1β in the treatment of lung cancer, a cancer that has at least a partial inflammatory basis. Furthermore lung cancer has concomitant inflammation activated or mediated in part through activation of the Nod-like receptor protein 3 (NLRP3) inflammasome with consequent local production of interleukin-1β. It is plausible that melanoma shares similar mechanism in terms of the involvement of IL-1β in cancer development. Thus it is plausible that an IL-1β binding antibody or a functional fragment thereof, especially canakinumab, is effective in the treatment of melanoma.

All the teachings disclosed in the present application concerning the use of an IL-1 (3 binding antibody or a functional fragment thereof, especially canakinumab or gevokizumab, particularly regarding the dosing regimen of canakinumab or gevokizumab, particularly regarding the patients' hsCRP level and its reduction by the treatment, particularly regarding the use of hsCRP as biomarker, in the treatment and/or prevention of lung cancer are equally applicable or can be easily modified by a skilled person, in the treatment and/or prevention of melanoma.

In certain embodiments, the present invention provides an IL-1β antibody or a functional fragment thereof, suitably gevokizumab or a functional fragment thereof, suitably canakinumab or a functional fragment thereof, for use in the treatment of bladder cancer. The term “bladder cancer” as used herein refers to squamous cell carcinoma of the bladder, adenocarcinoma of the bladder, small cell carcinoma of the bladder and urothelial (cell) carcinoma, i.e. carcinomas of the urinary bladder, ureter, renal pelvis and urethra. The term includes reference to the non muscle-invasive (NMI) or superficial forms, as well as to the muscle invasive (MI) types. Also included in the term is reference to primary bladder cancer, locally advanced bladder cancer, unresectable bladder cancer, metastatic bladder cancer, refractory bladder cancer, relapsed bladder cancer and/or cancer drug resistant bladder cancer. All the disclosed uses throughout this application, including but not being limited to, doses and dosing regimens, combinations, route of administration and biomarkers can be applied to the treatment of bladder cancer. In one embodiment, canakinumab is administered at a dose of from 200 mg to 400 mg per treatment, wherein canakinumab is administered preferably every 3 weeks or preferably monthly. In one embodiment, canakinumab is administered at a dose of 200 mg every 3 weeks, preferably subcutaneously. In one embodiment, gevokizumab is administered at a dose of from 90 mg to 200 mg per treatment, wherein gevokizumab is administered preferably every 3 weeks or preferably monthly. In one embodiment, gevokizumab is administered at a dose of 120 mg every 3 weeks or monthly, preferably intravenously.

Treatment regimens of bladder cancer include intravesical therpy for early stages of bladder cancer as well as chemotherapy with and without radiation therapy.

In one embodiment, the present invention provides gevokizumab or a functional fragment thereof, for use in the treatment of bladder cancer, wherein gevokizumab, or a functional fragment thereof, is administered in combination with one or more chemotherapeutic agent. In one embodiment the chemotherapeutic agent is the standard of care agent for bladder cancer. In one embodiment the one or more chemotherapeutic agent is selected from cisplatin, cisplatin plus fluorouracil (5-FU), mitomycin plus 5-FU, gemcitabine plus cisplatin, MVAC (methotrexate, vinblastine, doxorubicin (adriamycin), plus cisplatin), CMV (cisplatin, methotrexate, and vinblastine), carboplatin plus paclitaxel or docetaxel, gemcitabine, cisplatin, carboplatin, docetaxel, paclitaxel, doxorubicin, 5-FU, methotrexate, vinblastine, ifosfamide, pemetrexed, thiotepa, valrubicin, atezolizumab (Tecentriq®), avelumab (Bavencio®), durvalumab (Imfinzi®), pembrolizumab (Keytruda®) and nivolumab (Opdivo®).

Depending on the patient condition, at least one, at least two or at least three chemotherapeutic agents can be selected from the list above, to be combined with gevokizumab.

In one embodiment the one or more chemotherapeutic agent is a checkpoint inhibitor, wherein preferably is a PD-1 or PD-L1 inhibitor, wherein preferably selected from the group consisting of nivolumab, pembrolizumab, atezolizumab, avelumab, durvalumab and spartalizumab (PDR-001).

In one embodiment, gevokizumab or a functional fragment thereof is used in the prevention of recurrence or relapse of bladder cancer in a patient after said cancer has been surgically removed. In one embodiment, gevokizumab or a functional fragment thereof is used in first line treatment of bladder cancer. In one embodiment gevokizumab or a functional fragment thereof is used in second or third line of bladder cancer.

The above disclosed embodiments for gevokizumab or a functional fragment thereof are suitably applicable for canakinumab or a functional fragment thereof.

In certain embodiments, the present invention provides an IL-1β antibody or a functional fragment thereof, suitably gevokizumab or a functional fragment thereof, suitably canakinumab or a functional fragment thereof, for use in the treatment of prostate cancer. The terra “prostate cancer” as used herein, refers to acinar adenocarcinoma, ductal adenocarcinoma, squamous cell prostate cancer, small cell prostate cancer and includes androgen-deprivation/castration-sensitive prostate cancer, androgen-deprivation/castration-resistant prostate cancer, primary prostate cancer, locally advanced prostate cancer, unresectable: prostate cancer, metastatic prostate cancer, refractory prostate cancer, relapsed prostate cancer and/or cancer drug resistant prostate cancer.

All the disclosed uses throughout this application, including but not limited to, doses and dosing regimens, combinations, route of administration and biomarkers can be applied to the treatment of prostate cancer. In one embodiment, canakinumab is administered at a dose of from 200 mg to 400 mg per treatment, wherein canakinumab is administered preferably every 3 weeks or preferably monthly. In one embodiment, canakinumab is administered at a dose of 200 mg every 3 weeks, preferably subcutaneously. In one embodiment, gevokizumab is administered at a dose of from 90 mg to 200 mg per treatment, wherein gevokizumab is administered preferably every 3 weeks or preferably monthly. In one embodiment, gevokizumab is administered at a dose of 120 mg every 3 weeks or monthly, preferably intravenously.

In one embodiment, the present invention provides gevokizumab or a functional fragment thereof, for use in the treatment of prostate cancer, wherein gevokizumab, or a functional fragment thereof, is administered in combination with one or more chemotherapeutic agent. In one embodiment the chemotherapeutic agent is the standard of care agent for prostate cancer. In one embodiment the one or more chemotherapeutic agent is selected from abiraterone, apalutamide, bicalutamide, cabazitaxel, degarelix, docetaxel, docetaxel plus prednisone, enzalutamide (Xtandi®), flutamide, goserelin acetate, leuprolide acetate, ketoconazole, aminoglutethamide, mitoxantrone hydrochloride, nilutamide, sipuleucel-T, radium 223 dichloride, estramustine, rilimogene galvacirepvec/rilimogene glafolivec (PROSTVAC®), pembrolizumab (Keytruda®), pembrolizumab plus enzalutamide.

Depending on the patient condition, at least one, at least two or at least three chemotherapeutic agents can be selected from the list above, to be combined with gevokizumab.

In one embodiment the one or more chemotherapeutic agent is a checkpoint inhibitor, wherein preferably is a PD-1 or PD-L1 inhibitor, wherein preferably selected from the group consisting of nivolumab, pembrolizumab, atezolizumab, avelumab, durvalumab and spartalizumab (PDR-001).

In one embodiment, gevokizumab or a functional fragment thereof is used in the prevention of recurrence or relapse of prostate cancer in a patient after said cancer has been surgically removed. In one embodiment, gevokizumab or a functional fragment thereof is used in first line treatment of prostate cancer. In one embodiment gevokizumab or a functional fragment thereof is used in second or third line of prostate cancer.

The above disclosed embodiments for gevokizumab or a functional fragment thereof are suitably applicable for canakinumab or a functional fragment thereof.

In certain embodiments, the present invention provides an IL-1β antibody or a functional fragment thereof, suitably gevokizumab or a functional fragment thereof, suitably canakinumab or a functional fragment thereof, for use in the treatment of breast cancer. The term “breast cancer” as used herein includes breast cancer arising in ducts (ductal carcinoma, including invasive ductal carcinoma and ductal carcinoma in situ (DCIS)), glands (lobular carcinoma, including Invasive lobular carcinoma, and lobular carcinoma in situ (LCIS), inflammatory breast cancer, angiosarcoma; and including but not limited to, estrogen-receptor-positive (ER+) breast cancer, progesterone-receptor-positive (PR+) breast cancer, herceptin-receptor positive (HER2+) breast cancer, herceptin-receptor negative (HER2−) breast cancer, ER-positive/HER2-negative breast cancer and triple negative breast cancer (TNBC; a breast cancer that is HER2−, ER− and PR−).

All the disclosed uses throughout this application, including but not limited to, doses and dosing regimens, combinations, route of administration and biomarkers can be applied to the treatment of breast cancer. In one embodiment, canakinumab is administered at a dose of from 200 mg to 400 mg per treatment, wherein canakinumab is administered preferably every 3 weeks or preferably monthly. In one embodiment, canakinumab is administered at a dose of 200 mg every 3 weeks, preferably subcutaneously. In one embodiment, gevokizumab is administered at a dose of from 90 mg to 200 mg per treatment, wherein gevokizumab is administered preferably every 3 weeks or preferably monthly. In one embodiment, gevokizumab is administered at a dose of 120 mg every 3 weeks or monthly, preferably intravenously.

Treatment regimens of breast cancer include intravesical therapy for early stages of breast cancer as well as chemotherapy with and without radiation therapy.

In one embodiment, the present invention provides gevokizumab or a functional fragment thereof, for use in the treatment of breast cancer, wherein gevokizumab, or a functional fragment thereof, is administered in combination with one or more chemotherapeutic agent. In one embodiment the chemotherapeutic agent is the standard of care agent for breast cancer. In one embodiment the one or more chemotherapeutic agent is selected from abemaciclib, methotrexate, abraxane (paclitaxel albumin-stabilized nanoparticle formulation), ado-trastuzumab emtansine, anastrozole, pamidronate disodiumrozole, capecitabine, cyclophosphamide, docetaxel, doxorubicin hydrochloride, epirubicin hydrochloride, eribulin mesylate, exemestane, fluorouracil injection, fulvestrant, gemcitabine hydrochloride, goserelin acetate, ixabepilone, lapatinib ditosylate, letrozole, megestrol acetate, methotrexate, neratinib maleate, olaparib, paclitaxel, pamidronate disodium, tamoxifen, thiotepa, toremifene, vinblastine sulfate, AC (doxorubicin hydrochloride (adriamycin) and cyclophosphamide), AC-T (doxorubicin hydrochloride (adriamycin), cyclophosphamide and paclitaxel), CAF (cyclophosphamide, doxorubicin hydrochloride (adriamycin) and fluorouracil), CMF (cyclophosphamide, methotrexate and fluorouracil), FEC (fluorouracil, epirubicin hydrochloride, cyclophosphamide), TAC (docetaxel (taxotere), doxorubicin hydrochloride (adriamycin), cyclophosphamide), palbociclib, abemaciclib, ribociclib, everolimus, trastuzumab (Herceptin®), ado-trastuzumab emtansine (Kadcyla®), vorinostat (Zolinza®), romidepsin (Istodax®), chidamide (Epidaza®), panobinostat (Farydak®), belinostat (Beleodaq®, pxd101), valproic acid (Depakote®, Depakene®, Stavzor®), mocetinostat (mgcd0103), abexinostat (pci-24781), entinostat (ms-275), pracinostat (sb939), resminostat (4sc-201), givinostat (itf2357), quisinostat (jnj-26481585), kevetnn, cudc-101, ar-42, tefinostat (chr-2835), chr-3996, 4sc202, cg200745, rocilinostat (acy-1215), sulforaphane, or a checkpoint inhibitor such as nivolumab, pembrolizumab, atezolizumab, avelumab, durvalumab spartalizumab (PDR-001), and ipilimumab.

Depending on the patient condition, at least one, at least two or at least three chemotherapeutic agents can be selected from the list above, to be combined with gevokizumab.

In one embodiment the one or more chemotherapeutic agent is a checkpoint inhibitor, wherein preferably is a PD-1 or PD-L1 inhibitor, wherein preferably selected from the group consisting of nivolumab, pembrolizumab, atezolizumab, avelumab, durvalumab and spartalizumab (PDR-001).

In one preferred embodiment IL-1β antibody or a functional fragment thereof, preferably canakinumab or gevokizumab, is used in combination of one or more chemotherapeutic agents, wherein said agent is an anti-Wnt inhibitor, preferably Vantictumab. This embodiment is particularly useful in the inhibition of breast tumor metastasis.

In one embodiment, gevokizumab or a functional fragment thereof is used, alone or preferably in combination, in the prevention of recurrence or relapse of breast cancer in a patient after said cancer has been surgically removed. In one embodiment, gevokizumab or a functional fragment thereof is use, alone or preferably in combination, in first line treatment of breast cancer. In one embodiment gevokizumab or a functional fragment thereof is used, alone or preferably in combination, in second or third line of breast cancer. In one embodiment, gevokizumab or a functional fragment thereof is used, alone or preferably in combination, in the treatment of TNBC.

The above disclosed embodiments for gevokizumab or a functional fragment thereof are suitably applicable for canakinumab or a functional fragment thereof.

In certain embodiments, the present invention provides an IL-1β antibody or a functional fragment thereof, suitably gevokizumab or a functional fragment thereof, suitably canakinumab or a functional fragment thereof, for use in the treatment of pancreatic cancer.

As used herein, the term “pancreatic cancer” refers to pancreatic endocrine and pancreatic exocrine tumors and includes adenocarcinoma arising from pancreatic ductal epithelium, suitably pancreatic ductal adenocarcinoma (PDAC) or a neoplasm arising from pancreatic islet cells and includes pancreatic neuroendocrine tumors (pNETs) such as gastrinoma, insulinoma, glucagonoma, VIPomas and somatostatinomas. The pancreatic cancer may be primary pancreatic cancer, locally advanced pancreatic cancer, unresectable pancreatic cancer, metastatic pancreatic cancer, refractory pancreatic cancer, and/or cancer drug resistant pancreatic cancer.

All the disclosed uses throughout this application, including but not limited to, doses and dosing regimens, combinations, route of administration and biomarkers can be applied to the treatment of pancreatic cancer. In one embodiment, canakinumab is administered at a dose of from 200 mg to 400 mg per treatment, wherein canakinumab is administered preferably every 3 weeks or preferably monthly. In one embodiment, canakinumab is administered at a dose of 200 mg every 3 weeks, preferably subcutaneously. In one embodiment, gevokizumab is administered at a dose of from 90 mg to 200 mg per treatment, wherein gevokizumab is administered preferably every 3 weeks or preferably monthly. In one embodiment, gevokizumab is administered at a dose of 120 mg every 3 weeks or monthly, preferably intravenously.

In one embodiment, the present invention provides gevokizumab or a functional fragment thereof, for use in the treatment of pancreatic cancer, wherein gevokizumab, or a functional fragment thereof, is administered in combination with one or more chemotherapeutic agent. In one embodiment the chemotherapeutic agent is the standard of care agent for gastric cancer. In one embodiment the one or more chemotherapeutic agent is selected from nab-paclitaxel (Paclitaxel Albumin-stabilized Nanoparticle Formulation; Abraxane®), docetaxel, capecitabine, everolimus (Afinitor®), erlotinib hydrochloride (Tarceva®), sunitinib malate (Sutent®), fluorouracil (5-FU), gemcitabine hydrochloride, irinotecan, mitomycin C, FOLFIRINOX (leucovorin calcium (folinic acid), fluorouracil, irinotecan hydrochloride and oxaliplatin), gemcitabine plus cisplatin, gemcitabine plus oxaliplatin, gemcitabine plus nab-paclitaxel, and OFF (oxaliplatin, fluorouracil and leucovorin calcium (folinic acid)). Depending on the patient condition, at least one, at least two or at least three chemotherapeutic agents can be selected from the list above, to be combined with gevokizumab.

In one embodiment the one or more chemotherapeutic agent is a checkpoint inhibitor, wherein preferably is a PD-1 or PD-L1 inhibitor, wherein preferably selected from the group consisting of nivolumab, pembrolizumab, atezolizumab, avelumab, durvalumab and spartalizumab (PDR-001).

In one embodiment, gevokizumab or a functional fragment thereof is used, alone or preferably in combination, in the prevention of recurrence or relapse of pancreatic cancer in a patient after said cancer has been surgically removed. In one embodiment, gevokizumab or a functional fragment thereof is used, alone or preferably in combination, in first line treatment of pancreatic cancer. In one embodiment gevokizumab or a functional fragment thereof is used, alone or preferably in combination, in second or third line of pancreatic cancer.

The above disclosed embodiments for gevokizumab or a functional fragment thereof are suitably applicable for canakinumab or a functional fragment thereof.

In one aspect, the present invention provides a pharmaceutical composition comprising an IL-1β binding antibody or a functional fragment thereof and at least one pharmaceutically acceptable carrier for use in the treatment and/or prevention of cancer having at least a partial inflammatory basis, including lung cancer in a patient. Preferably the pharmaceutical composition comprises a therapeutically effective amount of IL-1β binding antibody or a functional fragment thereof.

In one aspect of this invention canakinumab or a functional fragment thereof is administered intravenously. In one aspect of this invention canakinumab or a functional fragment thereof is preferably administered subcutaneously.

In one aspect of this invention gevokizumab or a functional fragment thereof is administered subcutaneously. In one aspect of this invention gevokizumab or a functional fragment thereof is preferably administered intravenously.

Canakinumab can be administered in a reconstituted formulation comprising comprising canakinumab at a concentration of 50-200 mg/ml, 50-300 mM sucrose, 10-50 mM histidine, and 0.01-0.1% surfactant and wherein the pH of the formulation is 5.5-7.0. Canakinumab can be administered in a reconstituted formulation comprising canakinumab at a concentration of 50-200 mg/ml, 270 mM sucrose, 30 mM histidine and 0.06% polysorbate 20 or 80, wherein the pH of the formulation is 6.5.

Canakinumab can also be administered in a liquid formulation comprising canakinumab at a concentration of 50-200 mg/ml, a buffer system selected from the group consisting of citrate, histidine and sodium succinate, a stabilizer selected from the group consisting of sucrose, mannitol, sorbitol, arginine hydrochloride, and a surfactant and wherein the pH of the formulation is 5.5-7.0. Canakinumab can also be administered in a liquid formulation comprising canakinumab at a concentration of 50-200 mg/ml, 50-300 mM mannitol, 10-50 mM histidine and 0.01-0.1% surfactant, and wherein the pH of the formulation is 5.5-7.0. Canakinumab can also be administered in a liquid formulation comprising canakinumab at a concentration of 50-200 mg/ml, 270 mM mannitol, 20 mM histidine and 0.04% polysorbate 20 or 80, wherein the pH of the formulation is 6.5.

When administered subcutaneously, canakinumab can be administered to the patient in a liquid form contained in a prefilled syringe or as a lyophilized form for reconstitution.

In one aspect, the present invention provides high sensitivity C-reactive protein (hsCRP) for use as a biomarker in the treatment and/or prevention of cancer having at least a partial inflammatory basis, including lung cancer, with an IL-1β inhibitor, IL-1β binding antibody or a functional fragment thereof. Typically cancers that have at least a partial inflammatory basis include but are not limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer. Consistent with prior work indicating a strong inflammatory component to certain cancers, hsCRP levels in the CANTOS trial population were elevated at baseline among those who were diagnosed with lung cancer during follow-up compared to those who remained free of any cancer diagnosis (6.0 versus 4.2 mg/L, P<0.001). Thus the level of hsCRP is possibly relevant in determining whether a patient with diagnosed lung cancer, undiagnosed lung cancer or is at risk of developing lung cancer should be treated with an IL-1β inhibitor, IL-1β binding antibody or a functional fragment thereof. In a preferred embodiment, said IL-1β binding antibody or a fragment thereof is canakinumab or a fragment thereof or gevokizumab or a fragment thereof. Similarly the level of hsCRP is possibly relevant in determining whether a patient with cancer having at least a partial inflammatory basis, diagnosed or undiagnosed, should be treated with an IL-1β inhibitor, IL-1β binding antibody or a functional fragment thereof. In a preferred embodiment, said IL-1β binding antibody is canakinumab or gevokizumab.

Thus the present invention provides high sensitivity C-reactive protein (hsCRP) for use as a biomarker in the treatment and/or prevention of cancer having at least a partial inflammatory basis, including lung cancer, in a patient with an IL-1β inhibitor, IL-1β binding antibody or a functional fragment thereof, wherein said patient is eligible for the treatment and/or prevention if the level of high sensitivity C-reactive protein (hsCRP) is equal to or higher than 2 mg/L, or equal to or higher than 3 mg/L, or equal to or higher than 4 mg/L, or equal to or higher than 5 mg/L, or equal to or higher than 6 mg/L, equal to or higher than 7 mg/L, equal to or higher than 8 mg/L, equal to or higher than 9 mg/L, or equal to or higher than 10 mg/L, equal to or higher than 12 mg/L, equal to or higher than 15 mg/L, equal to or higher than 20 mg/L or equal to or higher than 25 mg/L as assessed prior to the administration of the IL-1β binding antibody or a functional fragment thereof. In a preferred embodiment, said patient has hsCRP level equal to or higher than 4 mg/L. In a preferred embodiment, said patient has hsCRP level equal to or higher than 6 mg/L. In a preferred embodiment, said patient has hsCRP level equal to or higher than 10 mg/L.

In analyses of combined canakinumab doses, compared to placebo, the observed hazard ratio for lung cancer among those who achieved hsCRP reductions greater than the median value of 1.8 mg/L at 3 months was 0.29 (95% CI 0.17-0.51, P<0.0001), better than the effect observed for those who achieved hsCRP reductions less than the median value (HR 0.83, 95% CI 0.56-1.22, P=0.34).

Thus in one aspect, the present invention relates to the use of the degree of reduction of the hsCRP as a prognostic biomarker to guide physician in continuing or discontinuing with the treatment of an IL-1β inhibitor, an IL-1β binding antibody or a functional fragment thereof, especially canakinumab or gevokizumab. In one embodiment, the present invention provides the use of an IL-1β inhibitor, an IL-1β binding antibody or a functional fragment thereof, in the treatment and/or prevention of cancer having at least a partial inflammatory basis, including lung cancer, wherein such treatment or prevention is continued when the level of hsCRP is reduced by at least 0.8 mg/L, at least 1 mg/L, at least 1.2 mg/L, at least 1.4 mg/L, at least 1.6 mg/L, at least 1.8 mg/L, at least 3 mg/L or at least 4 mg/L, at least 3 months, preferably 3 months after first administration of the IL-1β binding antibody or functional fragment thereof. In one embodiment, the present invention provides the use of an IL-1β inhibitor, IL-1β binding antibody or a functional fragment thereof, in the treatment and/or prevention of cancer having at least a partial inflammatory basis, including lung cancer, wherein such treatment or prevention is discontinued when the level of hsCRP is reduced by less than 0.8 mg/L, less than 1 mg/L, less than 1.2 mg/L, less than 1.4 mg/L, less than 1.6 mg/L, less than 1.8 mg/L at about 3 months from the beginning of the treatment at an appropriate dosing with the IL-1β binding antibody or functional fragment thereof. In a further embodiment the appropriate dosing of canakinumab is 50 mg, 150 mg or 300 mg, which is administered every 3 months. In a further embodiment the appropriate dosing of canakinumab is 300 mg administered twice over a two-week period and then every three months. In one embodiment, the IL-1β binding antibody or a functional fragment thereof is canakinumab or a functional fragment thereof, wherein said canakinumab is administered at a dose of 200 mg every 3 weeks or 200 mg monthly. In one embodiment, the IL-1β binding antibody or a functional fragment thereof is gevokizumab or a functional fragment thereof, wherein said gevokizumab is administered at a dose of 60 mg to 90 mg or 120 mg every 3 weeks or monthly.

In one aspect, the present invention provides the use of the reduced hsCRP level as a prognostic biomarker to guide a physician in continuing or discontinuing with the treatment of an IL-1β binding antibody or a functional fragment thereof, especially canakinumab or gevokizumab. In one embodiment, such treatment and/or prevention with the IL-1β binding antibody or a functional fragment thereof is continued when the level of hsCRP is reduced below 10 mg/L, reduced below 8 mg/L, reduced below 5 mg/L, reduced below 3.5 mg/L, below 3 mg/L, below 2.3 mg/L, below 2 mg/L or below 1.8 mg/L assessed at least 3 months from first administration of the IL-1β binding antibody or a functional fragment thereof. In one embodiment, such treatment and/or prevention with the IL-1β binding antibody or a functional fragment thereof is discontinued when the level of hsCRP is not reduced below 3.5 mg/ml, below 3 mg/L, below 2.3 mg/L, below 2 mg/L or below 1.8 mg/L assessed at least 3 months from first administration of the IL-1β binding antibody or a functional fragment thereof. In a further embodiment the appropriate dosing is canakinumab at 300 mg administered twice over a two-week period and then every three months. In one embodiment, the IL-1β binding antibody or a functional fragment thereof is canakinumab or a functional fragment thereof, wherein said canakinumab is administered at a dose of 200 mg every 3 weeks or 200 mg monthly or 300 mg monthly. In one embodiment, the IL-1β binding antibody or a functional fragment thereof is gevokizumab or a functional fragment thereof, wherein said gevokizumab is administered at a dose of 60 mg to 90 mg or 120 mg every 3 weeks or monthly.

In one aspect, the present invention provides an IL-1β binding antibody or a functional fragment thereof for use in a patient in need thereof in the treatment of a cancer having at least partial inflammatory basis, wherein said IL-1β binding antibody or a functional fragment thereof is administered at a dose sufficient to inhibit angiogenesis in said patient. Without wishing to be bound by theory, it is hypothesized that the inhibition of IL-1β pathway can lead to inhibition or reduction of angiogenesis, which is a key event for tumor growth and for tumor metastasis. Thus in clinical settings the inhibition of angiogenesis can be measued by tumor shrinkage, no tumor growth (stable disease), prevention of metastasis or delay of metastasis. Typically cancer having at least partial inflammatory basis includes but is not limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, multiple myeloma and pancreatic cancer.

In one embodiment said cancer is lung cancer, especially NSCLC. In one embodiment said cancer is breast cancer. In one embodiment said cancer is colorectal cancer. In one embodiment said cancer is gastric cancer. In one embodiment said cancer is renal carcinoma. In one embodiment said cancer is melanoma.

In one embodiment said dose sufficient to inhibit angiogenesis comprises an IL-1β binding antibody or a functional fragment thereof to be administered in the range of about 30 mg to about 750 mg per treatment, alternatively 100 mg-600 mg, 100 mg to 450 mg, 100 mg to 300 mg, alternatively 150 mg-600 mg, 150 mg to 450 mg, 150 mg to 300 mg, preferably 150 mg to 300 mg; alternatively at least 150 mg, at least 180 mg, at least 250 mg, at least 300 mg per treatment. In one embodiment the patient with a cancer that has at least a partial inflammatory basis, including lung cancer, receives each treatment every 2 weeks, every three weeks, every four weeks (monthly), every 6 weeks, bimonthly (every 2 months) or quarterly (every 3 months). In one embodiment the range of DRUG of the invention is 90 mg to 450 mg. In one embodiment said DRUG of the invention is administered monthly. In one embodiment said DRUG of the invention is administered every 3 weeks.

In one embodiment, the IL-1β binding antibody is canakinumab administered at a dose sufficient to inhibit angiogenesis, wherein said dose is in the range of about 100 mg to about 750 mg per treatment, alternatively 100 mg-600 mg, 100 mg to 450 mg, 100 mg to 300 mg, alternatively 150 mg-600 mg, 150 mg to 450 mg, 150 mg to 300 mg, alternatively at least 150 mg, at least 200 mg, at least 250 mg, at least 300 mg per treatment. In one embodiment the patient with cancer having at least a partial inflammatory basis, including lung cancer, receives each treatment every 2 weeks, every 3 weeks, every 4 weeks (monthly), every 6 weeks, bimonthly (every 2 months) or quarterly (every 3 months). In one embodiment the patient with lung cancer receives canakinumab monthly. In one embodiment the preferred dose range of canakinumab is 200 mg to 450 mg, further preferred 300 mg to 450 mg, further preferred 350 mg to 450 mg. In one embodiment the preferred dose range of canakinumab is 200 mg to 450 mg every 3 weeks or monthly. In one embodiment the preferred dose of canakinumab is 200 mg every 3 weeks. In one embodiment the preferred dose of canakinumab is 200 mg monthly. In one embodiment canakinumab is administered subcutaneously or intravenously, prefearbly subcutaneously.

In one embodiment, the IL-1β binding antibody is gevokizumab administered at a dose sufficient to inhibit angiogenesis, wherein said dose is in the range of about 30 mg to about 450 mg per treatment, alternatively 90 mg-450 mg, 90 mg to 360 mg, 90 mg to 270 mg, 90 mg to 180 mg; alternatively 120 mg-450 mg, 120 mg to 360 mg, 120 mg to 270 mg, 120 mg to 180 mg, alternatively 150 mg-450 mg, 150 mg to 360 mg, 150 mg to 270 mg, 150 mg to 180 mg; alternatively 180 mg-450 mg, 180 mg to 360 mg, 180 mg to 270 mg; alternatively at least 150 mg, at least 180 mg, at least 240 mg, at least 270 mg per treatment. In one embodiment the patient with cancer that has at least a partial inflammatory basis, including lung cancer, receives treatment every 2 weeks, every 3 weeks, monthly, every 6 weeks, bimonthly (every 2 months) or quarterly (every 3 months). In one embodiment the patient with cancer that has at least a partial inflammatory basis, including lung cancer, receives at least one, preferably one treatment per month. In one embodiment the preferred range of gevokizumab is 150 mg to 270 mg. In one embodiment the preferred range of gevokizumab is 60 mg to 180 mg, further preferred 60 mg to 90 mg. In one embodiment the preferred schedule is every 3 weeks. In one embodiment the preferred schedule is monthly. In one embodiment the patient receives gevokizumab 60 mg to 90 mg every 3 weeks. In one embodiment the patient receives gevokizumab 60 mg to 90 mg monthly. In one embodiment the patient with cancer that has at least a partial inflammatory basis receives gevokizumab about 90 mg to about 360 mg, 90 mg to about 270 mg, 120 mg to 270 mg, 90 mg to 180 mg, 120 mg to 180 mg, 120 mg or 90 mg every 3 weeks. In one embodiment the patient with cancer that has at least a partial inflammatory basis receives gevokizumab about 90 mg to about 360 mg, 90 mg to about 270 mg, 120 mg to 270 mg, 90 mg to 180 mg, 120 mg to 180 mg, 120 mg or 90 mg monthly. In one embodiment the patient receives gevokizumab 90 mg, every 180 mg, 190 mg or 200 mg every 3 weeks. In one embodiment the patient receives gevokizumab 90 mg, every 180 mg, 190 mg or 200 mg monthly. In one embodiment the patient receives gevokizumab 120 mg monthly or every 3 weeks. In one embodiment gevokizumab is administered subcutaneously or intravenously, preferably intravenously.

All the disclosed uses throughout this application, including but not limited to, doses and dosing regimens, combinations, route of administration and biomarkers can be applied to the embodiment of angiogenesis inhibition. In one preferred embodiment IL-1β antibody or a functional fragment thereof is used in combination of one or more chemotherapeutic agents, wherein said agent is an anti-Wnt inhibitor, prefearbly Vantictumab.

Without wishing to be being bound by theory, it is hypothesized that the inhibition of IL-1β pathway can lead to inhibition or reduction of tumor metastasis. Until now there have been no reports on the effects of canakinumab on metastasis. Data presented in example 3 demonstrate that IL-1β activates different pro-metastatic mechanisms at the primary site compared with the metastatic site: Endogenous production of IL-1β by breast cancer cells promotes epithelial to mesenchymal transition (EMT), invasion, migration and organ specific homing. Once tumor cells arrive in the bone environment contact between tumor cells and osteoblasts or bone marrow cells increase IL-1β secretion from all three cell types. These high concentrations of IL-1β cause proliferation of the bone metastatic niche by stimulating growth of disseminated tumor cells into overt metastases. These pro-metastatic processes are inhibited by administration of anti-IL-1β treatments, such as canakinumab.

Therefore, targeting IL-1β with an IL-1β binding antibody represents a novel therapeutic approach for cancer patients at risk of progressing to metastasis by preventing seeding of new metastases from established tumors and retaining tumor cells already disseminated in the bone in a state of dormancy. The models described have been designed to investigate bone metastasis and although the data show a strong link between IL-1β expression and bone homing, it does not exclude IL-1β involvement in metastasis to other sites.

Accordingly, in one aspect, the present invention provides an IL-1β binding antibody or a functional fragment thereof for use in a patient in need thereof in the treatment of a cancer having at least partial inflammatory basis, wherein said IL-1β binding antibody or a functional fragment thereof is administered at a dose sufficient to inhibit metastasis in said patient. Typically cancer having at least partial inflammatory basis includes but is not limited to lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, multiple myeloma and pancreatic cancer.

In one embodiment said dose sufficient to inhibit metastasis comprises an IL-1β binding antibody or a functional fragment thereof to be administered in the range of about 30 mg to about 750 mg per treatment, alternatively 100 mg-600 mg, 100 mg to 450 mg, 100 mg to 300 mg, alternatively 150 mg-600 mg, 150 mg to 450 mg, 150 mg to 300 mg, preferably 150 mg to 300 mg; alternatively at least 150 mg, at least 180 mg, at least 250 mg, at least 300 mg per treatment. In one embodiment the patient with a cancer that has at least a partial inflammatory basis, including lung cancer, receives each treatment every 2 weeks, every three weeks, every four weeks (monthly), every 6 weeks, bimonthly (every 2 months) or quarterly (every 3 months). In one embodiment the range of DRUG of the invention is 90 mg to 450 mg. In one embodiment said DRUG of the invention is administered monthly. In one embodiment said DRUG of the invention is administered every 3 weeks.

In one embodiment the IL-1β binding antibody is canakinumab administered at a dose sufficient to inhibit metastasis, wherein said dose is in the range of about 100 mg to about 750 mg per treatment, alternatively 100 mg-600 mg, 100 mg to 450 mg, 100 mg to 300 mg, alternatively 150 mg-600 mg, 150 mg to 450 mg, 150 mg to 300 mg, alternatively at least 150 mg, at least 200 mg, at least 250 mg, at least 300 mg per treatment. In one embodiment the patient with cancer having at least a partial inflammatory basis, including lung cancer, receives each treatment every 2 weeks, every 3 weeks, every 4 weeks (monthly), every 6 weeks, bimonthly (every 2 months) or quarterly (every 3 months). In one embodiment the patient with cancer receives canakinumab monthly. In one embodiment the preferred dose range of canakinumab is 200 mg to 450 mg, further preferred 300 mg to 450 mg, further preferred 350 mg to 450 mg. In one embodiment the preferred dose range of canakinumab is 200 mg to 450 mg every 3 weeks or monthly. In one embodiment the preferred dose of canakinumab is 200 mg every 3 weeks. In one embodiment the preferred dose of canakinumab is 200 mg monthly. In one embodiment canakinumab is administered subcutaneously or intravenously, preferably subcutaneously.

In one embodiment, the IL-1β binding antibody is gevokizumab administered at a dose sufficient to inhibit metastasis, wherein said dose is in the range of about 30 mg to about 450 mg per treatment, alternatively 90 mg-450 mg, 90 mg to 360 mg, 90 mg to 270 mg, 90 mg to 180 mg; alternatively 120 mg-450 mg, 120 mg to 360 mg, 120 mg to 270 mg, 120 mg to 180 mg, alternatively 150 mg-450 mg, 150 mg to 360 mg, 150 mg to 270 mg, 150 mg to 180 mg; alternatively 180 mg-450 mg, 180 mg to 360 mg, 180 mg to 270 mg; alternatively at least 150 mg, at least 180 mg, at least 240 mg, at least 270 mg per treatment. In one embodiment the patient with cancer that has at least a partial inflammatory basis, including lung cancer, receives treatment every 2 weeks, every 3 weeks, monthly, every 6 weeks, bimonthly (every 2 months) or quarterly (every 3 months). In one embodiment the patient with cancer that has at least a partial inflammatory basis, including lung cancer, receives at least one, preferably one treatment per month. In one embodiment the preferred range of gevokizumab is 150 mg to 270 mg. In one embodiment the preferred range of gevokizumab is 60 mg to 180 mg, further preferred 60 mg to 90 mg. In one embodiment the preferred schedule is every 3 weeks. In one embodiment the preferred schedule is monthly. In one embodiment the patient receives gevokizumab 60 mg to 90 mg every 3 weeks. In one embodiment the patient receives gevokizumab 60 mg to 90 mg monthly. In one embodiment the patient with cancer that has at least a partial inflammatory basis receives gevokizumab about 90 mg to about 360 mg, 90 mg to about 270 mg, 120 mg to 270 mg, 90 mg to 180 mg, 120 mg to 180 mg, 120 mg or 90 mg every 3 weeks. In one embodiment the patient with cancer that has at least a partial inflammatory basis receives gevokizumab about 90 mg to about 360 mg, 90 mg to about 270 mg, 120 mg to 270 mg, 90 mg to 180 mg, 120 mg to 180 mg, 120 mg or 90 mg monthly. In one embodiment the patient receives gevokizumab 90 mg, every 180 mg, 190 mg or 200 mg every 3 weeks. In one embodiment the patient receives gevokizumab 90 mg, every 180 mg, 190 mg or 200 mg monthly. In one embodiment the patient receives gevokizumab 120 mg monthly or every 3 weeks. In one embodiment gevokizumab is administered subcutaneously or intravenously, preferably intravenously.

All the disclosed uses throughout this application, including but not limited to, doses and dosing regimens, combinations, route of administration and biomarkers can be applied to the embodiment of metastasis inhibition. In one preferred embodiment IL-1β antibody or a functional fragment thereof is used in combination of one or more chemotherapeutic agents, wherein said agent is an anti-Wnt inhibitor, preferably Vantictumab.

IL-1β is known to drive the induction of gene expression of a variety of pro-inflammatory cytokines, such as IL-6 and TNF-α. In the CANTOS trial, it was observed that administration of canakinumab was associated with dose-dependent reductions in IL-6 of 25 to 43 percent (all P-values<0.0001). The present invention therefore also provides an IL-6 inhibitor for use in the treatment and/or prevention of cancer having at least a partial inflammatory basis, including but not limited to lung cancer. In some embodiments, the IL-6 inhibitor is selected from the group consisting of: anti-sense oligonucleotides against IL-6, IL-6 antibodies such as siltuximab (Sylvant®), sirukumab, clazakizumab, olokizumab, elsilimomab, gerilimzumab, WBP216 (also known as MEDI 5117), or a fragment thereof, EBI-031 (Eleven Biotherapeutics), FB-704A (Fountain BioPharma Inc), OP-R003 (Vaccinex Inc), IG61, BE-8, PPV-06 (Peptinov), SBP002 (Solbec), Trabectedin (Yondelis®), C326/AMG-220, olamkicept, PGE1 and its derivatives, PGI2 and its derivatives, and cyclophosphamide. Another embodiment of the present invention provides an IL-6 receptor (IL-6R) (CD126) inhibitor for use in the treatment and/or prevention of cancer having at least a partial inflammatory basis, including lung cancer. In some embodiments, the IL-6R inhibitor is selected from the group consisting of: anti-sense oligonucleotides against IL-6R, tocilizumab (Actemra®), sarilumab (Kevzara®), vobarilizumab, PM1, AUK12-20, AUK64-7, AUK146-15, MRA, satralizumab, SL-1026 (SomaLogic), LTA-001 (Common Pharma), BCD-089 (Biocad Ltd), APX007 (Apexigen/Epitomics), TZLS-501 (Novimmune), LMT-28, anti-IL-6R antibodies disclosed in WO2007143168 and WO2012118813, Madindoline A, Madindoline B, and AB-227-NA.

As used herein, canakinumab is defined under INN number 8836 and has the following sequence:

Light chain
(SEQ ID NO: 1007)

| | |
|---|---|
| 1 | EIVLTQSPDF QSVTPKEKVT ITCRASQSIG SSLHWYQQKP |
| | DQSPKLLIKY ASQSFSGVPS |
| 61 | RFSGSGSGTD FTLTINSLEA EDAAAYYCHQ SSSLPFTFGP |
| | GTKVDIKRTV AAPSVFIFPP |
| 121 | SDEQLKSGTA SVVCLLNNFY PREAKVQWKV DNALQSGNSQ |
| | ESVTEQDSKD STYSLSSTLT |
| 181 | LSKADYEKHK VYACEVTHQG LSSPVTKSFN RGEC* |

Heavy chain:
(SEQ ID NO: 1008)

| | |
|---|---|
| 1 | QVQLVESGGG VVQPGRSLRL SCAASGFTFS VYGMNWVRQA |
| | PGKGLEWVAI IWYDGDNQYY |
| 61 | ADSVKGRFTI SRDNSKNTLY LQMNGLRAED TAVYYCARDL |
| | RTGPFDYWGQ GTLVTVSSAS |
| 121 | TKGPSVFPLA PSSKSTSGGT AALGCLVKDY FPEPVTVSWN |
| | SGALTSGVHT FPAVLQSSGL |
| 181 | YSLSSVVTVP SSSLGTQTYI CNVNHKPSNT KVDKRVEPKS |
| | CDKTHTCPPC PAPELLGGPS |
| 241 | VFLFPPKPKD TLMISRTPEV TCVVVDVSHE DPEVKFNWYV |
| | DGVEVHNAKT KPREEQYNST |
| 301 | YRVVSVLTVL HQDWLNGKEY KCKVSNKALP APIEKTISKA |
| | KGQPREPQVY TLPPSREEMT |
| 361 | KNQVSLTCLV KGFYPSDIAV EWESNGQPEN NYKTTPPVLD |
| | SDGSFFLYSK LTVDKSRWQQ |
| 421 | GNVFSCSVMH EALHNHYTQK SLSLSPGK* |

As used herein gevokizumab, which is defined under INN number 9310, has the following sequence

| Heavy chain / Chaîne lourde / Cadena pesada | |
|---|---|
| (SEQ ID NO: 1009) | |
| QVQLQESGPG LVKPSQTLSL TCSFSGFSLS TSGMGVGWIR QPSGKGLEWL | 50 |
| AHIWWDGDES YNPSLKSRLT ISKDTSKNQV SLKITSVTAA DTAVYFCARN | 100 |
| RYDPPWFVDW GQGTLVTVSS ASTKGPSVFP LAPCSRSTSE STAALGCLVK | 150 |
| DYFPEPVTVS WNSGALTSGV HTFPAVLQSS GLYSLSSVVT VTSSNFGTQT | 200 |
| YTCNVDHKPS NTKVDKTVER KCCVECPPCP APPVAGPSVF LFPPKPKDTL | 250 |
| MISRTPEVTC VVVDVSHEDP EVQFNWYVDG MEVHNAKTKP REEQFNSTFR | 300 |
| VVSVLTVVHQ DWLNGKEYKC KVSNKGLPAP IEKTISKTKG QPREPQVYTL | 350 |
| PPSREEMTKN QVSLTCLVKG FYPSDIAVEW ESNGQPENNY KTTPPMLDSD | 400 |
| GSFFLYSKLT VDKSRWQQGN VFSCSVMHEA LHNHYTQKSL SLSPG | 445 |
| Light chain / Chaîne légère / Cadena ligera | |
| (SEQ ID NO: 1010) | |
| DIQMTQSTSS LSASVGDRVT ITCRASQDIS NYLSWYQQKP GKAVKLLIYY | 50 |
| TSKLHSGVPS RFSGSGSGTD YTLTISSLQQ EDFATYFCLQ GKMLPWTFGQ | 100 |
| GTKLEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSFN RGEC | 214 |

By “IL-113 binding antibody” is meant any antibody capable of binding to the IL-1β specifically and consequently inhibiting or modulating the binding of IL-1β to its receptor and further consequently inhibiting IL-1β function.

As used herein, the term “functional fragment” of an antibody as used herein, refers to portions or fragments of an antibody that retain the ability to specifically bind to an antigen (e.g., IL-1β). Examples of binding fragments encompassed within the term “functional fragment” of an antibody include single chain Fv (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989), which consists of a VH domain; and an isolated complementarity determining region (CDR).

Other features, objects, and advantages of the invention will be apparent from the description and drawings, and from the claims.

The following Examples illustrate the invention described above; they are not, however, intended to limit the scope of the invention in any way.

### EXAMPLE

The Example below is set forth to aid in the understanding of the invention but is not intended, and should not be construed, to limit its scope in any way.

### Example 1

A Phase III, Multicenter, Randomized, Double Blind, Placebo-Controlled Study Evaluating the Efficacy and Safety of Canakinumab Versus Placebo as Adjuvant Therapy in Adult Subjects with Stages II-IIIA and IIIB (T>5 cm N2) Completely Resected (R0) Non-Small Cell Lung Cancer (NSCLC)

The purpose of this prospective, multicenter, randomized, double blind, placebo-controlled phase III study is to evaluate the efficacy and safety of canakinumab as adjuvant therapy, following standard of care for completely resected (R0) AJCC/UICC v. 8 stages II-IIIA and stage IIIB (T>5 cm N2) NSCLC subjects.

### Study Design

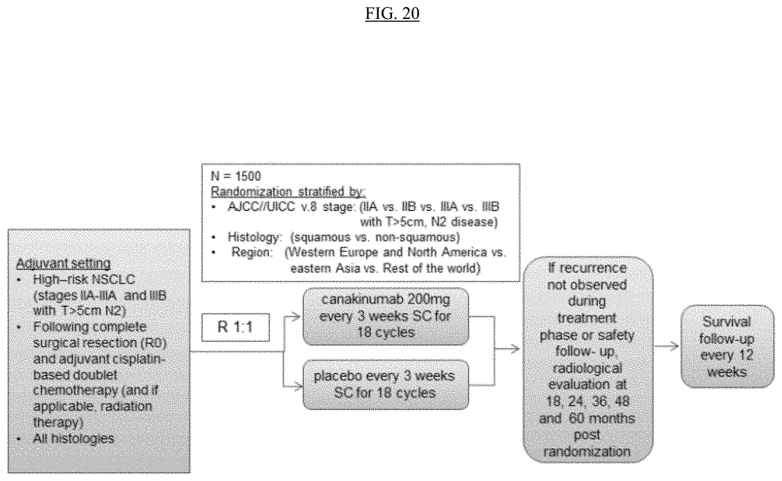

This phase III study CACZ885T2301 (FIG. 20) will enroll adult subjects with completely resected (R0) NSCLC AJCC/UICC v. 8 stages II-IIIA and IIIB (T>5 cm and N2) disease. Subjects will complete standard of care adjuvant treatments for their NSCLC, including cisplatin-based chemotherapy and mediastinal radiation therapy (if applicable), before being screened or randomized for this study. Subjects may be screened after undergoing complete surgical resection of their NSCLC and having R0 status confirmed (negative margins on pathologic review), after completing adjuvant cisplatin-based doublet chemotherapy if applicable, (and, if applicable, radiation therapy for stage IIIA N2 or IIIB N2 disease) and after all entry criteria are met. Subjects must not have had preoperative neo-adjuvant chemotherapy or radiotherapy to achieve the R0 status. Approximately 1500 subjects will be randomized 1:1 to canakinumab or matching placebo.

### Dosing Regimen

The study is double-blind. All eligible subjects will be randomized to one of the following two treatment arms in a 1:1 ratio:

Canakinumab 200 mg s.c. on day 1 of every 21-day cycle for 18 cycles

Placebo s.c. on day 1 of every 21-day cycle for 18 cycles

Randomization will be stratified by AJCC/UICC v. 8 stage: IIA versus IIB versus IIIA versus IIIB with T>5 cm, N2 disease; Histology: squamous versus non-squamous; and Region: Western Europe and North America vs. eastern Asia vs. Rest of the world (RoW). Subjects will continue their assigned treatment until they complete 18 cycles or experience any one of the following: disease recurrence as determined by Investigator, unacceptable toxicity that precludes further treatment, treatment discontinuation at the discretion of the Investigator or subject, or death, or lost to follow-up, whichever occurs first. It is postulated that the one year duration of adjuvant treatment will provide an acceptable benefit in subjects who have intermediate or high risk of developing disease recurrence. If disease recurrence is not observed during the treatment phase, subjects will be followed until disease recurrence, withdrawal of consent by the subject, subject is lost to follow up, death or the sponsor terminates the study for up to five years. All subjects who discontinue from the study treatment will be followed up every 12 weeks for survival until the final overall survival (OS) analysis or death, lost to follow-up or withdrawal of consent for survival follow-up.

Standard of care includes complete resection of the NSCLC with margins free of cancer. Four cycles of cisplatin-based doublet chemotherapy are required for all stage IIB-IIIA and IIIB (T>5 cm N2) disease subjects (except if not tolerated, in which case at least 2 cycles of adjuvant chemotherapy are required); chemotherapy is recommended but not mandatory for stage IIA with T (>4-5 cm). Radiation therapy to mediastinal nodes is suggested but not required for all stage IIIA N2 and IIIB (T>5 cm N2) disease subjects. All subjects must have had complete surgical resection of their NSCLC to be eligible for study entry; and margins must be pathologically reviewed and documented as negative. Comparisons will be made between the arms for efficacy: DFS, OS, LCSS and Quality of Life measures (EQ-5D-5L and EORTC QLQ-C30/LC13) and for safety.

Detection of first disease recurrence will be done by clinical evaluation that includes physical examination, and radiological tumor measurements as determined by the investigator. In case of non-conclusive radiological evidence, a biopsy should be performed to confirm recurrence. The following assessments are required at screening/baseline: Chest, abdomen and pelvis CT or MRI, brain MRI and whole body bone scan, if clinically indicated. Subsequent imaging assessments will be done every 12 weeks (±7 days) for the first year (treatment phase) following Cycle 1 Day 1, then every 26 weeks during years two and three, and annually during years four and five (post-treatment surveillance phase). The intervals between imaging assessments across all study phases should be respected as described above regardless of whether study treatment is temporarily withheld or permanently discontinued before the last scheduled dose administration on Cycle 18 Day 1, or if unscheduled assessments are performed. If a subject discontinues study treatment for reasons other than recurrence, recurrence assessments should continue as per the scheduled visits until disease recurrence, withdrawal of consent by the subject, subject is lost to follow up, death, or the sponsor terminates the study.

### Primary Objective and Key Secondary Objective:

### Primary Objective

The primary objective is to compare the Disease-free survival (DFS) in the canakinumab versus placebo arms as determined by local investigator assessment.

### Statistical Hypothesis, Model, and Method of Analysis

Assuming proportional hazards model for DFS, the following statistical hypotheses will be tested to address the primary efficacy objective:

H01 (null hypotheses): Θ1≥0 vs. Ha1 (alternative hypotheses): Θ1<0

Where Θ1 is the log hazard ratio of DFS in the canakinumab (investigational) arm vs. placebo (control) arm.

The primary efficacy analysis to test this hypothesis and compare the two treatment groups will consist of a stratified log-rank test at an overall one-sided 2.5% level of significance. The stratification will be based on the following randomization stratification factors: AJCC/UICC v. 8 stage IIA versus IIB versus IIIA versus IIIB with T>5 cm N2 disease; Histology: squamous versus non-squamous; and Region: Western Europe and North America vs. eastern Asia vs. Rest of the world (RoW). The hazard ratio for DFS will be calculated, along with its 95% confidence interval, from a stratified Cox model using the same stratification factors as for the log-rank test.

### Key Secondary Objective

The key secondary objective is to determine whether treatment with canakinumab prolongs overall survival OS compared with placebo arm. OS is defined as the time from the date of randomization to the date of death due to any cause. If a subject is not known to have died, then OS will be censored at the latest date the subject was known to be alive (on or before the cut-off date). Assuming proportional hazards model for OS, the following statistical hypotheses will be tested only if DFS is statistically significant:

H02 (null hypotheses): Θ2≥0 vs. Ha2 (alternative hypotheses): Θ2<0

Where Θ2 is the log hazard ratio of OS in the canakinumab (investigational) arm vs. placebo (control) arm. The analysis to test these hypotheses will consist of a stratified log-rank test at an overall one-sided 2.5% level of significance. The stratification will be based on the following randomization stratification factors: AJCC/UICC v. 8 stage IIA versus IIB versus IIIA versus IIIB T>5 cm N2 disease; Histology: squamous versus non-squamous; and Region: Western Europe and North America vs. eastern Asia vs. Rest of the world (RoW).

The OS distribution will be estimated using the Kaplan-Meier method, and Kaplan-Meier curves, medians and 95% confidence intervals of the medians will be presented for each treatment group. The hazard ratio for OS will be calculated, along with its 95% confidence interval, using a stratified Cox model.

### Secondary Objectives

1. To compare lung cancer specific survival in the canakinumab arm versus placebo arm: Lung cancer specific survival (LCSS) is defined as the time from the date of randomization to the date of death due to lung cancer. Analyses will be based on the FAS population according to the randomized treatment group and strata assigned at randomization. The LCSS distribution will be estimated using the Kaplan-Meier method, and Kaplan-Meier curves, medians and 95% confidence intervals of the medians will be presented for each treatment group. The hazard ratio for LCSS will be calculated, along with its 95% confidence interval, using a stratified Cox model.
2. To characterize the safety profile of canakinumab

Frequency of AEs, ECGs and Laboratory Abnormalities

3. To characterize the pharmacokinetics of canakinumab therapy

Serum concentration-time profiles of canakinumab and appropriate individual PK parameters based on population PK model

4. To characterize the prevalence and incidence of immunogenicity (antidrug antibodies, ADA) of canakinumab

Serum Concentrations of Anti-Canakinumab Antibodies

5. To assess the effect of canakinumab versus placebo on PROs (EORTC QLQ-C30 with QLQ-LC13 incorporated and EQ-5D) including functioning and health-related quality of life Time to definitive 10 point deterioration symptom scores of pain, cough and dyspnea per QLQ-LC13 questionnaire are primary PRO variables of interest. Time to definitive deterioration in global health status/QoL, shortness of breath and pain per QLQ-C30 together with the utilities derived from EQ-5D-5L are secondary PRO variables of interest The European Organization for Research and Treatment of Cancer's core quality of life questionnaire EORTC-QLQC30 (version 3.0) and it's lung cancer specific module QLQLC13 (version 1.0) will be used to collect data on the subject's functioning, disease-related symptoms, health-related quality of life, and health status. The EQ-5D-5L will be used for the purpose of the computation of utilities that can be used in health economic studies. The EORTC QLQ-C30/LC13 as well as the EQ-5D-5L are reliable and valid measures frequently used in clinical trials of subjects with lung cancer and previously used in the adjuvant setting (Bezjak et al 2008).

### Example 2A

### Blocking IL-1β Signaling Alters Blood Vessels in the Bone Microenvironment

Background: We have recently identified interleukin-1β (IL-1β) as a potential biomarker for predicting breast cancer patients at increased risk for developing bone metastasis. In addition we have shown that blocking IL-1β activity inhibits development of bone metastases from breast cancer cells disseminated in bone and reduces tumour angiogenesis. We hypothesise that interactions between IL-1β and IL-1R also promotes formation of new blood vessels in the bone microenvironment stimulating development of metastases at this site.

Objectives: Investigate the effects of blocking IL-1β activity on blood vessel formation within bone.

Methodology: The effects of IL-1R inhibition on vasculature in trabecular bone were determined in mice treated with 1 mg/kg of the IL-1R antagonist (anakinra) for 21/31 days, the IL-1β antibody canakinumab (Ilaris) for 0-96 hours or in genetically engineered IL-1R1 knockout (KO) mice. Vasculature was visualised following CD34 and endomucin immunohistochemistry and the concentration of vascular endothelial growth factor (VEGF) and endothelin-1 in serum and/or bone marrow was determined by ELISA. Effects on bone volume were measured by Micro computed tomography (uCT).

Results: Canakinumab (Ilaris) caused a significant decrease in the length of new blood vessels from 0.09 mm (control) to 0.06 mm (24 hours Ilaris) (P=0.0319). IL-1R1 KO mice and mice treated with anakinra demonstrated a downwards trend in the average length of new blood vessels. Inhibition of IL-1R resulted in increased trabecular bone volume. Anakinra caused a 69% decrease in the concentration of endothelin-1 in mice treated for 31 days (P=0.0269) and a 22% decrease in VEGF concentration in mice treated for 21 days (P=0.0104). Canakinumab (Ilaris) caused a 46% reduction in VEGF concentration and a 47% reduction in endothelin-1 concentration in mice treated for 96 hours.

Conclusions: These data demonstrate that IL-1R activity plays an important role in the formation of new vasculature in bone and inhibiting its activity pharmacologically has potential as a novel treatment for breast cancer bone metastasis.

### Example 2B

### IL-1B Signalling Regulates Breast Cancer Bone Metastasis

Breast cancer bone metastases is incurable and associates with poor prognosis in patients. After homing and colonising the bone, breast cancer cells remain dormant, until signals from the microenvironment stimulate proliferation of these disseminated cells to form overt metastases. We have recently identified interleukin 1B (IL-1B) as a potential marker for predicting breast cancer patients at increased risk for developing metastasis and established a role for IL-1 signalling in tumour cell dormancy in bone. We hypothesise that tumour derived and microenvironment dependent IL-1B play major roles in breast cancer metastasis and growth in bone.

Here, we report our findings on the role of IL-1B signalling in breast cancer bone metastasis: Using a murine model of spontaneous human breast cancer metastasis to human bone, we found that administration of the clinically available anti-IL-1B monoclonal antibody, Ilaris, significantly reduced bone metastasis, while increasing primary tumour growth. Whereas, blockade of IL1R1 using a recombinant form of the receptor antagonist, Anakinra, delayed onset of breast cancer metastasis in human bone, without affecting the development of primary breast cancer. These finding suggest that IL1 signalling might exert different functions in breast cancer progression at the primary and metastatic site. Our data further highlight roles for both tumour derived and microenvironment derived IL-1 signalling in tumour cell dissemination and growth in bone: Inhibition of IL-1B/IL-1R1 with Ilaris or Anakinra reduced bone turnover and neovascularisation rendering the bone microenvironment less permissive for growth of breast cancer cells. In addition, overexpression of IL1B or IL1R in human breast cancer cells increased bone metastases from tumour cells injected directly into the circulation in vivo. These data demonstrate that IL-1B/IL-1R1 signalling plays an important role in the formation of bone metastasis and inhibiting its activity pharmacologically has potential as a novel treatment for breast cancer bone metastasis.

### Example 2C

### Targeting IL1b-Wnt Signalling Prevents Breast Cancer Colonisation in the Bone Microenvironment

Dissemination of tumour cells to bone marrow is an early event in breast cancer, however these cells may lie dormant in the bone environment for many years prior to eventual colonisation. Treatment for bone metastases is not curative, therefore new adjuvant therapies preventing disseminated cells from becoming metastatic lesions may be an effective therapeutic option to improve clinical outcomes. There is evidence that cancer stemcells (CSCs) within breast tumours are the cells capable of metastasis; however, little is known about which bone marrow-derived factors support dormant CSC survival and eventual colonisation. Using in vitro culture of primary human bone marrow and patient-derived breast cancer cells, and in vivo metastasis models of human breast cancer cells implanted into mice, we investigated signalling pathways regulating CSC colony formation in bone.

We demonstrate that exposure to the bone microenvironment stimulates breast CSC colony formation in 15/17 patient-derived early breast cancers in vitro, and promotes a 3-4-fold increase in colony formation in breast cancer cells injected intra-femorally in vivo (p\0.05). Further, we establish that ILlb secreted by human bone marrow induces breast CSC colony formation via intracellular NFkB signalling that induces Wnt secretion. Crucially, we show that inhibiting either IL1b (using an IL1b neutralising antibody or the IL1R antagonist Anakinra) or Wnt signalling (using Vantictumab, a therapeutic antibody which binds 5/10 Frizzled receptors), reverses induction of CSC activity by the bone marrow in vitro (Anakinra; p\0.0001, Vantictumab; p\0.01) and prevents spontaneous bone metastasis in vivo (IL1b neutralising antibody; p\0.02, Vantictumab; p\0.01). These data indicate that IL-ab-Wnt inhibitors will prevent disseminated CSCs from forming metastatic colonies in bone, and represent an attractive adjuvant therapeutic opportunity in breast cancer. Drugs which target IL-1b (Anakinra and Canakinumab) are FDA-approved for other indications, and anti-Wnt treatments (Vantictumab) are in clinical trials in cancer, making this a viable therapeutic target in breast cancer patients.

### Example 2C

### Targeting IL-1β-Wnt Signalling to Prevent Breast Cancer Colonisation in the Bone Microenvironment

Dissemination of tumour cells to bone marrow is an early event in breast cancer, but these cells may lie dormant in the bone environment for many years before the development of clinical bone metastases. There is evidence that cancer stem cells (CSCs) within breast tumours are the cells capable of metastasis, but the effect of the bone environment on the regulation of CSCs has not been investigated. We used two models to study this: in vitro culture of primary human bone marrow and patient-derived breast cancer cells, and in vivo intra-femoral injections of luciferase/tdTomato-labelled breast cancer cells into immune-deficient mice. CSC activity following isolation from the bone environment was measured using mammosphere colony formation.

We demonstrate that exposure to the bone microenvironment stimulates breast CSC colony formation in 15/17 patient-derived early breast cancers in vitro, and promotes a 3-4-fold increase in colony formation in breast cancer cells injected into the femoral bone marrow of mice in vivo (p<0.05). Furthermore, we establish that IL1b secreted by human bone marrow induces breast CSC colony formation via an induction of Wnt signalling in breast cancer cells. We show that inhibiting IL1β (using an IL1β neutralising antibody or the IL1R antagonist Anakinra) or Wnt signalling (using Vantictumab, a therapeutic antibody which binds 5/10 Frizzled receptors), reverses induction of CSC activity by the bone marrow in vitro (Anakinra; p<0.0001, Vantictumab; p<0.01), and prevents spontaneous bone metastasis in vivo (IL1β neutralising antibody; p<0.02, Vantictumab; p<0.01).

These data indicate that IL-1β-Wnt inhibitors may prevent disseminated CSCs from forming metastatic colonies in the bone, and should be considered as an adjuvant therapeutic opportunity in breast cancer. Clinically available drugs against IL-1β (Anakinra and Canakinumab) are licensed for other applications, and anti-Wnt treatments (Vantictumab) are in clinical trials, making this pathway a viable therapeutic target in breast cancer patients.

### Example 2D

### Anti-IL1B Therapy and Standard of Care Agents: A Double Edged-Sword to Halt Breast Cancer Bone Metastasis

Breast cancer bone metastases is incurable and associates with poor prognosis in patients. After homing and colonising the bone, breast cancer cells remain dormant, until signals from the microenvironment stimulate proliferation of these disseminated cells to form overt metastases. We have recently identified interleukin 1B (IL-1B) as a potential marker for predicting breast cancer patients at increased risk for developing metastasis and established a role for IL-1 signalling in tumour cell dormancy in bone. We hypothesise that tumour-derived and microenvironment-dependent IL-1B play major roles in breast cancer metastasis and growth in bone.

Here, we report our findings on the role of IL-1B signalling in breast cancer bone metastasis. Using a murine model of spontaneous human breast cancer metastasis to human bone, we found that administration of the clinically available anti-IL-1B monoclonal antibody, Ilaris, or the clinically available recombinant form of the receptor antagonist, Anakinra, reduced bone metastasis (photons/sec mean values: 3.60E+06 Placebo, 4.83E+04 Anakinra, 6.01E+04 Ilaris). In line with this finding, IL-1B or IL-1R1 overexpression in human breast cancer cells resulted in enhanced tumour cell dissemination and growth in bone (12.5, 75 and 50% animals with tumour in bone in control, IL-1B and IL-1R-overexpressing cells, respectively). The use of standard of care agents and/or anti-resorptive drugs is a treatment strategy for patients affected by breast cancer. Here, we combine anti-IL1B treatment (Anakinra) with standard of care agent (Doxorubicin) and/or anti-resorptive agent (Zoledronic acid) in a syngeneic model of breast cancer metastasis. Our experiments show that the triple treatment significantly impairs breast cancer metastasis (p=0.0084).

In conclusion, these data demonstrate that IL-1B/IL-1R1 signalling plays an important role in the formation of bone metastasis and inhibiting its activity pharmacologically alone or in combination with standard of care therapies has potential as a novel treatment for bone metastasis.

### Example 3

### Tumor-derived IL-1β Induces Differential Tumor Promoting Mechanisms in Metastasis

### Materials and Methods

### Cell Culture

Human breast cancer MDA-MB-231-Luc2-TdTomato (Calliper Life Sciences, Manchester UK), MDA-MB-231 (parental) MCF7, T47D (European Collection of Authenticated Cell Cultures (ECACC)), MDA-MB-231-IV (Nutter et al., 2014) as well as bone marrow HS5 (ECACC) and human primary osteoblasts OB1 were cultured in DMEM+10% FCS (Gibco, Invitrogen, Paisley, UK). All cell lines were cultured in a humidified incubator under 5% CO2 and used at low passage>20.

### Transfection of Tumor Cells:

Human MDA-MB-231, MCF 7 and T47D cells were stably transfected to overexpress genes IL1B or IL1R1 using plasmid DNA purified from competent E. coli that have been transduced with an ORF plasmid containing human IL1B or IL1R1 (Accession numbers NM_000576 and NM_0008777.2, respectively) with a C-terminal GFP tag (OriGene Technologies Inc. Rockville Md.). Plasmid DNA purification was performed using a PureLink™ HiPure Plasmid Miniprep Kit (ThemoFisher) and DNA quantified by UV spectroscopy before being introduced into human cells with the aid of Lipofectamine II (ThermoFisher). Control cells were transfected with DNA isolated from the same plasmid without IL-1B or IL-1R1 encoding sequences.

### In Vitro Studies

In vitro studies were carried out with and without addition of 0-5 ng/ml recombinant IL-1β (R&D systems, Wiesbaden, Germany)+/−50 μM IL-1Ra (Amgen, Cambridge, UK). Cells were transferred into fresh media with 10% or 1% FCS. Cell proliferation was monitored every 24 h for up to 120 h by manual cell counting using a 1/400 mm² hemocytometer (Hawkley, Lancing UK) or over a 72 h period using an Xcelligence RTCA DP Instrument (Acea Biosciences, Inc). Tumor cell invasion was assessed using 6 mm transwell plates with an 8 μm pore size (Corning Inc) with or without basement membrane (20% Matrigel; Invitrogen). Tumor cells were seeded into the inner chamber at a density of 2.5×10⁵ for parental as well as MDA-MB-231 derivatives and 5×10⁵ for T47D in DMEM+1% FCS and 5×10⁵ OB1 osteoblast cells supplemented with 5% FCS were added to the outer chamber. Cells were removed from the top surface of the membrane 24 h and 48 h after seeding and cells that had invaded through the pores were stained with hematoxylin and eosin (H&E) before being imaged on a Leica DM7900 light microscope and manually counted.

Migration of cells was investigated by analyzing wound closure: Cells were seeded onto 0.2% gelatine in 6-well tissue culture plates (Costar; Corning, Inc) and, once confluent, 10 μg/ml mitomycin C was added to inhibit cell proliferation and a 50 μm scratch made across the monolayer. The percentage of wound closure was measured at 24 h and 48 h using a CTR7000 inverted microscope and LAS-AF v2.1.1 software (Leica Applications Suite; Leica Microsystems, Wetzlar, Germany). All proliferation, invasion and migration experiments were repeated using the Xcelligence RTCA DP instrument and RCTA Software (Acea Biosystems, Inc).

For co-culture studies with human bone 5×10⁵ MDA-MB-231 or T47D cells were seeded onto tissue culture plastic or into 0.5 cm³ human bone discs for 24 h. Media was removed and analysed for concentration of IL-1β by ELISA. For co-culture with HS5 or OB1 cells, 1×10⁵ MDA-MB-231 or T47D cells were cultured onto plastic along with 2×10⁵ HS5 or OB1 cells. Cells were sorted by FACS 24 h later and counted and lysed for analysis of IL-1β concentration. Cells were collected, sorted and counted every 24 h for 120 h.

### Animals

Experiments using human bone grafts were carried out in 10-week old female NOD SCID mice. In IL-1β/IL-1R1 overexpression bone homing experiments 6 to 8-week old female BALB/c nude mice were used. To investigate effects of IL-1β on the bone microenvironment 10-week old female C57BL/6 mice (Charles River, Kent, UK) or IL-1R1^{-/-} mice (Abdulaal et al., 2016) were used. Mice were maintained on a 12 h:12 h light/dark cycle with free access to food and water. Experiments were carried out with UK home office approval under project licence 40/3531, University of Sheffield, UK.

### Patient Consent and Preparation of Bone Discs

All patients provided written, informed consent prior to participation in this study. Human bone samples were collected under HTA licence 12182, Sheffield Musculoskeletal Biobank, University of Sheffield, UK. Trabecular bone cores were prepared from the femoral heads of female patients undergoing hip replacement surgery using an Isomat 4000 Precision saw (Buehler) with Precision diamond wafering blade (Buehler). 5 mm diameter discs were subsequently cut using a bone trephine before storing in sterile PBS at ambient temperature.

### In Vivo Studies

To model human breast cancer metastasis to human bone implants two human bone discs were implanted subcutaneously into 10-week old female NOD SCID mice (n=10/group) under isofluorane anaesthetic. Mice received an injection of 0.003 mg vetergesic and Septrin was added to the drinking water for 1 week following bone implantation. Mice were left for 4 weeks before injecting 1×10⁵MDA-MB-231 Luc2-TdTomato, MCF7 Luc2 or T47D Luc2 cells in 20% Martigel/79% PBS/1% toluene blue into the two hind mammary fat pads. Primary tumor growth and development of metastases was monitored weekly using an IVIS (Luminol) system (Caliper Life Sciences) following sub-cutaneous injection of 30 mg/ml D-luciferin (Invitrogen). On termination of experiments mammary tumors, circulating tumor cells, serum and bone metastases were resected. RNA was processed for downstream analysis by real time PCR, and cell lysates were taken for protein analysis and whole tissue for histology as previously described (Nutter et al., 2014; Ottewell et al., 2014a).

For therapeutic studies in NOD SCID mice, placebo (control), 1 mg/kg IL-1Ra (Anakinra®) daily or 10 mg/kg canakinumab subcutaneously every 14 days were administered starting 7 days after injection of tumor cells. In BALB/c mice and C57BL/6 mice 1 mg/kg IL-1Ra was administered daily for 21 or 31 days or 10 mg/kg canakinumab was administered as a single subcutaneous injection. Tumor cells, serum, and bone were subsequently resected for downstream analysis.

Bone metastases were investigated following injection of 5×10⁵ MDA-MB-231 GFP (control), MDA-MB-231-IV, MDA-MB-231-IL-1B-positive or MDA-MB-231-IL-1R1-positive cells into the lateral tail vein of 6 to 8-week old female BALB/c nude mice (n=12/group). Tumor growth in bones and lungs was monitored weekly by GFP imaging in live animals. Mice were culled 28 days after tumor cell injection at which timepoint hind limbs, lungs and serum were resected and processed for microcomputed tomography imaging (μCT), histology and ELISA analysis of bone turnover markers and circulating cytokines as described (Holen et al., 2016).

### Isolation of Circulating Tumor Cells

Whole blood was centrifuged at 10,000 g for 5 minutes and the serum removed for ELISA assays. The cell pellet was re-suspended in 5 ml of FSM lysis solution (Sigma-Aldrich, Pool, UK) to lyse red blood cells. Remaining cells were re-pelleted, washed 3× in PBS and re-suspended in a solution of PBS/10% FCS. Samples from 10 mice per group were pooled prior to isolation of TdTomato positive tumor cells using a MoFlow High performance cell sorter (Beckman Coulter, Cambridge UK) with the 470 nM laser line from a Coherent I-90C tenable argon ion (Coherent, Santa Clara, Calif.). TdTomato fluorescence was detected by a 555LP dichroic long pass and a 580/30 nm band pass filter. Acquisition and analysis of cells was performed using Summit 4.3 software. Following sorting cells were immediately placed in RNA protect cell reagent (Ambion, Paisley, Renfrew, UK) and stored at −80° C. before RNA extraction.

### Microcomputed Tomography Imaging:

Microcomputed tomography (μCT) analysis was carried out using a Skyscan 1172 x-ray-computed μCT scanner (Skyscan, Aartselar, Belgium) equipped with an x-ray tube (voltage, 49 kV; current, 200 uA) and a 0.5-mm aluminium filter. Pixel size was set to 5.86 μm and scanning initiated from the top of the proximal tibia as previously described (Ottewell et al., 2008a; Ottewell et al., 2008b).

### Bone Histology and Measurement of Tumor Volume:

Bone tumor areas were measured on three non-serial, H&E stained, 5 μm histological sections of decalcified tibiae per mouse using a Leica RMRB upright microscope and Osteomeasure software (Osteometrics, Inc. Decauter, USA) and a computerised image analysis system as previously described (Ottewell et al., 2008a).

### Western Blotting:

Protein was extracted using a mammalian cell lysis kit (Sigma-Aldrich, Poole, UK). 30 of protein was run on 4-15% precast polyacrylamide gels (BioRad, Watford, UK) and transferred onto an Immobilon nitrocellulose membrane (Millipore). Non-specific binding was blocked with 1% casein (Vector Laboratories) before incubation with rabbit monoclonal antibodies to human N-cadherin (D4R1H) at a dilution of 1:1000, E-cadherin (24E10) at a dilution of 1:500 or gamma-catenin (2303) at a dilution of 1:500 (Cell signalling) or mouse monoclonal GAPDH (ab8245) at a dilution of 1:1000 (AbCam, Cambridge UK) for 16 h at 4° C. Secondary antibodies were anti-rabbit or anti-mouse horse radish peroxidase (HRP; 1:15,000) and HRP was detected with the Supersignal chemiluminescence detection kit (Pierce). Band quantification was carried out using Quantity Once software (BioRad) and normalised to GAPDH.

### Gene Analysis

Total RNA was extracted using an RNeasy kit (Qiagen) and reverse transcribed into cDNA using Superscript III (Invitrogen AB). Relative mRNA expression of IL-1B (Hs02786624), IL-1R1 (Hs00174097), CASP (Caspase 1) (Hs00354836), IL1RN (Hs00893626), JUP (junction plakoglobin/gamma-catenin) (Hs00984034), N-cadherin (Hs01566408) and E-cadherin (Hs1013933) were compared with the housekeeping gene glyceraldehyde-3-phosphate dehydrogenase (GAPDH; Hs02786624) and assessed using an ABI 7900 PCR System (Perkin Elmer, Foster City, Calif.) and Taqman universal master mix (Thermofisher, UK). Fold change in gene expression between treatment groups was analysed by inserting CT values into Data Assist V3.01 software (Applied Biosystems) and changes in gene expression were only analysed for genes with a CT value of ≤25.

Assessment of IL-1β and IL-1R1 in Tumors from Breast Cancer Patients

IL-1β and IL-1R1 expression was assessed on tissue microarrays (TMA) containing primary breast tumor cores taken from 1,300 patients included in the clinical trial, AZURE (Coleman et al. 2011). Samples were taken pre-treatment from patients with stage II and III breast cancer without evidence of metastasis. Patients were subsequently randomized to standard adjuvant therapy with or without the addition of zoledronic acid for 10 years (Coleman et al 2011). The TMAs were stained for IL-1β (ab2105, 1:200 dilution, Abcam) and IL-1R1 (ab59995, 1:25 dilution, Abcam) and scored blindly under the guidance of a histopathologist for IL-1β/IL-1R1 in the tumor cells or in the associated stroma. Tumor or stromal IL-1β or IL-1R1 was then linked to disease recurrence (any site) or disease recurrence specifically in bone (+/− other sites).

### The IL-1β Pathway is Upregulated During the Process of Human Breast Cancer Metastasis to Human Bone.

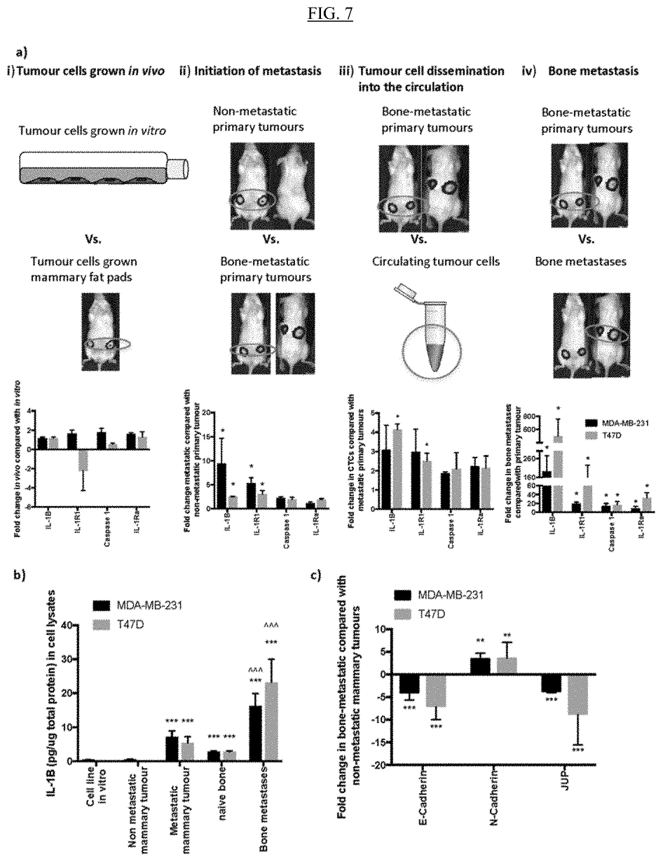

A mouse model of spontaneous human breast cancer metastasis to human bone implants was utilised to investigate how the IL-1β pathway changes through the different stages of metastasis. Using this model, the expression levels of genes associated with the IL-1β pathway increased in a stepwise manner at each stage of the metastatic process in both triple negative (MDA-MB-231) and estrogen receptor positive (ER+ve) (T47D) breast cancer cells: Genes associated with the IL-1β signalling pathway (IL-1B, IL-1R1, CASP (Caspase 1) and IL-1Ra) were expressed at very low levels in both MDA-MB-231 and T47D cells grown in vitro and expression of these genes were not altered in primary mammary tumors from the same cells that did not metastasize in vivo (FIG. 7, panel a).

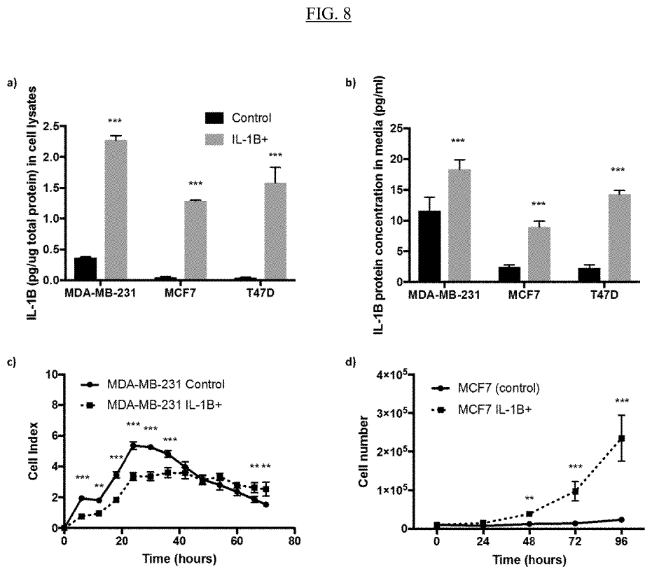

IL-1B, IL-1R1 and CASP were all significantly increased in mammary tumors that subsequently metastasized to human bone compared with those that did not metastasize (p<0.01 for both cell lines), leading to activation of IL-1β signalling as shown by ELISA for the active 17 kD IL-1β (FIG. 7, panel b; FIG. 8). IL-1B gene expression increased in circulating tumor cells compared with metastatic mammary tumors (p<0.01 for both cell lines) and IL-1B (p<0.001), IL-1R1 (p<0.01), CASP (p<0.001) and IL-1Ra (p<0.01) were further increased in tumor cells isolated from metastases in human bone compared with their corresponding mammary tumors, leading to further activation of IL-1β protein (FIG. 7; FIG. 8). These data suggest that IL-1β signalling may promote both initiation of metastasis from the primary site as well as development of breast cancer metastases in bone.

### Tumor Derived IL-113 Promotes EMT and Breast Cancer Metastasis.

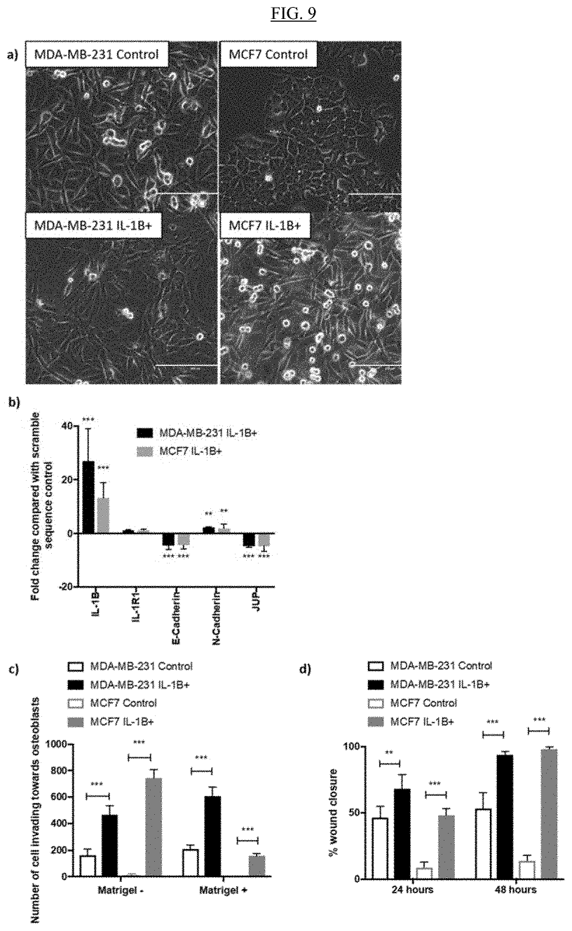

Expression levels of genes associated with tumor cell adhesion and epithelial to mesenchymal transition (EMT) were significantly altered in primary tumors that metastasised to bone compared with tumors that did not metastasise (FIG. 7, panel c). IL-1β-overexpressing cells were generated (MDA-MB-231-IL-1B+, T47D-IL-1B+ and MCF7-IL-1B+) to investigate whether tumor-derived IL-1β is responsible for inducing EMT and metastasis to bone. All IL-1β+ cell lines demonstrated increased EMT exhibiting morphological changes from an epithelial to mesenchymal phenotype (FIG. 9, panel a) as well as reduced expression of E-cadherin, and JUP (junction plakoglobin/gamma-catenin) and increased expression of N-Cadherin gene and protein (FIG. 9, panel b). Wound closure (p<0.0001 in MDA-MB-231-IL-1β+(FIG. 9, panel d); p<0.001 MCF7-IL-1β+ and T47D-IL-1β+) and migration and invasion through matrigel towards osteoblasts were increased in tumor cells with increased IL-1β signalling compared with their respective controls (MDA-MB-231-IL-1β+ (FIG. 9, panel c) p<0.0001; MCF7-IL-1β+ and T47D-IL-1β+p<0.001). Increased IL-1β production was seen in ER-positive and ER-negative breast cancer cells that spontaneously metastasized to human bone implants in vivo compared with non-metastatic breast cancer cells (FIG. 7). The same link between IL-1β and metastasis was made in primary tumor samples from patients with stage II and III breast cancer enrolled in the AZURE study (Coleman et al., 2011) that experienced cancer relapsed over a 10 year time period. IL-1β expression in primary tumors from the AZURE patients correlated with both relapse in bone and relapse at any site indicating that presence of this cytokine is likely to play a role in metastasis in general. In agreement with this, genetic manipulation of breast cancer cells to artificially overexpress IL-1β increased the migration and invasion capacities of breast cancer cells in vitro (FIG. 9).

### Inhibition of IL-1β Signaling Reduces Spontaneous Metastasis to Human Bone.

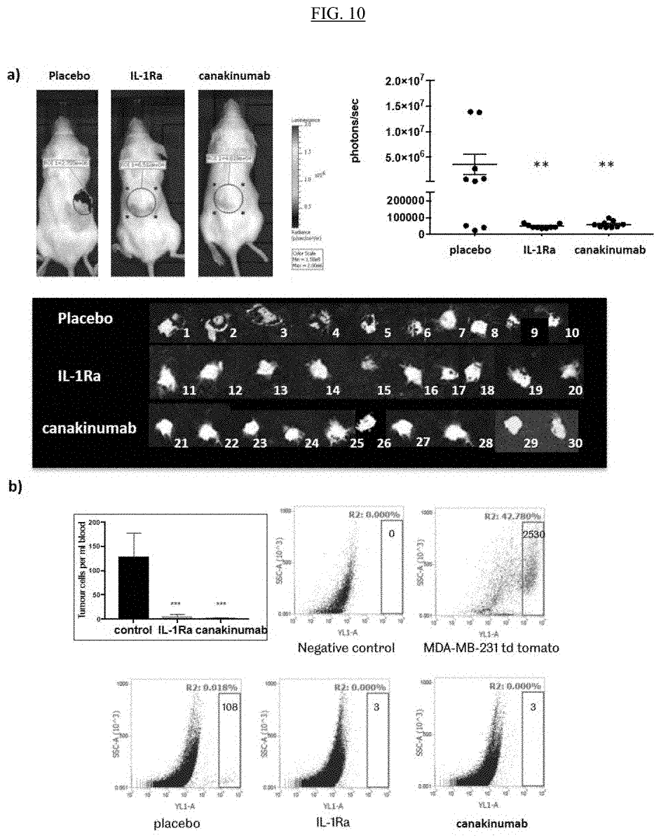

As tumor derived IL-1β appeared to be promoting onset of metastasis through induction of EMT the effects of inhibiting IL-1β signaling with IL-1Ra (Anakinra) or a human anti-IL-1β-binding antibody (canakinumab) on spontaneous metastasis to human bone implants were investigated: Both IL-1Ra and canakinumab reduced metastasis to human bone: metastasis was detected in human bone implants in 7 out of 10 control mice, but only in 4 out of 10 mice treated with IL-1Ra and 1 out of 10 mice treated with canakinumab. Bone metastases from IL-1Ra and canakinumab treatment groups were also smaller than those detected in the control group (FIG. 10, panel a). Numbers of cells detected in the circulation of mice treated with canakinumab or IL-1Ra were significantly lower than those detected in the placebo treated group: 3 and 3 tumor cells/ml were counted in whole blood from mice treated with canakinumab and anakinra, respectively, compared 108 tumor cells/ml counted in blood from placebo treated mice (FIG. 10, panel b), suggesting that inhibition of IL-1 signalling prevents tumor cells from being shed from the primary site into the circulation. Therefore, inhibition of IL-1β signaling with the anti-IL-1β antibody canakinumab or inhibition of IL-1R1 reduced the number of breast cancer cells shed into the circulation and reduced metastases in human bone implants (FIG. 10).

### Tumor Derived IL-1B Promotes Bone Homing and Colonisation of Breast Cancer Cells.

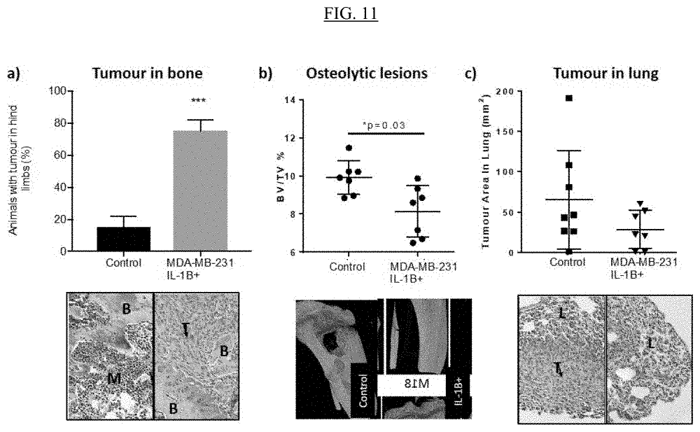

Injection of breast cancer cells into the tail vein of mice usually results in lung metastasis due to the tumor cells becoming trapped in the lung capillaries. It was previously shown that breast cancer cells that preferentially home to the bone microenvironment following intra-venous injection express high levels of IL-1β, suggesting that this cytokine may be involved in tissue specific homing of breast cancer cells to bone. In the current study, intravenous injection of MDA-MB-231-IL-1β+ cells into BALB/c nude mice resulted in significantly increased number of animals developing bone metastasis (75%) compared with control cells (12%) (p<0.001) cells (FIG. 11, panel a). MDA-MB-231-IL-1β+ tumors caused development of significantly larger osteolytic lesions in mouse bone compared with control cells (p=0.03; FIG. 11, panel b) and there was a trend towards fewer lung metastases in mice injected with MDA-MB-231-IL-1β+ cells compared with control cells (p=0.16; FIG. 11, panel c). These data suggest that endogenous IL-1β can promote tumor cell homing to the bone environment and development of metastases at this site.

### Tumor Cell-Bone Cell Interactions Further Induce IL-1B and Promote Development of Overt Metastases.

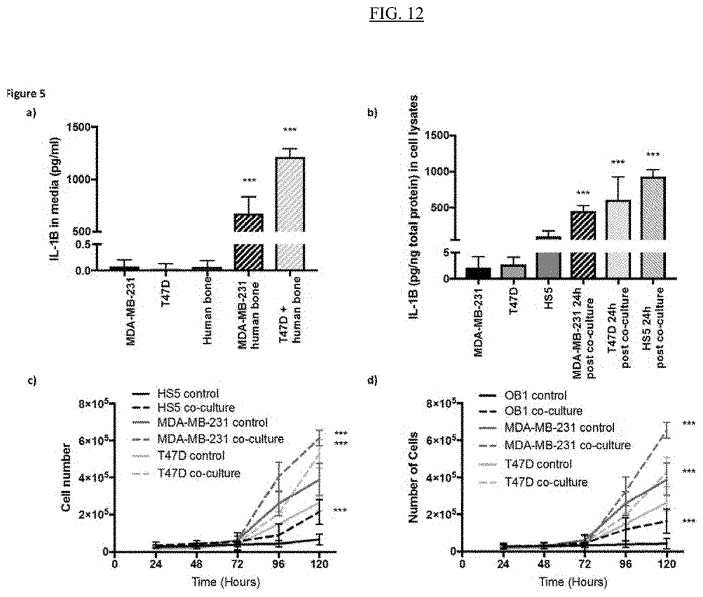

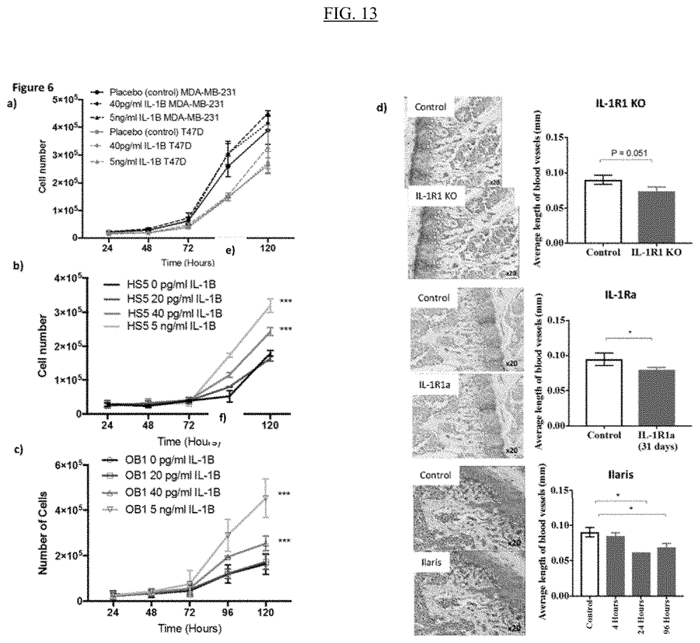

Gene analysis data from a mouse model of human breast cancer metastasis to human bone implants suggested that the IL-1β pathway was further increased when breast cancer cells are growing in the bone environment compared with metastatic cells in the primary site or in the circulation (FIG. 7, panel a). It was therefore investigated how IL-1β production changes when tumor cells come into contact with bone cells and how IL-1β alters the bone microenvironment to affect tumor growth (FIG. 12). Culture of human breast cancer cells into pieces of whole human bone for 48 h resulted in increased secretion of IL-1β into the medium (p<0.0001 for MDA-MB-231 and T47D cells; FIG. 12, panel a). Co-culture with human HS5 bone marrow cells revealed the increased IL-1β concentrations originated from both the cancer cells (p<0.001) and bone marrow cells (p<0.001), with IL-1β from tumor cells increasing ˜1000 fold and IL-1B from HS5 cells increasing ˜100 fold following co-culture (FIG. 12, panel b).

Exogenous IL-1β did not increase tumor cell proliferation, even in cells overexpressing IL-1R1. Instead, IL-1β stimulated proliferation of bone marrow cells, osteoblasts and blood vessels that in turn induced proliferation of tumor cells (FIG. 11). It is therefore likely that arrival of tumor cells expressing high concentrations of IL-1β stimulate expansion of the metastatic niche components and contact between IL-1β expressing tumor cells and osteoblasts/blood vessels drive tumor colonization of bone. The effects of exogenous IL-1β as well as IL-1β from tumor cells on proliferation of tumor cells, osteoblasts, bone marrow cells and CD34⁺ blood vessels were investigated: Co-culture of HS5 bone marrow or OB1 primary osteoblast cells with breast cancer cells caused increased proliferation of all cell types (P<0.001 for HS5, MDA-MB-231 or T47D, FIG. 12, panel c) (P<0.001 for OB1, MDA-MB-231 or T47D, FIG. 12, panel d). Direct contact between tumor cells, primary human bone samples, bone marrow cells or osteoblasts promoted release of IL-1β from both tumor and bone cells (FIG. 12). Furthermore, administration of IL-1β increased proliferation of HS5 or OB1 cells but not breast cancer cells (FIG. 13, panels a and b), suggesting that tumor cell-bone cell interactions promote production of IL-1β that can drive expansion of the niche and stimulate the formation of overt metastases.

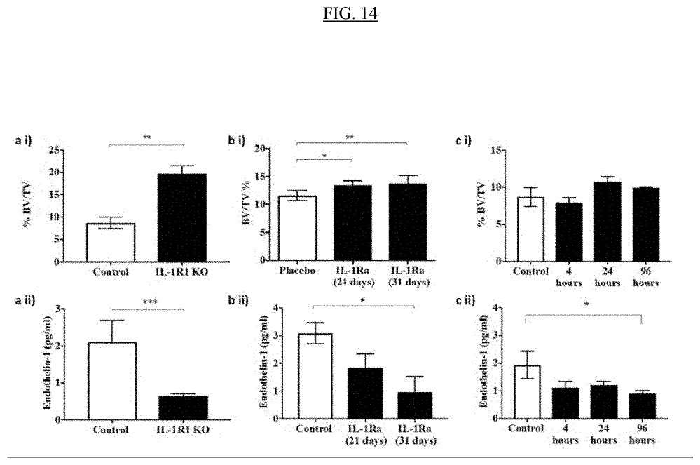

IL-1β signalling was also found to have profound effects on the bone microvasculature: Preventing IL-1β signaling in bone by knocking out IL-1R1, pharmacological blockade of IL-1R with IL-1Ra or reducing circulating concentrations of IL-1β by administering the anti-IL-1β binding antibody canakinumab reduced the average length of CD34⁺ blood vessels in trabecular bone, where tumor colonisation takes place (p<0.01 for IL-1Ra and canakinumab treated mice) (FIG. 13, panel c). These findings were confirmed by endomeucin staining which showed decreased numbers of blood vessels as well as blood vessel length in bone when IL-1β signaling was disrupted. ELISA analysis for endothelin 1 and VEGF showed reduced concentrations of both of these endothelial cell markers in the bone marrow for IL-1R1^{-/-} mice (p<0.001 endothelin 1; p<0.001 VEGF) and mice treated with IL-1R antagonist (p<0.01 endothlin 1; p<0.01 VEGF) or canakinumab (p<0.01 endothelin 1; p<0.001 VEGF) compared with control (FIG. 14). These data suggest that tumor cell-bone cell associated increases in IL-1β and high levels of IL-1β in tumor cells may also promote angiogenesis, further stimulating metastases.

### Tumor Derived IL-1β Predicts Future Breast Cancer Relapse in Bone and Other Organs in Patient Material

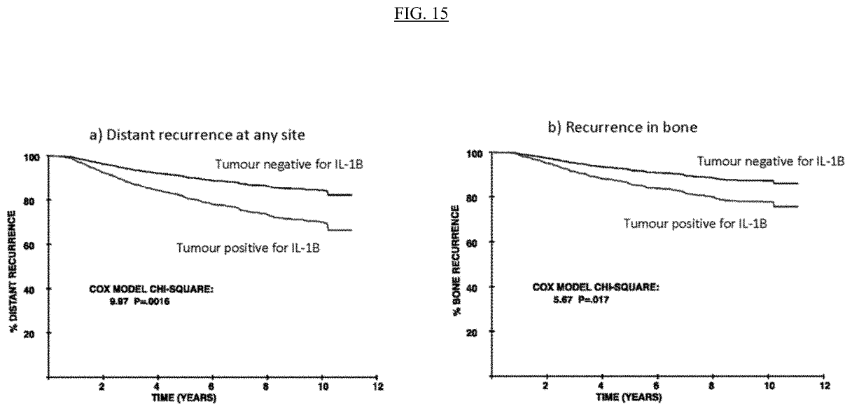

To establish the relevance of the findings in a clinical setting the correlation between IL-1β and its receptor IL-1R1 in patient samples was investigated. 1300 primary tumor samples from patients with stage II/III breast cancer with no evidence of metastasis (from the AZURE study (Coleman et al., 2011)) were stained for IL-1R1 or the active (17 kD) form of IL-1β, and biopsies were scored separately for expression of these molecules in the tumor cells and the tumor associated stroma. Patients were followed up for 10 years following biopsy and correlation between IL-1β/IL-1R1 expression and distant recurrence or relapse in bone assessed using a multivariate Cox model. IL-1β in tumor cells strongly correlated with distant recurrence at any site (p=0.0016), recurrence only in bone (p=0.017) or recurrence in bone at any time (p=0.0387) (FIG. 15). Patients who had IL-1β in their tumor cells and IL-1R1 in the tumor associated stroma were more likely to experience future relapse at a distant site (p=0.042) compared to patients who did not have IL-1β in their tumor cells, indicating that tumor derived IL-1β may not only promote metastasis directly but may also interact with IL-1R1 in the stroma to promote this process. Therefore, IL-1β is a novel biomarker that can be used to predict risk of breast cancer relapse.

### Example 4

Simulation of Canakinumab PK Profile and hsCRP Profile for Lung Cancer Patients.

A model was generated to characterize the relationship between canakinumab pharmacokinetics (PK) and hsCRP based on data from the CANTOS study.

The following methods were used in this study: Model building was performed using the first-order conditional estimation with interaction method. The model described the logarithm of the time resolved hsCRP as:

y(tᵢⱼ)=y_{0,1}+y_{eff}(tᵢⱼ)

where y_{0,1} is a steady state value and y_{eff} (tᵢⱼ) describes the effect of the treatment and depends on the systemic exposure. The treatment effect was described by an Emax-type model,

$${y}_{eff} ( {t}_{i j} ) = {E}_{max , i} \frac{c ( {t}_{i j} )}{c ( {t}_{i j} ) + IC {50}_{i}}$$

where E_{max,i} is the maximal possible response at high exposure, and IC50ᵢ is the concentration at which half maximal response is obtained.
The individual parameters, E_{max,i} and y_{0,1} and the logarithm of IC50ᵢ were estimated as a sum of a typical value, covariate effects covpar*covᵢ and normally distributed between subject variability. In the term for the covariate effect covpar refers to the covariate effect parameter being estimated and covᵢ is the value of the covariate of subject i. Covariates to be included were selected based on inspection of the eta plots versus covariates. The residual error was described as a combination of proportional and additive term.

The logarithm of baseline hsCRP was included as covariate on all three parameters (E_{max,i}, y_{0,1} and IC50ᵢ). No other covariate was included into the model. All parameters were estimated with good precision. The effect of the logarithm of the baseline hsCRP on the steady state value was less than 1 (equal to 0.67). This indicates that the baseline hsCRP is an imperfect measure for the steady state value, and that the steady state value exposes regression to the mean relative to the baseline value. The effects of the logarithm of the baseline hsCRP on IC50 and Emax were both negative. Thus patients with high hsCRP at baseline are expected to have low IC50 and large maximal reductions. In general, model diagnostics confirmed that the model describes the available hsCRP data well.

The model was then used to simulate expected hsCRP response for a selection of different dosing regimens in a lung cancer patient population. Bootstrapping was applied to construct populations with intended inclusion/exclusion criteria that represent potential lung cancer patient populations. Three different lung cancer patient populations described by baseline hsCRP distribution alone were investigated: all CANTOS patients (scenario 1), confirmed lung cancer patients (scenario 2), and advanced lung cancer patients (scenario 3).

The population parameters and inter-patient variability of the model were assumed to be the same for all three scenarios. The PK/PD relationship on hsCRP observed in the overall CANTOS population was assumed to be representative for lung cancer patients.

The estimator of interest was the probability of hsCRP at end of month 3 being below a cut point, which could be either 2 mg/L or 1.8 mg/L. 1.8 mg/L was the median of hsCRP level at end of month 3 in the CANTOS study. Baseline hsCRP>2 mg/L was one of the inclusion criteria, so it is worthy to explore if hsCRP level at end of month 3 went below 2 mg/L.

A one-compartment model with first order absorption and elimination was established for CANTOS PK data. The model was expressed as ordinary differential equation and RxODE was used to simulate canakinumab concentration time course given individual PK parameters. The subcutaneous canakinumab dose regimens of interest were 300 mg Q12W, 200 mg Q3W, and 300 mg Q4W. Exposure metrics including Cmin, Cmax, AUCs over different selected time periods, and average concentration Cave at steady state were derived from simulated concentration time profiles.

The simulation in Scenario 1 was based on the below information:

Individual canakinumab exposure simulated using RxODE

PD parameters which are components of y_{0,1}, and IC50ᵢ: typical values (THETA(3), THETA(5), THETA(6)), covpars (THETA(4), THETA(7), THETA(8)), and between subject variability (ETA(1), ETA(2), ETA(3))

Baseline hsCRP from all 10,059 CANTOS study patients (baseline hsCRP: mean 6.18 mg/L, standard error of the mean (SEM)=0.10 mg/L)

The prediction interval of the estimator of interest was produced by first randomly sampling 1000 THETA(3)-(8)s from a normal distribution with fixed mean and standard deviation estimated from the population PK/PD model; and then for each set of THETA(3)-(8), bootstrapping 2000 PK exposure, PD parameters ETA(1)-(3), and baseline hsCRP from all CANTOS patients. The 2.5%, 50%, and 97.5% percentile of 1000 estimates were reported as point estimator as well as 95% prediction interval.

The simulation in Scenario 2 was based on the below information:

Individual canakinumab PK exposure simulated using RxODE

PD parameters THETA(3)-(8) and ETA(1)-(3)

Baseline hsCRP from 116 CANTOS patients with confirmed lung cancer (baseline hsCRP: mean=9.75 mg/L, SEM=1.14 mg/L)

The prediction interval of the estimator of interest was produced by first randomly sampling 1000 THETA(3)-(8)s from a normal distribution with fixed mean and standard deviation estimated from the population PKPD model; and then for each set of THETA(3)-(8), bootstrapping 2000 PK exposure, PD parameters ETA(1)-(3) from all CANTOS patients, and bootstrapping 2000 baseline hsCRP from the 116 CANTOS patients with confirmed lung cancer. The 2.5%, 50%, and 97.5% percentile of 1000 estimates were reported as point estimator as well as 95% prediction interval.

In Scenario 3, the point estimator and 95% prediction interval were obtained in a similar manner as for scenario 2. The only difference was bootstrapping 2000 baseline hsCRP values from advanced lung cancer population. There is no individual baseline hsCRP data published in an advanced lung cancer population. An available population level estimate in advanced lung cancer is a mean of baseline hsCRP of 23.94 mg/L with SEM 1.93 mg/L [Vaguliene 2011]. Using this estimate, the advanced lung cancer population was derived from the 116 CANTOS patients with confirmed lung cancer using an additive constant to adjust the mean value to 23.94 mg/L.

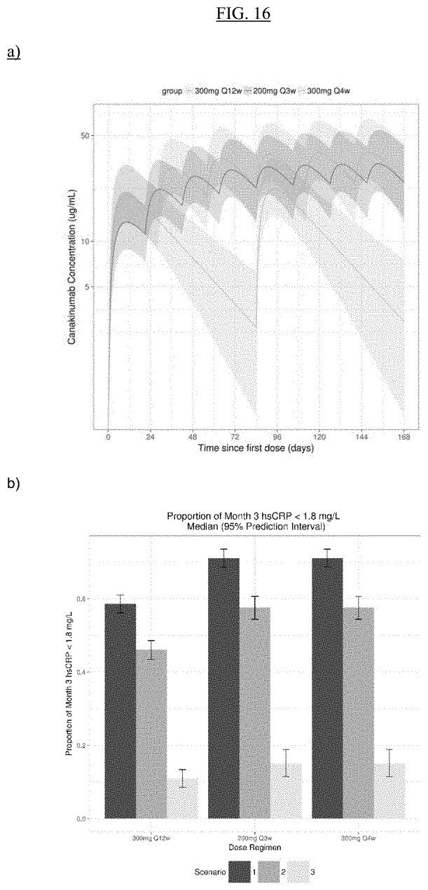

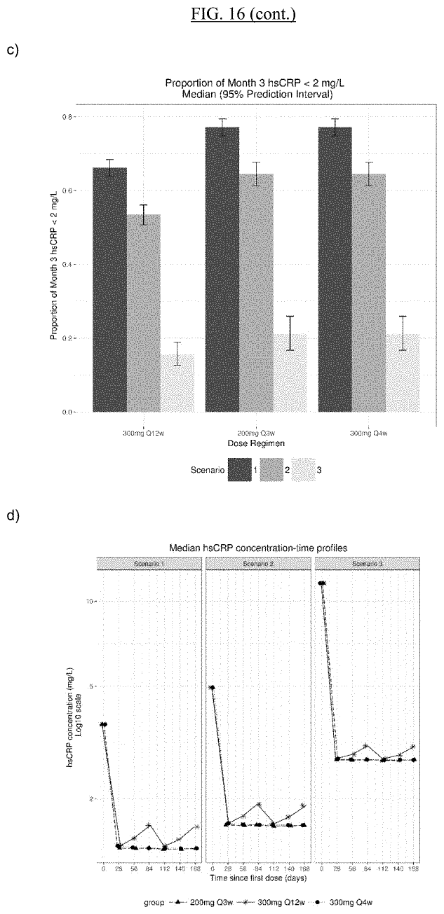

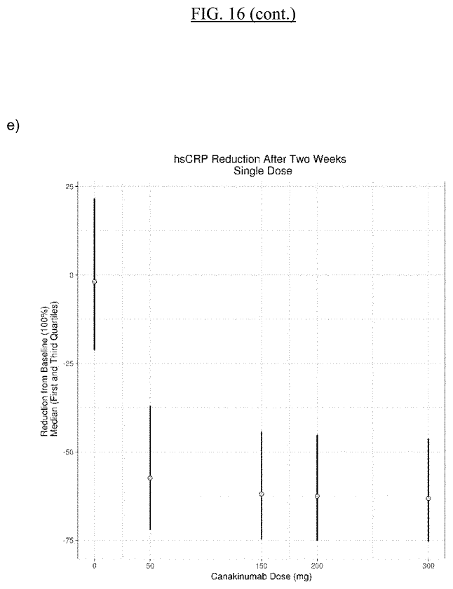

In line with the model, the simulated canakinumab PK was linear. The median and 95% prediction interval of concentration time profiles are plotted in natural logarithm scale over 6 months is shown in FIG. 16, panel a.

The median and 95% prediction intervals of 1000 estimates of proportion of subjects with month 3 hsCRP response under the cut point of 1.8 mg/L and 2 mg/L mhsCRP are reported in FIG. 16, panels b and c. Judging from the simulation data, 200 mg Q3W and 300 mg Q4W perform similarly and better than 300 mg Q12W (top dosing regimen in CANTOS) in terms of decreasing hsCRP at month 3. Going from scenario 1 to scenario 3 towards more severe lung cancer patients, higher baseline hsCRP levels are assumed, and result in smaller probabilities of month 3 hsCRP being below the cut point. FIG. 16, panel d shows how the median hsCRP concentration changes over time for three different doses and FIG. 16, panel e shows the percent reduction from baseline hsCRP after a single dose.

### Example 5A

### PDR001 Plus Canakinumab Treatment Increases Effector Neutrophils in Colorectal Tumors.

RNA sequencing was used to gain insights on the mechanism of action of canakinumab (ACZ885) in cancer. The CPDR001X2102 and CPDR001X2103 clinical trials evaluate the safety, tolerability and pharmacodynamics of spartalizumab (PDR001) in combination with additional therapies. For each patient, a tumor biopsy was obtained prior to treatment, as well as cycle 3 of treatment. In brief, samples were processed by RNA extraction, ribosomal RNA depletion, library construction and sequencing. Sequence reads were aligned by STAR to the hg19 reference genome and Refseq reference transcriptome, gene-level counts were compiled by HTSeq, and sample-level normalization using the trimmed mean of M-values was performed by edgeR.

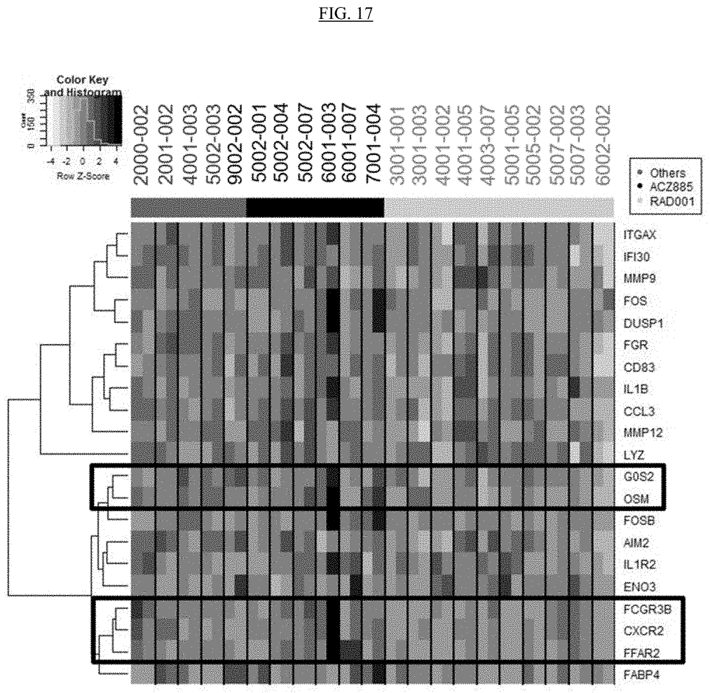

FIG. 17 shows 21 genes that were increased, on average, in colorectal tumors treated with PDR001+canakinumab (ACZ885), but not in colorectal tumors treated with PDR001+everolimus (RAD001). Treatment with PDR001+canakinumab increased the RNA levels of IL1B, as well as its receptor, IL1R2. This observation suggests an on-target compensatory feedback by tumors to increase IL1B RNA levels in response to IL-1β protein blockade.

Notably, several neutrophil-specific genes were increased on PDR001+canakinumab, including FCGR3B, CXCR2, FFAR2, OSM, and G0S2 (indicated by boxes in FIG. 17). The FCGR3B gene is a neutrophil-specific isoform of the CD16 protein. The protein encoded by FCGR3B plays a pivotal role in the secretion of reactive oxygen species in response to immune complexes, consistent with a function of effector neutrophils (Fossati G 2002 Arthritis Rheum 46: 1351). Chemokines that bind to CXCR2 mobilize neutrophils out of the bone marrow and into peripheral sites. In addition, increased CCL3 RNA was observed on treatment with PDR001+canakinumab. CCL3 is a chemoattractant for neutrophils (Reichel C A 2012 Blood 120: 880).

In summary, this contribution of components analysis using RNA-seq data demonstrates that PDR001+canakinumab treatment increases effector neutrophils in colorectal tumors, and that this increase was not observed with PDR001+everolimus treatment.

### Example 5B

Efficacy of Canakinumab (ACZ885) in Combination with Spartalizumab (PDR001) in the Treatment of Cancer.

Patient 5002-004 is a 56 year old man with initially Stage IIC, microsatellite-stable, moderately differentiated adenocarcinoma of the ascending colon (MSS-CRC), diagnosed in June, 2012 and treated with prior regimens.

Prior treatment regimens included:
1. Folinic acid/5-fluoruracil/oxaliplatin in the adjuvant setting
2. Chemoradiation with capecitabine (metastatic setting)
3. 5-fluorouracil/bevacizumab/folinic acid/irinotecan
4. trifluridine and tipiracil
5. Irinotecan
6. Oxaliplatin/5-fluorouracil
57. 5-fluorouracil/bevacizumab/leucovorin
8. 5-fluorouracil

At study entry the patient had extensive metastatic disease including multiple hepatic and bilateral lung metastases, and disease in paraesophageal lymph nodes, retroperitoneum and peritoneum.

The patient was treated with PDR001 400 mg evey four weeks (Q4W) plus 100 mg every eight weeks (Q8W) ACZ885. The patient had stable disease for 6 months of therapy, then with substantial disease reduction and confirmed RECIST partial response to treatment at 10 months. The patient has subsequently developed progressive disease and the dose was increased to 300 mg and then to 600 mg.

### Example 6

### Calculations for Selecting the Dose for Gevokizumab for Cancer Patients.

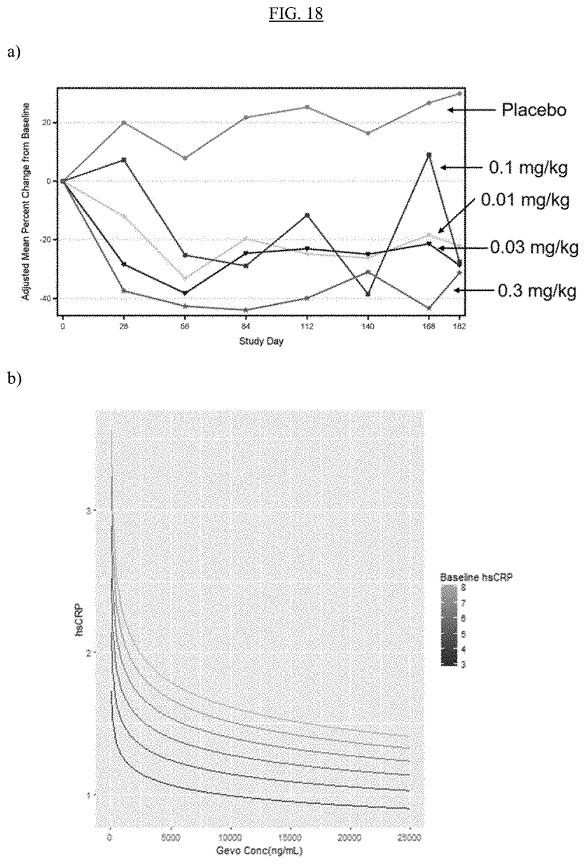

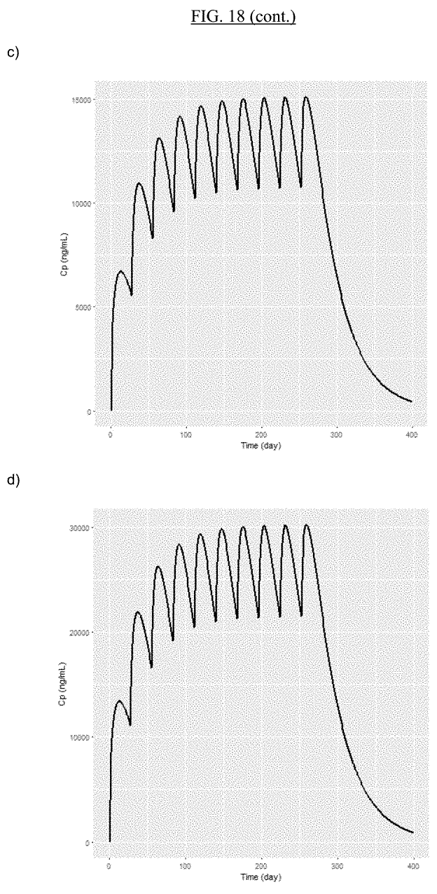

Dose selection for gevokizumab in the treatment of cancer having at least partial inflammatory basis is based on the clinical effective dosings reveals by the CANTOS trial in combination with the available PK data of gevokizumab, taking into the consideration that Gevokizumab (IC50 of ˜2-5 pM) shows a ˜10 times higher in virto potency compared to canakinumab (IC50 of ˜42±3.4 pM). The gevokizumab top dose of 0.3 mg/kg (˜20 mg) Q4W showed reduction of hsCRP in patients that is non-saturating (see FIG. 18, panel a).

Next, a pharmacometric model was used to explore the hsCRP exposure-response relationship, and to extrapolate the clinical data to higher ranges. As clinical data show a linear correlation between the hsCRP concentration and the concentration of gevokizumab (both in log-space), a linear model was used. The results are shown in FIG. 18, panel b. Based on that simulation, a gevokizumab concentration between 10000 ng/mL and 25000 ng/mL is optimal because hsCRP is greatly reduced in this range, and there is only a diminishing return with gevokizumab concentrations above 15000 ng/mL.

Clinical data showed that gevokizumab pharmacokinetics follow a linear two-compartment model with first order absorption after a subcutaneous administration. Bioavailability of gevokizumab is about 56% when administered subcutaneously. Simulation of multiple-dose gevokizumab was carried out for 100 mg every four weeks (see FIG. 18, panel c) and 200 mg every four weeks (see FIG. 18, panel d). The simulations showed that the trough concentration of 100 mg gevokizumab given every four weeks is about 10700 ng/mL. The half-life of gevokizumab is about 35 days. The trough concentration of 200 mg gevokizumab given every four weeks is about 21500 ng/mL.

### Example 7

### Preclinical Data on the Effects of Anti-IL-1Beta Treatment.

Canankinumab, an anti-IL-1beta human IgG1 antibody, cannot directly be evaluated in mouse models of cancer due to the fact that it does not cross-react with mouse IL-1beta. A mouse surrogate anti-IL-1beta antibody has been developed and is being used to evaluate the effects of blocking IL-1beta in mouse models of cancer. This isotype of the surrogate antibody is IgG2a, which is closely related to human IgG1.

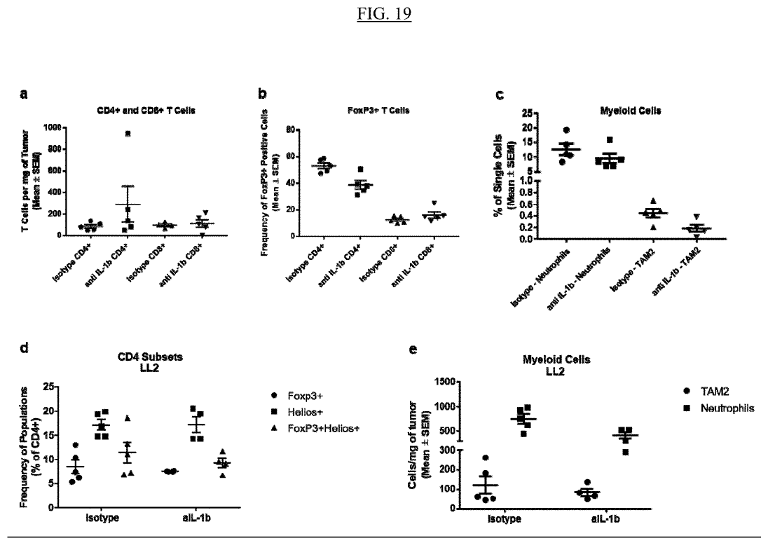

In the MC38 mouse model of colon cancer, modulation of tumor infiltrating lymphocytes (TILs) can be seen after one dose of the anti IL-1beta antibody (FIG. 19, panels a, b, and c). MC38 tumors were subcutaneously implanted in the flank of C57BL/6 mice and when the tumors were between 100-150 mm³, the mice were treated with one dose of either an isotype antibody or the anti IL-1beta antibody. Tumors were then harvested five days after the dose and processed to obtain a single cell suspension of immune cells. The cells were then ex vivo stained and analyzed via flow cytometry. Following a single dose of an IL-1beta blocking antibody, there is an increase in in CD4⁺ T cells infiltrating the tumor and also a slight increase in CD8⁺ T cells (FIG. 19, panel a). The CD8+ T cell increase is slight but may allude to a more active immune response in the tumor microenvironment, which could potentially be enhanced with combination therapies. The CD4⁺ T cells were further subdivided into FoxP3⁺ regulatory T cells (Tregs), and this subset decreases following blockade of IL-1beta (FIG. 19, panel b). Among the myeloid cell populations, blockade of IL-1beta results in a decrease in neutrophils and the M2 subset of macrophages, TAM2 (FIG. 19, panel c). Both neutrophils and M2 macrophages can be suppressive to other immune cells, such as activated T cells (Pillay et al, 2013; Hao et al, 2013; Oishi et al 2016). Taken together, the decrease in Tregs, neutrophils, and M2 macrophages in the MC38 tumor microenvironment following IL-1beta blockade argues that the tumor microenvironment is becoming less immune suppressive.

In the LL2 mouse model of lung cancer, a similar trend towards a less suppressive immune microenvironment can be seen after one dose of an anti-IL-1beta antibody (FIG. 19, panels d and e). LL2 tumors were subcutaneously implanted in the flank of C57BL/6 mice and when the tumors were between 100-150 mm³, the mice were treated with one dose of either an isotype antibody or the anti IL-1beta antibody. Tumors were then harvested five days after the dose and processed to obtain a single cell suspension of immune cells. The cells were then ex vivo stained and analyzed via flow cytometry. There is a decrease in the Treg populations as evaluated by the expression of FoxP3 and Helios (FIG. 19, panel d). FoxP3 and Helios are both used as markers of regulatory T cells, while they may define different subsets of Tregs (Thornton et al, 2016). Similar to the MC38 model, there is a decrease in both neutrophils and M2 macrophages (TAM2) following IL-1beta blockade (FIG. 19, panel e). Again, the decrease in Tregs, neutrophils, and M2 macrophages in the LL2 model following IL-1beta blockade argues that the tumor microenvironment is becoming less immune suppressive.

Mouse models do not always correlate to the same type of cancer in humans due to genetic differences in the origins of the cancer in mice versus humans. However, when examining the infiltrating immune cells, the type of cancer is not always important, as the immune cells are more relevant. In this case, as two different mouse models show a similar decrease in the suppressive microenvironment of the tumor, blocking IL-1beta seems to lead to a less suppressive tumor microenvironment.

TABLE 1
Baseline clinical characteristics of participants in CANTOS among those
who did and did not develop incident cancers during follow-up.

| | No | | Incident | | Incident | |
|---|---|---|---|---|---|---|
| | Incident Cancers | | Non-Lung Cancers | | Lung Cancers | |
| | Placebo | Canakinumab | Placebo | Canakinumab | Placebo | Canakinumab |
| | (N = 3113) | (N = 6286) | (N = 179) | (N = 377) | (N = 61) | (N = 68) |
| Age (yr) | 61.0 | 61.0 | 67.0 | 66.0 | 66.0 | 64.0 |
| Female sex (%) | 26.3 | 25.8 | 22.3 | 22.5 | 14.8 | 26.5 |
| Smoking status (%) | | | | | | |
| Current smoking | 22.3 | 23.6 | 25.7 | 24.7 | 45.9 | 42.6 |
| Past smoking | 48.0 | 46.5 | 55.3 | 48.8 | 50.8 | 54.4 |
| Never smoker | 29.7 | 29.9 | 19.0 | 26.5 | 3.3 | 2.9 |
| Body mass index | 29.8 | 29.8 | 29.0 | 30.1 | 28.3 | 29.7 |
| (kg/m²) | | | | | | |
| Waist circumference | 104 | 104 | 103 | 106 | 106 | 110 |
| (cm) | | | | | | |
| Alcohol use | 4.0 | 3.9 | 5.6 | 4.5 | 3.3 | 2.9 |
| (%, >1/day) | | | | | | |
| Hypertension (%) | 78.8 | 79.6 | 84.9 | 84.9 | 78.7 | 73.5 |
| Diabetes (%) | 39.7 | 39.9 | 40.8 | 44.3 | 45.9 | 38.2 |
| Daily Exercise (%) | 17.5 | 16.9 | 19.6 | 18.1 | 11.5 | 10.3 |
| hsCRP (mg/L) | 4.1 | 4.2 | 4.3 | 4.4 | 6.8 | 6.0 |
| Interleukin-6 (ng/L) | 2.6 | 2.6 | 3.0 | 2.6 | 3.4 | 3.1 |
| Total cholesterol | 161 | 160 | 152 | 153 | 159 | 160 |
| (mg/dL) | | | | | | |
| LDL cholesterol | 83 | 83 | 76 | 75 | 77 | 80 |
| (mg/dL) | | | | | | |
| HDL cholesterol | 44 | 44 | 46 | 45 | 45 | 42 |
| (mg/dL) | | | | | | |
| Triglycerides (mg/dL) | 139 | 140 | 127 | 130 | 140 | 135 |
| eGFR (mL/min/1.73 m²) | 79 | 79 | 75 | 74 | 72 | 78 |

*Shown are median within group levels of characteristics for continuous variables, and percentages for dichotomous variables

TABLE 2
Incidence rates (per 100 person years) and hazard ratios for all
incident cancers, lung cancers, and non-lung cancers in CANTOS.

| | | | | | | P-value |
|---|---|---|---|---|---|---|
| | | | | | | for |
| | | Canakinumab Dose (SC q 3 months) | | | | trend |
| | Placebo | 50 mg | 150 mg | 300 mg | All Doses | across |
| Clinical Outcome | (N = 3344) | (N = 2170) | (N = 2284) | (N = 2263) | (N = 6717) | doses |
| Any Cancer (all) | | | | | | |
| Incident rate, (N) | 1.88 (231) | 1.85 (144) | 1.69 (143) | 1.72 (144) | 1.75 (431) | 0.31 |
| Hazard ratio | 1.00 | 0.99 | 0.90 | 0.91 | 0.93 | |
| 95% CI | (referent) | 0.80-1.22 | 0.73-1.11 | 0.74-1.12 | 0.79-1.09 | |
| P | (referent) | 0.91 | 0.31 | 0.38 | 0.38 | |
| Any Cancer (fatal) | | | | | | |
| Incidence rate, (N) | 0.64 (81) | 0.55 (44) | 0.50 (44) | 0.31 (27) | 0.45 (115) | 0.0007 |
| Hazard ratio | 1.00 | 0.86 | 0.78 | 0.49 | 0.71 | |
| 95% CI | (referent) | 0.59-1.24 | 0.54-1.13 | 0.31-0.75 | 0.53-0.94 | |
| P | (referent) | 0.42 | 0.19 | 0.0009 | 0.016 | |
| Lung Cancer (all) | | | | | | |
| Incidence rate, (N) | 0.49 (61) | 0.35 (28) | 0.30 (26) | 0.16 (14) | 0.27 (68) | <0.0001 |
| Hazard ratio | 1.00 | 0.74 | 0.61 | 0.33 | 0.55 | |
| 95% CI | (referent) | 0.47-1.17 | 0.39-0.97 | 0.18-0.59 | 0.39-0.78 | |
| P | (referent) | 0.20 | 0.034 | <0.0001 | 0.0007 | |
| Lung Cancer (fatal) | | | | | | |
| Incidence rate, (N) | 0.30 (38) | 0.20 (16) | 0.19 (17) | 0.07 (6) | 0.15 (39) | 0.0002 |
| Hazard ratio | 1.00 | 0.67 | 0.64 | 0.23 | 0.51 | |
| 95% CI | (referent) | 0.37-1.20 | 0.36-1.14 | 0.10-0.54 | 0.33-0.80 | |
| P | (referent) | 0.18 | 0.13 | 0.0002 | 0.0026 | |
| Non-Lung Cancer | | | | | | |
| (all) | | | | | | |
| Incidence rate, (N) | 1.46 (179) | 1.55 (121) | 1.44 (122) | 1.60 (134) | 1.53 (377) | 0.54 |
| Hazard ratio | 1.00 | 1.08 | 0.99 | 1.10 | 1.05 | |
| 95% CI | (referent) | 0.85-1.36 | 0.78-1.24 | 0.88-1.37 | 0.88-1.26 | |
| P | (referent) | 0.54 | 0.91 | 0.42 | 0.58 | |
| Non-Lung Cancer | | | | | | |
| (fatal) | | | | | | |
| Incidence rate, (N) | 0.39 (49) | 0.38 (30) | 0.34 (30) | 0.24 (21) | 0.32 (81) | 0.06 |
| Hazard ratio | 1.00 | 0.96 | 0.88 | 0.63 | 0.82 | |
| 95% CI | (referent) | 0.61-1.51 | 0.56-1.39 | 0.38-1.04 | 0.58-1.17 | |
| P | (referent) | 0.86 | 0.60 | 0.07 | 0.28 | |

TABLE 3
Effects of canakinumab as compared to placebo on platelets, leucocytes, neutrophils, and erythrocytes
reported as adverse events and after 12 months of treatment with study drug during CANTOS.

| | Canakinumab Dose (SC q 3 months) | | | | | | | | | | | |
|---|---|---|---|---|---|---|---|---|---|---|---|---|
| | | | | | | | | | | | P-value | P-value |
| | | | | | | | | | | | for | for |
| | | | | | | | | | | | trend | combined |
| | Placebo | | 50 mg | | 150 mg | | 300 mg | | All Doses | | across | dose |
| Adverse Event | (N = 3344) | | (N = 2170) | | (N = 2284) | | (N = 2263) | | (N = 6717) | | doses | groups |
| Thrombocytopenia (AE | 53 | (0.43) | 44 | (0.56) | 46 | (0.54) | 60 | (0.71) | 150 | (0.60) | 0.010 | 0.029 |
| reports)+ | | | | | | | | | | | | |
| Platelets | | | | | | | | | | | | |
| (at 12 months)* | | | | | | | | | | | | |
| Normal N (%) | 2731 | (91.1) | 1741 | (88.9) | 1777 | (87.5) | 1698 | (84.0) | 5216 | (86.8) | <0.0001 | <0.0001 |
| Grade 1 (75,000-<LLN) | 259 | (8.6) | 214 | (10.9) | 252 | (12.4) | 316 | (15.6) | 782 | (13.0) | | |
| Grade 2 (50,000-<75,000) | 6 | (0.20) | 3 | (0.15) | 1 | (0.05) | 6 | (0.30) | 10 | (0.17) | | |
| Grade 3 (25,000-<50,000) | 1 | (0.03) | 0 | (0.00) | 2 | (0.10) | 2 | (0.10) | 4 | (0.07) | | |
| Grade 4 (<25,000) | 0 | (0.00) | 0 | (0.00) | 0 | (0.00) | 0 | (0.00) | 0 | (0.00) | | |
| Leukopenia (AE reports)+ | 30 | (0.24) | 24 | (0.30) | 32 | (0.37) | 44 | (0.52) | 100 | (0.40) | 0.001 | 0.013 |
| Leucocytes (at 12 months)* | | | | | | | | | | | | |
| High (>15,000) | 11 | (0.37) | 9 | (0.46) | 9 | (0.44) | 2 | (0.10) | 20 | (0.33) | 0.09 | 0.56 |
| Normal (3000-<15000) | 2980 | (99.3) | 1944 | (99.2) | 2016 | (99.0) | 2018 | (99.5) | 5978 | (99.2) | | |
| Low (<3000) | 9 | (0.30) | 7 | (0.36) | 11 | (0.54) | 9 | (0.44) | 27 | (0.45) | | |
| Neutropenia (AE reports) | 7 | (0.06) | 4 | (0.05) | 6 | (0.07) | 15 | (0.18) | 25 | (0.10) | 0.003 | 0.17 |
| Neutrophil (at 12 months)* | | | | | | | | | | | | |
| Normal N (%) | 2954 | (99.4) | 1917 | (99.4) | 1991 | (99.1) | 1983 | (99.2) | 5891 | (99.2) | 0.33 | 0.72 |
| Grade 1 (1500-<LLN) | 5 | (0.17) | 4 | (0.21) | 4 | (0.20) | 6 | (0.30) | 14 | (0.24) | | |
| Grade 2 (1000-<1500) | 10 | (0.34) | 6 | (0.31) | 12 | (0.60) | 10 | (0.50) | 28 | (0.47) | | |
| Grade 3 (500-<1000) | 3 | (0.10) | 2 | (0.10) | 2 | (0.10) | 1 | (0.05) | 5 | (0.08) | | |
| Grade 4 (<500) | 0 | (0.00) | 0 | (0.00) | 0 | (0.00) | 0 | (0.00) | 0 | (0.00) | | |
| Anemia (AE reports) | 168 | (1.37) | 63 | (0.80) | 102 | (1.21) | 110 | (1.31) | 275 | (1.11) | 0.57 | 0.033 |
| Erythrocytes (at 12 months)** | | | | | | | | | | | | |
| High (>6.8) | 2 | (0.07) | 1 | (0.05) | 0 | (0.00) | 3 | (0.15) | 4 | (0.07) | 0.31 | 0.62 |
| Normal (3.3-6.8) | 2993 | (99.7) | 1954 | (99.7) | 2031 | (99.8) | 2017 | (99.4) | 6002 | (99.6) | | |
| Low (<3.3) | 6 | (0.20) | 5 | (0.26) | 5 | (0.25) | 9 | (0.44) | 19 | (0.32) | | |

+Standardized MedDRA query
*values per cubic mm
**×10¹²

TABLE 4
Incidence rates (per 100-person years), number (N) of serious adverse events, and
selected on-treatment safety laboratory data (%, N), stratified by study group.

| | Canakinumab Dose (SC q 3 months) | | | | | | | | | | | |
|---|---|---|---|---|---|---|---|---|---|---|---|---|
| | | | | | | | | | | | P-value | P-value |
| | | | | | | | | | | | for | for |
| | | | | | | | | | | | trend | combined |
| Adverse Event or | Placebo | | 50 mg | | 150 mg | | 300 mg | | All Doses | | across | dose |
| Laboratory Parameter | (N = 3344) | | (N = 2170) | | (N = 2284) | | (N = 2263) | | (N = 6717) | | doses | groups |
| Any SAE | 11.8 | (1192) | 11.4 | (740) | 11.6 | (803) | 12.3 | (833) | 11.7 | (2376) | 0.41 | 0.86 |
| Any SAE infection | 2.83 | (339) | 2.97 | (225) | 3.12 | (257) | 3.25 | (265) | 3.11 | (747) | 0.09 | 0.14 |
| Cellulitis | 0.24 | (30) | 0.23 | (18) | 0.37 | (32) | 0.41 | (35) | 0.34 | (85) | 0.018 | 0.10 |
| Pneumonia | 0.89 | (111) | 0.90 | (71) | 0.92 | (79) | 0.97 | (83) | 0.93 | (233) | 0.54 | 0.69 |
| Urinary tract | 0.22 | (27) | 0.18 | (14) | 0.24 | (21) | 0.20 | (17) | 0.21 | (52) | 0.84 | 0.87 |
| Opportunistic | 0.18 | (23) | 0.16 | (13) | 0.15 | (13) | 0.20 | (17) | 0.17 | (43) | 0.97 | 0.78 |
| infections++ | | | | | | | | | | | | |
| Pseudomembranous | 0.03 | (4) | 0.11 | (9) | 0.05 | (4) | 0.12 | (10) | 0.09 | (23) | 0.10 | 0.038 |
| Colitis+ | | | | | | | | | | | | |
| Fatal infection/sepsis | 0.18 | (23) | 0.31 | (25) | 0.28 | (24) | 0.34 | (29) | 0.31 | (78) | 0.09 | 0.023 |
| Other adverse events | | | | | | | | | | | | |
| Injection site reaction++ | 0.23 | (29) | 0.27 | (21) | 0.28 | (24) | 0.30 | (26) | 0.28 | (71) | 0.49 | 0.36 |
| Arthritis+ | 3.20 | (373) | 2.07 | (158) | 2.12 | (176) | 2.43 | (198) | 2.21 | (532) | 0.005 | <0.001 |
| Osteoarthritis | 1.62 | (196) | 1.15 | (90) | 1.10 | (93) | 1.25 | (105) | 1.17 | (288) | 0.038 | 0.001 |
| Gout | 0.80 | (98) | 0.42 | (33) | 0.31 | (27) | 0.37 | (32) | 0.37 | (92) | <0.001 | <0.001 |
| Drug induced liver | 0.18 | (23) | 0.15 | (12) | 0.13 | (11) | 0.05 | (4) | 0.11 | (27) | 0.004 | 0.054 |
| injury (SAE)++ | | | | | | | | | | | | |
| Any Hemorrhage+ | 3.95 | (455) | 3.26 | (244) | 4.09 | (323) | 3.75 | (296) | 3.71 | (863) | 0.94 | 0.31 |
| Hepatic | | | | | | | | | | | | |
| ALT > 3x normal %, (N) | 1.4 | (46) | 1.9 | (42) | 1.9 | (44) | 2.0 | (45) | 2.0 | (131) | 0.19 | 0.059 |
| AST > 3x normal %, (N) | 1.1 | (36) | 1.5 | (32) | 1.5 | (35) | 1.5 | (34) | 1.5 | (101) | 0.29 | 0.11 |
| ALP > 3x normal %, (N) | 0.4 | (15) | 0.5 | (11) | 0.4 | (10) | 0.5 | (12) | 0.5 | (33) | 0.67 | 0.81 |
| Bilirubin > 2x normal %, | 0.8 | (26) | 1.0 | (21) | 0.7 | (15) | 0.7 | (15) | 0.8 | (51) | 0.34 | 0.82 |
| (N) | | | | | | | | | | | | |

+Standardized MedDRA query
++Sponsor categorization of adverse events of special interest

TABLE 5
Proportion of Month 3 hsCRP < cut
point (Median and 95% prediction interval).

| Scenario^{##}/ | | | |
|---|---|---|---|
| Cut point | | | |
| (mg/L) | 300 mg Q12W | 200 mg Q3W | 300 mg Q4W |
| 1/2.0 | 0.6615 | 0.7715 | 0.7715 |
| | (0.6380, 0.6840) | (0.7480, 0.7940) | (0.7480, 0.7940) |
| 1/1.8 | 0.5860 | 0.7110 | 0.7110 |
| | (0.5615, 0.6105) | (0.6860, 0.7355) | (0.6865, 0.7355) |
| 2/2.0 | 0.5355 | 0.6450 | 0.6450 |
| | (0.5075, 0.5610) | (0.6135, 0.6765) | (0.6135, 0.6770) |
| 2/1.8 | 0.4610 | 0.5760 | 0.5760 |
| | (0.4345, 0.4860) | (0.5440, 0.6070) | (0.5440, 0.6065) |
| 3/2.0 | 0.1560 | 0.2110 | 0.2110 |
| | (0.1265, 0.1890) | (0.1674, 0.2595) | (0.1674, 0.2595) |
| 3/1.8 | 0.1095 | 0.1495 | 0.1495 |
| | (0.0850, 0.1340) | (0.1150, 0.1890) | (0.1150, 0.1885) |

^{##}From Scenario 1 to Scenario 3, the severity of lung cancer increased. The means of baseline hsCRP are 6.18 mg/L, 9.75 mg/L, and 23.94 mg/L, respectively.

TABLE S1
Baseline clinical characteristics of CANTOS participants by treatment status.

| | Canakinumab Dose (SC q 3 months) | | | | |
|---|---|---|---|---|---|
| | Placebo | 50 mg | 150 mg | 300 mg | All Doses |
| Characteristic | (N = 3344) | (N = 2170) | (N = 2284) | (N = 2263) | (N = 6717) |
| Mean age (yr) | 61.1 | 61.1 | 61.2 | 61.1 | 61.1 |
| Female sex (%) | 25.9 | 24.9 | 25.2 | 26.8 | 25.6 |
| Current smoking (%) | 22.9 | 24.5 | 23.4 | 23.7 | 23.8 |
| Body mass index (kg/m²) | 29.7 | 29.9 | 29.8 | 29.8 | 29.9 |
| Hypertension (%) | 79.1 | 80.7 | 79.4 | 79.5 | 79.9 |
| Diabetes (%) | 39.9 | 39.4 | 41.8 | 39.2 | 40.1 |
| Qualifying myocardial | | | | | |
| infarction (%) | | | | | |
| STEMI | 54.0 | 56.7 | 53.9 | 53.6 | 54.7 |
| Non-STEMI | 33.9 | 32.7 | 34.2 | 33.6 | 33.5 |
| Unknown/missing | 12.1 | 10.6 | 11.8 | 12.8 | 11.7 |
| History of PCI (%) | 65.6 | 67.0 | 68.1 | 66.7 | 67.3 |
| History of CABG (%) | 14.0 | 13.9 | 14.2 | 14.0 | 14.0 |
| History of congestive heart | 21.6 | 20.8 | 20.9 | 23.1 | 21.6 |
| failure (%) | | | | | |
| Lipid lowering therapy (%) | 93.7 | 94.0 | 92.7 | 93.5 | 93.3 |
| Renin-angiotensin inhibitors | 79.8 | 79.3 | 79.8 | 79.6 | 79.3 |
| (%) | | | | | |
| Anti-ischemia agents* (%) | 92.1 | 91.0 | 91.2 | 91.1 | 91.0 |
| hsCRP (mg/L) | 4.1 | 4.25 | 4.25 | 4.15 | 4.2 |
| Il-6 (ng/L) | 2.61 | 2.53 | 2.56 | 2.59 | 2.56 |
| Total cholesterol (mg/dL) | 160.5 | 159.0 | 159.0 | 161.0 | 159.7 |
| LDL cholesterol (mg/dL) | 82.8 | 81.2 | 82.4 | 83.5 | 82.0 |
| HDL cholesterol (mg/dL) | 44.5 | 43.7 | 43.7 | 44.0 | 43.7 |
| Triglycerides (mg/dL) | 139.0 | 139.9 | 139.1 | 138.2 | 139.1 |
| eGFR (mL/min/1.73 m²) | 79.0 | 79.0 | 79.0 | 78.0 | 78.5 |
| Loss to follow-up N, (%) | 9 (0.27) | 9 (0.41) | 5 (0.22) | 4 (0.18) | 18 (0.27) |

STEMI = ST elevation myocardial infarction;
PCI = percutaneous coronary intervention;
CABG = coronary bypass graft surgery;
hsCRP = high sensitivity C-reactive protein;
HDL = high density lipoprotein cholesterol;
LDL = low density lipoprotein cholesterol;
eGFR = estimated glomerular filtration rate
*Beta-blocking agents, nitrates, or calcium channel blocking agents
Median values are presented for all measured plasma variables and body mass index

TABLE S2
Incidence rates (per 100 person years) and hazard ratios
for lung cancers among current and past smokers.

| | | | | | | P-value |
|---|---|---|---|---|---|---|
| | | | | | | for |
| | | Canakinumab Dose (SC q 3 months) | | | | trend |
| | Placebo | 50 mg | 150 mg | 300 mg | All Doses | across |
| | (N = 3344) | (N = 2170) | (N = 2284) | (N = 2263) | (N = 6717) | doses |
| Lung Cancer, | | | | | | |
| Current Smokers | | | | | | |
| Incident rate, (N) | 0.97 (28) | 0.46 (9) | 0.75 (15) | 0.25 (5) | 0.49 (29) | 0.005 |
| Hazard ratio | 1.00 | 0.49 | 0.76 | 0.25 | 0.50 | |
| 95% CI | (referent) | 0.23-1.05 | 0.40-1.42 | 0.10-0.65 | 0.30-0.84 | |
| P | (referent) | 0.06 | 0.38 | 0.002 | 0.007 | |
| Lung Cancer, | | | | | | |
| Past Smokers | | | | | | |
| Incidence rate, (N) | 0.51 (31) | 0.48 (18) | 0.25 (10) | 0.23 (9) | 0.31 (37) | 0.006 |
| Hazard ratio | 1.00 | 0.95 | 0.48 | 0.44 | 0.61 | |
| 95% CI | (referent) | 0.53-1.71 | 0.24-0.99 | 0.21-0.92 | 0.38-0.99 | |
| P | (referent) | 0.87 | 0.041 | 0.025 | 0.043 | |

TABLE S3
Incidence rates per 100 person years and (number) for lung cancer
types and other site-specific non-lung cancers in CANTOS.

| | Canakinumab Dose (SC q 3 months) | | | | | | |
|---|---|---|---|---|---|---|---|
| | | | | | | P-value | P-value |
| | | | | | | for | for |
| | | | | | All | trend | combined |
| | Placebo | 50 mg | 150 mg | 300 mg | Doses | across | dose |
| Cancer Site or Type | (N = 3344) | (N = 2170) | (N = 2284) | (N = 2263) | (N = 6717) | doses | groups |

Lung Cancers

| | | | | | | | | | | | | |
|---|---|---|---|---|---|---|---|---|---|---|---|---|
| Adenocarcinoma or | 0.41 | (52) | 0.33 | (26) | 0.27 | (23) | 0.12 | (10) | 0.23 | (59) | <0.001 | 0.002 |
| poorly differentiated large | | | | | | | | | | | | |
| cell carcinoma or | | | | | | | | | | | | |
| unspecified | | | | | | | | | | | | |
| Squamous cell lung | 0.03 | (4) | 0.01 | (1) | 0.02 | (2) | 0.03 | (3) | 0.02 | (6) | 0.74 | 0.65 |
| carcinoma | | | | | | | | | | | | |
| Small cell lung cancer | 0.04 | (5) | 0.01 | (1) | 0.01 | (1) | 0.01 | (1) | 0.01 | (3) | NA | NA |

| | | | | | | | | |
|---|---|---|---|---|---|---|---|---|
| Pleural cancer | 0.01 | (1) | 0 | 0 | 0 | 0 | NA | NA |

Other Cancer Sites

| Skin | | | | | | | | | | | | |
|---|---|---|---|---|---|---|---|---|---|---|---|---|
| Basal cell carcinoma | 0.18 | (23) | 0.28 | (22) | 0.29 | (25) | 0.21 | (18) | 0.26 | (65) | 0.80 | 0.16 |
| Squamous cell skin | 0.16 | (20) | 0.10 | (8) | 0.15 | (13) | 0.27 | (23) | 0.17 | (44) | 0.036 | 0.74 |
| cancer | | | | | | | | | | | | |
| Melanoma | 0.02 | (3) | 0.08 | (6) | 0.06 | (5) | 0.06 | (5) | 0.06 | (16) | 0.44 | 0.11 |
| Other | 0.06 | (8) | 0 | | 0.06 | (5) | 0.02 | (2) | 0.03 | (7) | 0.41 | 0.10 |
| Gastrointenstinal | | | | | | | | | | | | |
| Oral cavity/tongue | 0.02 | (3) | 0.03 | (2) | 0.05 | (4) | 0.02 | (2) | 0.03 | (8) | 0.99 | 0.69 |
| Esophageal | 0.06 | (8) | 0.08 | (6) | 0.03 | (3) | 0.08 | (7) | 0.06 | (16) | 0.80 | 1.00 |
| Gastric | 0.08 | (10) | 0.04 | (3) | 0.02 | (2) | 0.06 | (5) | 0.04 | (10) | 0.54 | 0.11 |
| Colorectal | 0.16 | (20) | 0.25 | (20) | 0.19 | (16) | 0.21 | (18) | 0.21 | (54) | 0.66 | 0.26 |
| Biliary | 0.01 | (1) | 0.03 | (2) | 0.03 | (3) | 0 | | 0.02 | (5) | NA | NA |
| Appendiceal | 0.01 | (1) | 0 | | 0 | | 0.01 | (1) | 0.00 | (1) | NA | NA |
| Pancreatic | 0.06 | (8) | 0.04 | (3) | 0.06 | (5) | 0.06 | (5) | 0.05 | (13) | 0.95 | 0.64 |
| Hematopoetic | | | | | | | | | | | | |
| Lymphoma | 0.06 | (7) | 0.04 | (3) | 0.05 | (4) | 0.07 | (6) | 0.05 | (13) | 0.57 | 0.87 |
| Leukemia | 0.01 | (1) | 0.01 | (1) | 0.02 | (2) | 0.01 | (1) | 0.02 | (4) | NA | NA |
| Multiple myeloma | 0.02 | (2) | 0 | | 0 | | 0.02 | (2) | 0.01 | (2) | NA | NA |
| Endocrine | | | | | | | | | | | | |
| Thyroid | 0.03 | (4) | 0.01 | (1) | 0.02 | (2) | 0.01 | (1) | 0.02 | (4) | NA | NA |
| Adrenal | 0.02 | (2) | 0 | | 0.01 | (1) | 0.01 | (1) | 0.01 | (2) | NA | NA |
| Genitourinary | | | | | | | | | | | | |
| Bladder | 0.06 | (8) | 0.10 | (8) | 0.08 | (7) | 0.13 | (11) | 0.10 | (26) | 0.21 | 0.23 |
| Prostate | 0.15 | (19) | 0.19 | (15) | 0.16 | (14) | 0.17 | (15) | 0.17 | (44) | 0.85 | 0.60 |
| Testicular | 0 | | 0 | | 0.01 | (1) | 0 | | 0.00 | (1) | NA | NA |
| Ovarian | 0 | | 0 | | 0.01 | (1) | 0 | | 0.00 | (1) | NA | NA |
| Endometrial/Uterine | 0.01 | (1) | 0.03 | (2) | 0 | | 0.03 | (3) | 0.02 | (5) | NA | NA |
| Cervical | 0.01 | (1) | 0 | | 0.01 | (1) | 0 | | 0.00 | (1) | NA | NA |
| Breast | 0.06 | (8) | 0.09 | (7) | 0.05 | (4) | 0.06 | (5) | 0.06 | (16) | 0.63 | 0.99 |
| Kidney | 0.06 | (8) | 0.13 | (10) | 0.07 | (6) | 0.07 | (6) | 0.09 | (22) | 0.77 | 0.44 |
| Liver | 0.07 | (9) | 0.04 | (3) | 0.06 | (5) | 0.03 | (3) | 0.04 | (11) | 0.38 | 0.26 |
| Central Nervous System | 0.01 | (1) | 0.04 | (3) | 0.05 | (4) | 0.03 | (3) | 0.04 | (10) | 0.33 | 0.09 |
| Sarcoma/Bone | 0.03 | (4) | 0.01 | (1) | 0 | | 0.01 | (1) | 0.01 | (2) | NA | NA |
| Other | 0.09 | (11) | 0.08 | (6) | 0.06 | (5) | 0.02 | (2) | 0.05 | (13) | 0.047 | 0.19 |

NA—tests for significance not performed if event number <10.

TABLE S4
Sensitivity analysis of incidence rates (per 100 person years) and hazard ratios
based upon all reported cancers in CANTOS rather than on adjudicated cancers.

| | | | | | | P-value |
|---|---|---|---|---|---|---|
| | | | | | | for |
| | | Canakinumab Dose (SC q 3 months) | | | | trend |
| | Placebo | 50 mg | 150 mg | 300 mg | All Doses | across |
| Clinical Outcome | (N = 3344) | (N = 2170) | (N = 2284) | (N = 2263) | (N = 6717) | doses |
| Any Reported | | | | | | |
| Cancer (all) | | | | | | |
| Incident rate, (N) | 1.93 (237) | 1.88 (146) | 1.76 (148) | 1.78 (149) | 1.80 (443) | 0.38 |
| Hazard ratio | 1.00 | 0.98 | 0.91 | 0.92 | 0.93 | |
| 95% CI | (referent) | 0.79-1.20 | 0.74-1.11 | 0.75-1.13 | 0.80-1.09 | |
| P | (referent) | 0.82 | 0.35 | 0.43 | 0.39 | |
| Any Reported | | | | | | |
| Cancer (fatal) | | | | | | |
| Incidence rate, (N) | 0.64 (81) | 0.55 (44) | 0.50 (44) | 0.31 (27) | 0.45 (115) | 0.0007 |
| Hazard ratio | 1.00 | 0.86 | 0.78 | 0.49 | 0.71 | |
| 95% CI | (referent) | 0.59-1.24 | 0.54-1.13 | 0.31-0.75 | 0.53-0.94 | |
| P | (referent) | 0.42 | 0.19 | 0.0009 | 0.016 | |
| Reported Lung | | | | | | |
| Cancer (all) | | | | | | |
| Incidence rate, (N) | 0.50 (62) | 0.35 (28) | 0.31 (27) | 0.20 (17) | 0.29 (72) | 0.0003 |
| Hazard ratio | 1.00 | 0.73 | 0.62 | 0.39 | 0.58 | |
| 95% CI | (referent) | 0.47-1.15 | 0.40-0.98 | 0.23-0.67 | 0.41-0.81 | |
| P | (referent) | 0.17 | 0.040 | 0.0004 | 0.0013 | |
| Reported Lung | | | | | | |
| Cancer (fatal) | | | | | | |
| Incidence rate, (N) | 0.30 (38) | 0.20 (16) | 0.19 (17) | 0.07 (6) | 0.15 (39) | 0.0002 |
| Hazard ratio | 1.00 | 0.67 | 0.64 | 0.23 | 0.51 | |
| 95% CI | (referent) | 0.37-1.20 | 0.36-1.14 | 0.10-0.54 | 0.33-0.80 | |
| P | (referent) | 0.18 | 0.13 | 0.0002 | 0.0026 | |
| Reported Non-Lung | | | | | | |
| Cancer (all) | | | | | | |
| Incidence rate, (N) | 1.54 (189) | 1.62 (126) | 1.53 (129) | 1.66 (139) | 1.60 (394) | 0.60 |
| Hazard ratio | 1.00 | 1.06 | 0.99 | 1.08 | 1.04 | |
| 95% CI | (referent) | 0.85-1.33 | 0.79-1.24 | 0.87-1.34 | 0.88-1.24 | |
| P | (referent) | 0.62 | 0.93 | 0.50 | 0.65 | |
| Reported Non-Lung | | | | | | |
| Cancer (fatal) | | | | | | |
| Incidence rate, (N) | 0.41 (52) | 0.40 (32) | 0.37 (32) | 0.27 (23) | 0.34 (87) | 0.07 |
| Hazard ratio | 1.00 | 0.97 | 0.89 | 0.64 | 0.83 | |
| 95% CI | (referent) | 0.63-1.51 | 0.57-1.38 | 0.39-1.05 | 0.59-1.17 | |
| P | (referent) | 0.91 | 0.59 | 0.08 | 0.29 | |

### Specific Embodiments

While various specific embodiments have been illustrated and described, it will be appreciated that various changes can be made without departing from the spirit and scope of the disclosure(s). The present disclosure is exemplified by the numbered embodiments set forth below.
1. An IL-1β binding antibody or a functional fragment thereof for use in a patient in need thereof in the treatment and/or prevention of a cancer having at least partial inflammatory basis.
2. An IL-1β binding antibody or a functional fragment thereof for use in a patient in need thereof in the treatment of a cancer having at least partial inflammatory basis.
103. The embodiment of claim 1 or 2, wherein said cancer having at least partial inflammatory basis is selected from the list consisting of lung cancer, especially NSCLC, colorectal cancer (CRC), melanoma, gastric cancer (including esophageal cancer), renal cell carcinoma (RCC), breast cancer, prostate cancer, head and neck cancer, bladder cancer, hepatocellular carcinoma (HCC), ovarian cancer, cervical cancer, endometrial cancer, pancreatic cancer, neuroendocrine cancer, multiple myeloma, acute myeloblastic leukemia (AML), and biliary tract cancer.
4. The first or second embodiment above, wherein said cancer having at least partial inflammatory basis is selected from the list consisting of lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer.
5. The first or second embodiment above, wherein said cancer having at least partial inflammatory basis is colorectal cancer (CRC).
6. The first or second embodiment above, wherein said cancer having at least partial inflammatory basis is renal cell carcinoma (RCC).
257. The first or second embodiment above, wherein said cancer having at least partial inflammatory basis is breast cancer.
   8. The first or second embodiment above, wherein said cancer having at least partial inflammatory basis is lung cancer, preferably non-small cell lung cancer (NSCLC).
   9. Any of the preceding embodiments, wherein said patient has high sensitivity C-reactive protein (hsCRP) equal to or greater than about 2 mg/L before first administration of said IL-1β binding antibody or functional fragment thereof.
   10. Any of the preceding embodiments, wherein said patient has high sensitivity C-reactive protein (hsCRP) equal to or greater than 4 mg/L before first administration of said IL-1β binding antibody or functional fragment thereof
   11. Any of the preceding embodiments, wherein said patient has high sensitivity C-reactive protein (hsCRP) equal to or greater than 10 mg/L before first administration of said IL-1β binding antibody or functional fragment thereof
   12. Any of the preceding embodiments, wherein the high sensitivity C-reactive protein (hsCRP) level of said patient has reduced to below about 3.5 mg/L assessed at least about 3 months after first administration of the IL-1β binding antibody or functional fragment thereof.
   13. Any of the preceding embodiments, wherein the high sensitivity C-reactive protein (hsCRP) level of said patient has reduced to below about 2.3 mg/L, preferably to below about 2 mg/L, preferably to below about 1.8 mg/L, assessed at least about 3 months after first administration of the IL-1β binding antibody or functional fragment thereof.
   14. Any of the preceding embodiments, wherein the high sensitivity C-reactive protein (hsCRP) level of said patient has reduced by at least 20% compared to baseline assessed at least about 3 months after first administration of the IL-1β binding antibody or functional fragment thereof
   15. Any of the preceding embodiments, wherein the interleukin-6 (IL-6) level of said patient has reduced by at least 20% compared to baseline assessed at least about 3 months after first administration of the IL-1β binding antibody or functional fragment thereof.
   16. Any of the preceding embodiments, wherein said use comprises administering a dose of about 90 mg to about 450 mg of the IL-1β binding antibody or a functional fragment thereof per treatment.
   17. Any of the preceding embodiments, wherein said use comprises administering said IL-1β binding antibody or a functional fragment thereof every two, three or four weeks (monthly).
   18. Any of the preceding embodiments, the second administration of said IL-1β binding antibody or a functional fragment thereof is at most two weeks, preferably two weeks apart from the first administration.
   19. Any of the preceding embodiments, wherein said IL-1β binding antibody is canakinumab.
   20. Any of the preceding embodiments comprising administering about 200 mg to about 450 mg canakinumab per treatment to said patient.
   21. Any of the preceding embodiments comprising administering at least 150 mg canakinumab per treatment to said patient.
   22. The embodiments set forth in 19 or 21 comprising administering about 200 mg of canakinumab to said patient.
   23. The embodiments set forth in any one of 16-22, wherein canakinumab is administered every three weeks.
   24. The embodiments set forth in any one of 16-22, wherein canakinumab is administered every four weeks (monthly).
   25. The embodiments set forth in any one of 16-24, wherein canakinumab is administered subcutaneously.
   26. The embodiments set forth in any one of 16-25, wherein canakinumab is administered in a liquid form contained in a prefilled syringe or as a lyophilized form for reconstitution.
   27. Canakinumab for use in a patient in need thereof in the treatment of a cancer having at least partial inflammatory basis, preferably lung cancer, wherein said use comprises administering a dose of 200 mg of canakinumab subcutaneously every three week.
   28. The embodiments set forth in any one of 1-18, wherein said IL-1β binding antibody is gevokizumab (XOMA-052).
   29. The 28^{th} embodiment, wherein said use comprises administering 90 mg to 270 mg gevokizumab per treatment to said patient.
   30. The 28^{th} embodiment, comprising administering about 90 mg to about 120 mg of gevokizumab to said patient.
   31. The embodiments set forth in any one of 28-30, wherein gevokizumab is administered every three weeks.
   32. The embodiments set forth in any one of 28-30, wherein gevokizumab is administered every four weeks (monthly).
   33. The embodiments set forth in any one of 28-32, wherein gevokizumab is administered subcutaneously.
   34. The embodiments set forth in any one of 28-32, wherein gevokizumab is administered intravenously.
   35. Gevokizumab for use in a patient in need thereof in the treatment of a cancer having at least partial inflammatory basis, wherein said use comprises administering a dose of 120 mg of gevokizumab intravenously every four weeks (monthly).
   36. The embodiment set forth in 35, wherein said cancer having at least partial inflammatory basis is selected from the list consisting of lung cancer, especially NSCLC, colorectal cancer, melanoma, gastric cancer (including esophageal cancer), renal cell carcinoma (RCC), breast cancer, hepatocellular carcinoma (HCC), prostate cancer, bladder cancer, AML, multiple myeloma and pancreatic cancer.
   37. Any of the previous embodiments, wherein said IL-1β binding antibody or a functional fragment thereof is administered in combination with one or more chemotherapeutic agent; wherein preferably said IL-1β binding antibody or a functional fragment thereof is canakinumab or gevokizumab.
   38. The embodiment set forth in 37, wherein said one or more chemotherapeutic agent is the standard of care agent for said cancer.
   39. The embodiments set forth in 37 or 38, wherein said one or more chemotherapeutic agent is the standard of care agent for lung cancer, especially for NSCLC.
   40. The embodiments set forth in any one of 37 to 39, wherein said one or more chemotherapeutic agent is a platinum based chemotherapy or a platinum-based doublet chemotherapy (PT-DC).
   41. The embodiments set forth in any one of 37 to 40, wherein said one or more chemotherapeutic agent is a tyrosine kinase inhibitor.
   42. The embodiments set forth in any one of 37 to 41, wherein said one or more chemotherapeutic agent is a checkpoint inhibitor.
   43. The embodiments set forth in any one of 37 to 42, wherein said one or more chemotherapeutic agent is a PD-1 or PD-L1 inhibitor preferably selected from the group consisting of nivolumab, pembrolizumab, atezolizumab, durvalumab, avelumab and spartalizumab (PDR-001).
   44. Any of the previous embodiments, wherein said IL-1β binding antibody or a functional fragment thereof is used, alone or preferably in combination, in the prevention of recurrence or relapse of cancer having at least a partial inflammatory basis in a subject after said cancer has been surgically removed.
   45. The embodiment set forth in 44, wherein said cancer with partial inflammatory basis is lung cancer.
   46. Any of the preceding embodiments, wherein said IL-1β binding antibody or a functional fragment thereof is used, alone or preferably in combination, as the first line treatment of lung cancer, especially NSCLC.
   47. Any of the preceding embodiments, wherein said IL-1β binding antibody or a functional fragment thereof is used, alone or preferrably in combination, as the second or third line treatment of lung cancer, especially NSCLC.
   48. The embodiments set forth in any one of 37 to 47, wherein said IL-1β binding antibody or a functional fragment thereof is canakinumab, wherein said patient is a smoker.
   49. An IL-1β binding antibody or a functional fragment thereof for use in the prevention of lung cancer in a patient, wherein said patient has a high sensitive C-reactive protein (hsCRP) level of equal or greater than 2 mg/L.
   50. The embodiment set forth in 49, wherein said hsCRP level is equal to or greater than 4 mg/L.
   51. The embodiments set forth in any one of 49-50, wherein said IL-1β binding antibody or a functional fragment thereof is canakinumab or a functional fragment thereof or gevokizumab or a functional fragment thereof.
   52. Any of the preceding embodiments, wherein gevokizumab or a functional fragment thereof is administered in combination with one or more chemotherapeutic agent.
   53. The embodiment set forth in 52, wherein said one or more chemotherapeutic agent is the standard of care agent for colorectal cancer (CRC).
   54. The embodiments set forth in 52 or 53, wherein said one or more chemotherapeutic agent is a general cytotoxic agent, wherein preferably said general cytotoxic agent is selected from the list consisting of FOLFOX, FOLFIRI, capecitabine, 5-fluorouracil, irinotecan and oxaliplatin.
   55. The embodiments set forth in 52 or 53, wherein said one or more chemotherapeutic agent is a VEGF inhibitor, wherein preferably said VEGF inhibitor is selected from the list consisting of bevacizumab, ramucirumab and ziv-aflibercept.
   56. The embodiments set forth in any one of 52 to 55, wherein gevokizumab or a functional fragment thereof is administered in combination with FOLFIRI plus bevacizumab or FOLFOX plus bevacizumab.
   57. The embodiments set forth in any one of 52 to 56, wherein said one or more chemotherapeutic agent is a checkpoint inhibitor.
   58. The embodiments set forth in any one of 52 to 57, wherein said one or more chemotherapeutic agent is a PD-1 or PD-L1 inhibitor preferably selected from the group consisting of nivolumab, pembrolizumab, atezolizumab, avelumab, durvalumab, and spartalizumab (PDR-001).
   59. The embodiments set forth in any one of 52 to 58, wherein gevokizumab or a functional fragment thereof is used, alone or preferably in combination, in the prevention of recurrence or relapse of colorectal cancer in a patient after said cancer has been surgically removed.
   60. The embodiments set forth in any one of 52 to 59, wherein gevokizumab or a functional fragment thereof is used, alone or preferably in combination, as the first line treatment of colorectal cancer.
   61. The embodiments set forth in any one of 52 to 59, wherein gevokizumab or a functional fragment thereof is used, alone or preferably in combination, as the second or third line treatment of colorectal cancer.
   62. The embodiment set forth in 52, wherein said one or more chemotherapeutic agent is the standard of care agent for renal cell carcinoma (RCC).
   63. The embodiments set forth in 52 or 62, wherein said one or more chemotherapeutic agent is a CTLA-4 checkpoint inhibitor, wherein preferably said CTLA-4 checkpoint inhibitor is ipilimumab.
   64. The embodiments set forth in any one of 52 and 62-63, wherein said one or more chemotherapeutic agent is everolimus.
   65. The embodiments set forth in any one of 52 and 62-64, wherein said one or more chemotherapeutic agent is a checkpoint inhibitor.
   66. The embodiments set forth in any one of 52 and 62-65, wherein said one or more chemotherapeutic agent is a PD-1 or PD-L1 inhibitor preferably selected from the group consisting of nivolumab, pembrolizumab, atezolizumab, avelumab, durvalumab and spartalizumab (PDR-001).
   67. The embodiments set forth in any one of 52 and 62-66, wherein said checkpoint inhibitor is nivolumab.
   68. The embodiments set forth in any one of 52 and 62-67, wherein said one or more chemotherapeutic agent are nivolumab plus ipilimumab.
   69. The embodiments set forth in any one of 52 and 62-68, wherein said one or more chemotherapeutic agent is cabozantinib.
   70. The embodiments set forth in any one of 52 and 62-69, wherein gevokizumab or a functional fragment thereof is used, alone or preferably in combination, in the prevention of recurrence or relapse of renal cell carcinoma (RCC) in a patient after said cancer has been surgically removed.
   71. The embodiments set forth in any one of 52 and 62-70, wherein gevokizumab or a functional fragment thereof is used, alone or preferably in combination, in first line treatment of renal cell carcinoma (RCC).
   72. The embodiments set forth in any one of 52 and 62-70, wherein gevokizumab or a functional fragment thereof is used, alone or preferably in combination, in second or third line of renal cell carcinoma (RCC).
   73. The embodiment set forth in 52, wherein said one or more chemotherapeutic agent is the standard of care agent for gastric cancer (including esophageal cancer).
   74. The embodiments set forth in any one of 52 and 73, wherein said one or more chemotherapeutic agent is a mitotic inhibitor, preferably taxane, wherein preferably said taxane is selected from paclitaxel and docetaxel.
   75. The embodiments set forth in any one of 52 and 73-74, wherein said one or more chemotherapeutic agent wherein said one or more chemotherapeutic agent are paclitaxel and ramucirumab.
   76. The embodiments set forth in any one of 52 and 73-75, wherein said one or more chemotherapeutic agent is a checkpoint inhibitor.
   77. The embodiments set forth in any one of 52 and 73-76, wherein said one or more chemotherapeutic agent is a PD-1 or PD-L1 inhibitor preferably selected from the group consisting of nivolumab, pembrolizumab, atezolizumab, durvalumab, avelumab and spartalizumab (PDR-001).
   78. The embodiments set forth in 76 or 77, wherein said checkpoint inhibitor is nivolumab.
   79. The embodiments set forth in any one of 52 and 73-78, wherein said one or more chemotherapeutic agent are nivolumab and ipilimumab.
   80. The embodiments set forth in any one of 52 and 73-79, wherein gevokizumab or a functional fragment thereof is used, alone or preferably in combination, in the prevention of recurrence or relapse of in a patient after said gastric cancer (including esophageal cancer) has been surgically removed.
   81. The embodiments set forth in any one of 52 and 73-80, wherein gevokizumab or a functional fragment thereof is used, alone or preferably in combination, as the first line treatment of gastric cancer (including esophageal cancer).
   82. The embodiments set forth in any one of 52 and 73-81, wherein gevokizumab or a functional fragment thereof is used, alone or preferably in combination, as the second or third line treatment of gastric cancer (including esophageal cancer).

### REFERENCES

1. Coussens L M, Werb Z. Inflammation and cancer. Nature 2002; 420:860-7.
2. Apte R N, Dotan S, Elkabets M, White M R, Reich E, Carmi Y, Song X, Dvozkin T, Krelin Y, Voronov E. The involvement of IL-1 in tumorigenesis, tumor invasiveness, metastasis and tumor-host interactions. Cancer Metastasis Rev 2006; 25:387-408.
3. Porta C, Larghi P, Rimoldi M, Totaro M G, Allavena P, Mantovani A, Sica A. Cellular and molecular pathways linking inflammation and cancer. Immunobiology 2009; 214:761-77.
4. Balkwill F R, Mantovani A. Cancer-related inflammation: common themes and therapeutic opportunities. Semin Cancer Biol 2012; 22:33-40.
5. O'Callaghan D S, O'Donnell D, O'Connell F, O'Byrne K J. The role of inflammation in the pathogenesis of non-small cell lung cancer. J Thorac Oncol 2010; 5:2024-36.
6. Lee J M, Yanagawa J, Peebles K A, Sharma S, Mao J T, Dubinett S M. Inflammation in lung carcinogenesis: new targets for lung cancer chemoprevention and treatment. Crit Rev Oncol Hematol 2008; 66:208-17.
7. Dostert C, Petrilli V, Van Bruggen R, Steele C, Mossman B T, Tschopp J. Innate immune activation through Nalp3 inflammasome sensing of asbestos and silica. Science 2008; 320:674-7.
8. Gasse P, Mary C, Guenon I, Noulin N, Charron S, Schnyder-Candrian S, Schnyder B, Akira S, Quesniaux V F, Lagente V, Ryffel B, Couillin I. IL-1R1/MyD88 signaling and the inflammasome are essential in pulmonary inflammation and fibrosis in mice. J Clin Invest 2007; 117:3786-99.
9. Voronov E, Shouval D S, Krelin Y, Cagnano E, Benharroch D, Iwakura Y, Dinarello C A, Apte R N. IL-1 is required for tumor invasiveness and angiogenesis. Proc Natl Acad Sci USA 2003; 100:2645-50.
10. Dinarello C A, Simon A, van der Meer J W. Treating inflammation by blocking interleukin-1 in a broad spectrum of diseases. Nat Rev Drug Discov 2012; 11:633-52.
11. Dinarello C A. Why not treat human cancer with interleukin-1 blockade? Cancer Metastasis Rev 2010; 29:317-29.
12. Apte R N, Voronov E. Is interleukin-1 a good or bad ‘guy’ in tumor immunobiology and immunotherapy? Immunol Rev 2008; 222:222-41.
13. Lewis A M, Varghese S, Xu H, Alexander H R. Interleukin-1 and cancer progression: the emerging role of interleukin-1 receptor antagonist as a novel therapeutic agent in cancer treatment. J Transl Med 2006; 4:48.
14. Ridker P M, Thuren T, Zalewski A, Libby P. Interleukin-1β inhibition and the prevention of recurrent cardiovascular events: rationale and design of the Canakinumab Anti-inflammatory Thrombosis Outcomes Study (CANTOS). Am Heart J 2011; 162:597-605.
15. Ridker P M, Howard C P, Walter V, Everett B, Libby P, Hensen J, Thuren T. Effects of interleukin-1B inhibition with canakinumab on hemoglobin A1c, lipids, C-reactive protein, interleukin-6, and fibrinogen: a phase Ilb randomized, placebo-controlled trial. Circulation 2012; 126:2739-48.
16. Siemes C, Visser L E, Coebergh J W, Splinter T A, Witteman J C, Uitterlinden A G, Hofman A, Pols H A, Stricker B H. C-reactive protein levels, variation in the C-reactive protein gene, and cancer risk: the Rotterdam Study. J Clin Oncol 2006; 24:5216-22.
17. Allin K H, Bojesen S E, Nordestgaard B G. Baseline C-reactive protein is associated with incident cancer and survival in patients with cancer. J Clin Oncol 2009; 27:2217-24.
18. Chaturvedi A K, Caporaso N E, Katki H A, Wong H L, Chatterjee N, Pine S R, Chanock S J, Goedert J J, Engels E A. C-reactive protein and risk of lung cancer. J Clin Oncol 2010; 28:2719-26.
19. Ridker P M, Howard C P, Walter V, Everett B, Libby P, Hensen J, et al. Effects of interleukin-113 inhibition with canakinumab on hemoglobin A1c, lipids, C-reactive protein, interleukin-6, and fibrinogen: a phase IIb randomized, placebo-controlled trial. Circulation. 2012; 126:2739-48.
20. Ridker et al, CANTOS CVD manuscript
21. Carmi Y, Rinott G, Dotan S, Elkabets M, Rider P, Voronov E, Apte R N. Microenvironmental-derived IL-1 and IL-17 interact in the control of lung metastasis. J Immunol 2011; 186:3462-3471.
22. Balkwill F, Mantovani A. Inflammation and cancer: back to Virchow? Lancet 2001; 357:539-45.
23. Cuzick J, Otto F, Baron J A, Brown P H, Burn J, Greenwald P, Jankowski J, La Vecchia C, Meyskens F, Senn H J, Thun M. Aspirin and non-steroidal anti-inflammatory drugs for cancer prevention: an international consensus statement. Lancet Oncol 2009; 10:501-7.
24. Rothwell P M, Fowkes F G, Belch J F, Ogawa H, Warlow C P, Meade T W. Effect of daily aspirin on long-term risk of death due to cancer: analysis of individual patient data from randomised trials. Lancet 2011; 377:31-41.
25. Lust J A, Lacy M Q Zeldenrust S R, Dispenzieri A, Gertz M A, Witzig T E, Kumar S, Hayman S R, Russell S J, Buadi F K, Geyer S M, Campbell M E, Kyle R A, Rajkumar S V, Greipp P R, Kline M P, Xiong Y, Moon-Tasson L L, Donovan K A. Induction of a chronic disease state in patients with smoldering or indolent multiple myeloma by targeting interleukin 1{β}-induced interleukin 6 production and the myeloma proliferative component. Mayo Clin Proc 2009; 84:114-22.
26. Hong D S, Hui D, Bruera E, Janku F, Naing A, Falchook G S, Piha-Paul S, Wheler J J, Fu S, Tsimberidou A M, Stecher M, Mohanty P, Simard J, Kurzrock R. MABp1, a first-in-class true human antibody targeting interleukin-1alpha in refractory cancers: an open-label, phase 1 dose-escalation and expansion study. Lancet Oncol 2014; 15:656-66.

1-37. (canceled)

38. A method of treating a cancer having at least a partial inflammatory basis, comprising administering a therapeutically effective amount of canakinumab to a patient in need, wherein the patient has a high sensitivity C-reactive protein (hsCRP) level equal to or greater than about 2 mg/L before first administration of canakinumab, and wherein the cancer is a lung cancer.

39. The method of claim 38, wherein the lung cancer is a non-small cell lung cancer (NSCLC).

40. The method of claim 38, wherein the patient has a high sensitivity C-reactive protein (hsCRP) level equal to or greater than 10 mg/L before first administration of canakinumab.

41. The method of claim 38, wherein the patient has a high sensitivity C-reactive protein (hsCRP) level equal to or greater than 6 mg/L before first administration of canakinumab.

42. The method of claim 38, wherein the high sensitivity C-reactive protein (hsCRP) level of the patient has reduced to below about 2.3 mg/L when assessed at least about 3 months after the first administration of canakinumab.

43. The method of claim 38, wherein the high sensitivity C-reactive protein (hsCRP) level of the patient has reduced to below about 2 mg/L when assessed at least about 3 months after the first administration of canakinumab.

44. The method of claim 38, wherein the high sensitivity C-reactive protein (hsCRP) level of the patient has reduced to below about 1.8 mg/L when assessed at least about 3 months after the first administration of canakinumab.

45. The method of claim 38, wherein the high sensitivity C-reactive protein (hsCRP) level of the patient has reduced by at least 20% compared to baseline when assessed at least about 3 months after the first administration of canakinumab.

46. The method of claim 38, wherein the patient has a baseline level of interleukin-6 (IL-6) prior to the first administration of canakinumab, and wherein the IL-6 level of the patient has reduced by at least 20% compared to baseline when assessed at least about 3 months after the first administration of canakinumab.

47. The method of claim 38, wherein the therapeutically effective amount of canakinumab is about 90 mg to about 450 mg.

48. The method of claim 38, wherein the therapeutically effective amount of canakinumab is about 200 mg to about 450 mg.

49. The method of claim 38, wherein canakinumab is administered every two, three, four, or six weeks.

50. The method of claim 38, wherein canakinumab is administered in combination with one or more therapeutic agents.

51. The method of claim 50, wherein the therapeutic agent is a platinum based chemotherapy or a platinum-based doublet chemotherapy (PT-DC), a tyrosine kinase inhibitor, or a checkpoint inhibitor.

59. The method of claim 50, wherein the therapeutic agent is a PD-1 or PD-L1 inhibitor selected from the group consisting of nivolumab, pembrolizumab, atezolizumab, durvalumab, avelumab, and spartalizumab (PDR-001).

60. A method of treating a cancer having at least a partial inflammatory basis, comprising administering about 90 mg to about 450 mg of canakinumab to a patient in need, wherein the cancer is a lung cancer.

61. The method of claim 60, wherein the lung cancer is a non-small cell lung cancer (NSCLC).

62. The method of claim 60, wherein about 200 mg to about 400 mg of canakinumab is administered to the patient.

63. The method of claim 60, wherein canakinumab is administered in combination with one or more therapeutic agents.

64. The method of claim 63, wherein the therapeutic agent is a PD-1 or PD-L1 inhibitor selected from the group consisting of nivolumab, pembrolizumab, atezolizumab, durvalumab, avelumab, and spartalizumab (PDR-001).


SEQUENCE LISTING (1010 sequences)

SEQ ID NO: 1-505 - skipped (505 entries)

SEQ ID NO: 506 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507-515 - skipped (9 entries)

SEQ ID NO: 516 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517-519 - skipped (3 entries)

SEQ ID NO: 520 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521-605 - skipped (85 entries)

SEQ ID NO: 606 - protein, 120 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607-615 - skipped (9 entries)

SEQ ID NO: 616 - protein, 107 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617-619 - skipped (3 entries)

SEQ ID NO: 620 - protein, 120 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621-623 - skipped (3 entries)

SEQ ID NO: 624 - protein, 107 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625-705 - skipped (81 entries)

SEQ ID NO: 706 - protein, 125 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707-717 - skipped (11 entries)

SEQ ID NO: 718 - protein, 107 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719-723 - skipped (5 entries)

SEQ ID NO: 724 - protein, 125 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725-729 - skipped (5 entries)

SEQ ID NO: 730 - protein, 107 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731-761 - skipped (31 entries)

SEQ ID NO: 762 - protein, 447 aa, Artificial Sequence
  source: /note=“Description of Artificial Sequence: Synthetic polypeptide”
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, Artificial Sequence
  source: /note=“Description of Artificial Sequence: Synthetic polypeptide”
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, Artificial Sequence
  source: /note=“Description of Artificial Sequence: Synthetic polypeptide”
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, Artificial Sequence
  source: /note=“Description of Artificial Sequence: Synthetic polypeptide”
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766-805 - skipped (40 entries)

SEQ ID NO: 806 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807-815 - skipped (9 entries)

SEQ ID NO: 816 - protein, 111 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817-821 - skipped (5 entries)

SEQ ID NO: 822 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823-825 - skipped (3 entries)

SEQ ID NO: 826 - protein, 111 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827-829 - skipped (3 entries)

SEQ ID NO: 830 - protein, 114 aa, Artificial Sequence
  source: /note=“Description of Artificial Sequence: Synthetic polypeptide”
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  source: /note=“Description of Artificial Sequence: Synthetic polypeptide”
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  source: /note=“Description of Artificial Sequence: Synthetic polypeptide”
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  source: /note=“Description of Artificial Sequence: Synthetic polypeptide”
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903-919 - skipped (17 entries)

SEQ ID NO: 920 - protein, 124 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, Artificial Sequence
  source: /note=“Description of Artificial Sequence: Synthetic polypeptide”
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922-1000 - skipped (79 entries)

SEQ ID NO: 1001 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1002 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 1003 - protein, 114 aa, Artificial Sequence
  source: /note=“Description of Artificial Sequence: Synthetic polypeptide”
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1004 - protein, 297 aa, Artificial Sequence
  source: /note=“Description of Artificial Sequence: Synthetic polypeptide”
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1005 - protein, 114 aa, Artificial Sequence; identical to SEQ ID NO: 1001
  source: /note=“Description of Artificial Sequence: Synthetic polypeptide”
  VARIANT (93)..(93): /replace=“Lys”
  MISC_FEATURE (1)..(114): /note=“Variant residues given in the sequence have no preference with respect to those in the annotations for variant positions”

SEQ ID NO: 1006 - protein, 77 aa, Artificial Sequence
  source: /note=“Description of Artificial Sequence: Synthetic polypeptide”
ITCPPPMSVEHADIWVKSYSLYSRERYICNSGFKRKAGTSSLTECVLNKATNVAHWTTPSLKCIRDPALVHQRPAPP

SEQ ID NO: 1007 - protein, 214 aa, Artificial Sequence
  source: /note=“Description of Artificial Sequence: Synthetic polypeptide”
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1008 - protein, 448 aa, Artificial Sequence
  source: /note=“Description of Artificial Sequence: Synthetic polypeptide”
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1009 - protein, 445 aa, Artificial Sequence
  source: /note=“Description of Artificial Sequence: Synthetic polypeptide”
QVQLQESGPGLVKPSQTLSLTCSFSGFSLSTSGMGVGWIRQPSGKGLEWLAHIWWDGDESYNPSLKSRLTISKDTSKNQVSLKITSVTAADTAVYFCARNRYDPPWFVDWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 1010 - protein, 214 aa, Artificial Sequence
  source: /note=“Description of Artificial Sequence: Synthetic polypeptide”
DIQMTQSTSSLSASVGDRVTITCRASQDISNYLSWYQQKPGKAVKLLIYYTSKLHSGVPSRFSGSGSGTDYTLTISSLQQEDFATYFCLQGKMLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC